(19) United States
(12) Patent Application Publication
(10) Pub. No.: US 2024/0218353 A1
(43) Pub. Date: Jul. 4, 2024

(54) ALTERNATIVE RNA PURIFICATION STRATEGIES

(71) Applicant: ModernaTX, Inc., Cambridge, MA (US)

(72) Inventors: Christopher Ladd Effio, Cambridge, MA (US); Tahir Kapoor, Cambridge, MA (US); Joseph Elich, Cambridge, MA (US); Amy E. Rabideau, Waltham, MA (US)

(73) Assignee: ModernaTX, Inc., Cambridge, MA (US)

(21) Appl. No.: 18/570,927

(22) PCT Filed: Jun. 16, 2022
(86) PCT No.: PCT/US2022/033884

§ 371 Date: Dec. 15, 2023

Related U.S. Application Data
(60) Provisional application No. 63/316,672, filed on Mar. 4, 2022.
(60) Provisional application No. 63/286,249, filed on Dec. 6, 2021.
(60) Provisional application No. 63/212,057, filed on Jun. 17, 2021.
(60) Provisional application No. 63/270,737, filed on Oct. 22, 2021.

(51) Int. Cl. C12N 15/10; B01D 15/32; C12N 9/64

(52) U.S. Cl. CPC C12N 15/101; B01D 15/327; C12N 9/6424; C12Y 304/21014

(57) ABSTRACT

Provided herein are methods of purifying nucleic acids (e.g., mRNAs) from mixtures, including protease digestion of residual proteins, RNase III digestion of double-stranded RNAs, salt precipitation of mRNA, continuous removal of transcribed RNA, and improvement of column chromatography using high salt concentrations.

### RELATED APPLICATIONS

This application claims the benefit under 35 U.S.C. § 119(e) of U.S. provisional Application No. 63/212,057 filed Jun. 17, 2021, U.S. provisional Application No. 63/270,737 filed Oct. 22, 2021, U.S. provisional Application No. 63/286,249 filed Dec. 6, 2021, and U.S. provisional Application No. 63/316,672 filed Mar. 4, 2022, each of which is incorporated by reference herein in its entirety.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Jun. 14, 2022, is named M137870189W000-SEQ-NTJ, and is 25,661 bytes in size.

### BACKGROUND

Messenger RNA (mRNA) is an emerging alternative to conventional small molecule and protein therapeutics due to the potency and programmability of mRNA. mRNA encoding a desired therapeutic protein can be administered to a subject for in vivo expression of the protein to therapeutic effect, such as vaccination or replacement of a protein encoded by a mutated gene. In vitro transcription of a DNA template using a bacteriophage RNA polymerase is a useful method of producing mRNAs for therapeutic applications, but in vitro transcribed mRNAs must be purified before downstream use.

### SUMMARY

Provided herein are methods (e.g., column-free methods) of purifying nucleic acids, such as mRNA, from an in vitro transcription reaction. In vitro transcription, in which an RNA polymerase uses a DNA template to produce an RNA transcript, is useful for generating mRNA, but the presence of IVT reaction components, including DNA templates, DNases used to cleave DNA templates, and RNA polymerases, can inhibit the ability of mRNA to be translated and/or catalyze degradation of the mRNA. mRNAs may be purified from IVT reactions by column chromatography, but alternative methods that make more efficient use of columns or chromatography reagents, or eliminate the need for columns, can reduce the cost and improve the efficiency of the purification process. In one example, DNases are used to degrade DNA templates after IVT, while proteases are used to degrade the DNases and RNA polymerases into smaller peptide fragments, with peptide fragments and proteases being separated from the mRNA by tangential flow filtration. In another example, RNase III is used to digest dsRNAs, such as double-stranded regions of mRNA transcripts, into smaller dsRNA fragments that can be separated from full-length mRNA transcripts based on their smaller size, such as through tangential flow filtration. In another example, DNases are used to degrade DNA templates, and salt precipitation is used to precipitate mRNA, so that dissolved proteins and other components can be removed by washing before the mRNA is resuspended in a pure solution. Additionally, because mRNA can inhibit in vitro transcription at sufficient concentrations, methods may include the step of harvesting transcribed mRNA from an IVT reaction, such as by oligo-dT filtration. Removal of produced mRNA, while returning DNA templates, RNA polymerases, and nucleotide triphosphates to the IVT reaction, avoids the self-limiting effects of mRNA in in vitro transcription. Finally, salt may be added to an mRNA composition shortly before column chromatography to increase the amount of mRNA that can be bound by a given column, for instance by making mRNA molecules more compact. Increasing column binding capacity in this manner allows more mRNA to be purified from a given column in the same amount of time, thereby increasing the productivity of column chromatography. Also provided are compositions produced by the methods provided herein, and compositions comprising mRNAs formulated in lipid nanoparticles with minimal protein concentrations.

Accordingly, some aspects of the disclosure relate to a method of purifying in vitro transcribed mRNA, the method comprising:
(a) adding a high-salt buffer to a composition comprising mRNA to produce a high-salt mRNA composition comprising a salt concentration of at least 100 mM;
(b) contacting a stationary phase with the composition produced in (a); and
(c) eluting mRNA from the stationary phase of (b) to obtain eluted mRNA.

In some embodiments, the stationary phase of (b) comprises fiber, particles, resin, beads, a membrane, and/or monolithic stationary phase.

In some embodiments, the stationary phase of (b) comprises an oligonucleotide comprising a nucleic acid sequence that is complementary to a nucleotide sequence of the mRNA.

In some embodiments, the stationary phase of (b) comprises oligo-dT resin.

In some embodiments, the stationary phase of (b) comprises a hydrophobic interaction chromatography (HIC) ligand, optionally wherein the HIC ligand comprises a butyl, phenyl, octyl, t-butyl, methyl, and/or ethyl functional group.

In some embodiments, the high-salt mRNA composition has a salt concentration of at least 200 mM, at least 300 mM, at least 400 mM, at least 500 mM, at least 600 mM, at least 700 mM, at least 800 mM, at least 900 mM, at least 1 M, or more.

In some embodiments, the high-salt mRNA composition has a salt concentration of about 400 mM to about 600 mM, optionally wherein the high-salt mRNA composition has a salt concentration of about 500 mM.

In some embodiments, the salt concentration of the high-salt mRNA composition is the concentration of sodium chloride, potassium chloride, ammonium chloride, ammonium sulfate, monosodium phosphate, disodium phosphate, or trisodium phosphate in the composition.

In some embodiments, the high-salt mRNA composition comprises a sodium chloride concentration of about 400 mM to about 600 mM, optionally wherein the composition has a sodium chloride concentration of about 500 mM.

In some embodiments, the contacting of (b) occurs within 1 hour or less, 30 minutes or less, 25 minutes or less, 20 minutes or less, 15 minutes or less, 10 minutes or less, 5 minutes or less, 4 minutes or less, 3 minutes or less, 2 minutes or less, or 1 minute or less, of the adding of the high-salt buffer to the composition comprising mRNA of (a).

In some embodiments, the high-salt buffer is added by in-line mixing.

In some embodiments, the high-salt buffer is added by bolus addition.

In some embodiments, the method further comprises desalting the composition comprising mRNA before adding the high-salt buffer of (a) to produce a desalted mRNA composition with a salt concentration of less than 20 mM.

In some embodiments, the desalting comprises binding the mRNA composition to a hydrophobic interaction chromatography (HIC) resin and eluting the mRNA from the HIC resin to produce a desalted mRNA composition.

In some embodiments, the high-salt mRNA composition comprises at least 2.0 g/L, 2.5 g/L, 3.0 g/L, 3.5 g/L, 4.0 g/L, 4.5 g/L, 5.0 g/L, 6.0 g/L, 7.0 g/L, 8.0 g/L, 9.0 g/L, 10.0 g/L, or more dissolved mRNA.

In some embodiments, the high-salt mRNA composition of (a) comprises about 4.0 g/L to about 6.0 g/L dissolved mRNA.

In some embodiments, at least 80%, at least 85%, at least 90%, at least 95%, at least 97%, at least 98%, at least 99%, or up to 100% of mRNAs in the high-salt mRNA composition are dissolved mRNAs.

In some embodiments, the stationary phase of (b) is comprised in a column, wherein the concentration of mRNA in the composition of (b) is 100% or less, 90% or less, or 80% or less of the dynamic binding capacity of the column.

In some embodiments, the mRNA is produced by an in vitro transcription step comprising:
in a reaction vessel comprising a mixture comprising a DNA molecule, nucleotide triphosphates (NTPs) including adenosine triphosphate (ATP), cytidine triphosphate (CTP), uridine triphosphate (UTP), and guanosine triphosphate (GTP), and an RNA polymerase, in vitro transcribing a DNA molecule, whereby the RNA polymerase transcribes the DNA molecule to produce an in vitro transcribed mRNA.

In some embodiments, the method further comprises:
(i) contacting the mixture with a protease;
(ii) incubating the mixture for a period of time sufficient for the protease to cleave one or more proteins in the mixture to produce peptide fragments; and
(iii) isolating the mRNA from the mixture to obtain an isolated mRNA composition.

Some aspects relate to a method of purifying in vitro transcribed mRNA, the method comprising:
(i) contacting a solution comprising the mRNA with a protease;
(ii) incubating the mixture for a period of time sufficient for the protease to cleave one or more proteins in the mixture to produce peptide fragments, and
(iii) isolating the mRNA from the mixture to obtain an isolated mRNA composition.

In some embodiments, wherein the protease is selected from the group consisting of proteinase K, Lys-C, trypsin, TPCK-treated trypsin, chymotrypsin, α-lytic protease, and endoproteinase AspN.

In some embodiments, the protease is proteinase K.

In some embodiments, the proteinase K comprises an amino acid sequence with at least 90%, at least 91%, at least 92%, at least 93%, at least 94%, at least 95%, at least 96%, at least 97%, at least 98%, at least 99%, or up to 100% sequence identity to the amino acid sequence of SEQ ID NO: 3.

In some embodiments, the proteinase K comprises the amino acid sequence of SEQ ID NO: 3.

In some embodiments, the protease is at a concentration of about 0.1 to about 100 Units/mL, about 0.2 to about 50 Units/mL, about 0.3 to about 25 Units/mL, about 0.4 to about 10 Units/mL, about 0.5 to about 5 Units/mL, about 0.5 to about 3 Units/mL, about 0.5 to about 2 Units/mL, or about 0.5 to about 1 Unit/mL.

In some embodiments, the concentration of the protease is about 0.1 to about 2 Units/mL.

In some embodiments, the protease:protein concentration in the mixture is about 1:10 to about 1:100, about 1:100 to about 1:1,000, about 1:1,000 to about 1:10,000, about 1:10,000 to about 1:100,000, or about 1:100,000 to about 1:1,000,000.

In some embodiments, the protease:protein concentration in the mixture is about 1:1,000 to about 1:50,000.

In some embodiments, the mixture of (i) comprises one or more cations.

In some embodiments, step (i) and/or step (ii) comprises adding one or more cations to the mixture.

In some embodiments, the cation is a magnesium ion or a calcium ion.

In some embodiments, the concentration of magnesium ions in the mixture during step (ii) is about 10 mM to about 100 mM.

In some embodiments, the incubating of step (ii) is conducted at about 37° C.

In some embodiments, wherein the incubating of step (ii) is conducted for about 10 minutes to about 6 hours.

In some embodiments, the isolating step (iii) comprises separating the mRNA from the protease and peptide fragments by tangential flow filtration (TFF).

In some embodiments, the tangential flow filtration comprises using a TFF membrane with a molecular weight cutoff of 500 kDa or less, 200 kDa or less, or 100 kDa or less.

In some embodiments, the tangential flow filtration comprises using a TFF membrane with a molecular weight cutoff of 100 kDa or less.

In some embodiments, the contacting of step (i) further comprises contacting the mixture comprising the mRNA with a DNase, and wherein the incubating of step (ii) further comprises incubating the mixture for a period of time sufficient for the DNase to cleave one or more DNAs in the mixture to produce DNA fragments.

In some embodiments, the in vitro transcribed mRNA is produced by a method comprising the steps of:
(i) in a reaction vessel comprising a mixture comprising a DNA molecule and an RNA polymerase, in vitro transcribing a DNA molecule, whereby the RNA polymerase transcribes the DNA molecule to produce an mRNA, wherein mRNA is removed from the reaction vessel by the steps of:
   (1) transferring a portion of the mixture from the reaction vessel to a column comprising a stationary phase;
   (2) passing the portion of the mixture through the column, whereby the stationary phase retains mRNA from the mixture; and
   (3) re-introducing a flowthrough from the column into the reaction vessel, wherein the concentration of mRNA in the flowthrough of step (3) is lower than the concentration of mRNA in the portion of the mixture of step (1).

In some aspects, the disclosure relates to a method of removing in vitro transcribed mRNA from an in vitro transcription reaction, the method comprising:
(i) in a reaction vessel comprising a mixture comprising a DNA molecule and an RNA polymerase, in vitro transcribing a DNA molecule, whereby the RNA polymerase transcribes the DNA molecule to produce an RNA, wherein RNA is removed from the reaction vessel by the steps of:
   (1) transferring a portion of the mixture from the reaction vessel to a column comprising a stationary phase;
   (2) passing the portion of the mixture through the column, whereby the stationary phase retains RNA from the mixture; and
   (3) re-introducing a flowthrough from the column into the reaction vessel, wherein the concentration of RNA in the flowthrough of step (3) is lower than the concentration of RNA in the portion of the mixture of step (1); and
(ii) isolating the mRNA from the mixture to obtain an isolated mRNA composition.

In some embodiments, the method further comprises, prior to the isolation of step (ii), contacting the mixture with a DNase, and incubating the mixture for a period of time sufficient for the DNase to cleave one or more DNAs in the mixture to produce DNA fragments.

In some embodiments, the stationary phase comprises fiber, particles, resin, and/or beads.

In some embodiments, the stationary phase comprises oligo-dT.

In some embodiments, the stationary phase comprises oligo-dT fiber.

In some embodiments, the concentration of NTPs in the reaction vessel is between about 30 mM and about 50 mM.

In some embodiments:
(a) the concentration of GTP in the reaction mixture is at least 2× the concentration of each of ATP, CTP, and UTP;
(b) the reaction mixture further comprises guanosine diphosphate (GDP), and wherein the concentration of GDP is at least 2× the concentration of each of ATP, CTP, and UTP; and/or
(c) the reaction mixture further comprises GDP, and wherein the ratio of concentration of GTP plus GDP to the concentration of each of ATP, CTP, and UTP is at least 2:1.

In some embodiments, the ratio of concentrations of GTP:ATP:CTP:UTP is 4:2:1:1, 4:2:2:1, or 6:3:3:1.

In some embodiments, the in vitro transcribing step of (i) further comprises adding a feed solution comprising GTP, ATP, CTP, and UTP.

In some embodiments:
(a) 25-35% of NTPs in the feed solution are GTP;
(b) 20-30% of NTPs in the feed solution are ATP;
(c) 30-40% of NTPs in the feed solution are CTP; and/or
(d) 10-20% of NTPs in the feed solution are UTP.

In some embodiments, after addition of the feed solution, the concentration of GTP in the reaction mixture is at least 2× the concentration of each of ATP, CTP, and UTP.

In some embodiments, the feed solution further comprises GDP, wherein, after addition of the feed solution:
(a) the concentration of GDP is at least 2× the concentration of each of ATP, CTP, and UTP; and/or
(b) the ratio of concentration of GTP plus GDP to the concentration of each of ATP, CTP, and UTP is at least 2:1.

In some embodiments, after addition of the feed solution:
(a) the ratio of GTP:ATP is in the reaction mixture is 1.5:1 to 2.5:1, the ratio of GTP:CTP is in the reaction mixture is 3.5:1 to 4.5:1, and the ratio of GTP:UTP is in the reaction mixture is 3.5:1 to 4.5:1;
(b) the ratio of GTP:ATP is in the reaction mixture is 1.5:1 to 2.5:1, the ratio of GTP:CTP is in the reaction mixture is 1.5:1 to 2.5:1, and the ratio of GTP:UTP is in the reaction mixture is 3.5:1 to 4.5:1; or
(c) the ratio of GTP:ATP is in the reaction mixture is 1.5:1 to 2.5:1, the ratio of GTP:CTP is in the reaction mixture is 1.5:1 to 2.5:1, and the ratio of GTP:UTP is in the reaction mixture is 5.5:1 to 6.5:1 In some embodiments, after addition of the feed solution, the ratio of concentrations of GTP:ATP:CTP:UTP in the reaction mixture is 4:2:1:1, 4:2:2:1, or 6:3:3:1.

In some embodiments, the feed solution further comprises magnesium ions.

In some embodiments, the concentration of magnesium ions in the reaction vessel, after addition of the feed solution, is between 200 mM and 500 mM.

In some embodiments, the feed solution is added to the reaction vessel continuously.

In some embodiments, the feed solution is added to the reaction vessel as a bolus.

In some embodiments, the method further comprises reducing the volume of the reaction mixture.

In some embodiments, reducing the volume of the reaction mixture comprises tangential flow filtration (TFF).

In some embodiments, the tangential flow filtration comprises using a TFF membrane with a molecular weight cutoff of 50 kDa or less.

In some embodiments, the concentration of mRNA in the reaction vessel is maintained at a concentration below 20 mg/mL, below 15 mg/mL, below 12 mg/mL, or below 10 mg/mL.

In some embodiments, the concentration of mRNA in the reaction vessel is maintained at a concentration of 8 mg/mL or more, 9 mg/mL or more, 10 mg/mL or more, or 11 mg/mL or more.

In some embodiments, the method further comprises eluting mRNA from the column to collect an eluate comprising mRNA.

In some embodiments, the eluting is performed more than once.

In some embodiments, the steps of (1), (2), and (3) are repeated after the eluting step.

In some embodiments, the steps of (1), (2), and (3) are performed continuously, paused before elution, restarted after elution, and performed continuously after elution.

In some embodiments, the eluate is added to the mixture prior to the isolating of step (ii).

In some embodiments, the method further comprises:
(a) contacting the mixture with an RNase III;
(b) incubating the RNase III to cleave one or more double-stranded RNAs (dsRNAs) in the mixture; and
(c) isolating mRNA from the mixture to obtain an isolated mRNA composition.

In some aspects, the disclosure relates to a method of reducing double-stranded RNA in an mRNA composition, the method comprising:
(i) in a reaction vessel comprising a mixture comprising a DNA molecule and an RNA polymerase, in vitro transcribing a DNA molecule, whereby the RNA polymerase transcribes the DNA molecule to produce an mRNA;
(ii) contacting the mixture with an RNase III;
(iii) incubating the RNase III to cleave one or more double-stranded RNAs (dsRNAs) in the mixture; and
(iv) isolating the mRNA from the mixture to obtain an isolated mRNA composition.

In some embodiments, the RNase III is present in the mixture during the in vitro transcribing step, wherein the step of incubating the RNase III is conducted during the in vitro transcribing step.

In some embodiments, the RNase III is added to the in vitro transcription mixture after 30 minutes, 60 minutes, 90 minutes, 100 minutes, 110 minutes, 120 minutes, 130 minutes, 140 minutes, 150 minutes, 160 minutes, or 170 minutes of in vitro transcription.

In some embodiments, the RNase III comprises an amino acid sequence with at least 80% sequence identity to the amino acid sequence of SEQ ID NO: 4, wherein the RNase III comprises an amino acid substitution corresponding to an E38A substitution in SEQ ID NO: 5.

In some embodiments, the RNase III comprises the amino acid sequence of SEQ ID NO: 5.

In some embodiments, the mixture comprises magnesium ions during the step of incubating the RNase III.

In some embodiments, the concentration of magnesium ions in the mixture during the step of incubating the RNase III is between about 10 mM to about 100 mM, optionally wherein the concentration of magnesium ions is about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, or about 30 mM.

In some embodiments, the concentration of RNase III in the mixture during the step of incubating the RNase III is less than 0.2 U/mL, less than 0.15 U/mL, less than 0.1 U/mL, less than 0.09 U/mL, less than 0.08 U/mL, less than 0.07 U/mL, less than 0.06 U/mL, or less than 0.05 U/mL.

In some embodiments, the RNase III is incubated for a period of time sufficient to cleave at least 80%, at least 90%, at least 95%, at least 97%, at least 98%, at least 99%, or up to 100% of dsRNAs in the mixture.

In some embodiments, the RNase III is incubated for about 1 minute to about 5 minutes, about 5 minutes to about 10 minutes, about 10 minutes to about 20 minutes, about 20 minutes to about 30 minutes, about 30 minutes to about 40 minutes, about 40 minutes to about 50 minutes, or about 50 minutes to about 60 minutes.

In some embodiments, the disclosure relates to an isolated mRNA composition comprising mRNA produced by any one of the methods provided herein.

In some embodiments, the concentration of proteins in the isolated mRNA composition of step (ii) is 0.8% (% w/w) or less, 0.6% or less, 0.4% or less, or 0.2% or less.

In some embodiments, at least 50%, at least 60%, at least 70%, at least 75%, at least 80%, at least 85%, or at least 90% of the mRNA molecules of the isolated mRNA composition comprise a poly(A) tail.

In some embodiments, the mRNA is formulated in a lipid nanoparticle.

In some embodiments, the lipid nanoparticle comprises: an ionizable amino lipid.

In some embodiments, the lipid nanoparticle further comprises: a non-cationic lipid; a sterol; and a polyethylene glycol (PEG)-modified lipid.

In some embodiments, the lipid nanoparticle comprises: 40-55 mol % ionizable amino lipid; 5-15 mol % non-cationic lipid; 35-45 mol % sterol; and 1-5 mol % PEG-modified lipid.

In some embodiments, the disclosure relates to a composition comprising mRNA formulated in a lipid nanoparticle, wherein a concentration of proteins in the mRNA prior to formulation in the lipid nanoparticle is 0.8% (% w/w) or less, 0.6% or less, 0.4% or less, or 0.2% or less, and wherein at least 50%, at least 60%, at least 70%, at least 75%, at least 80%, at least 85%, or at least 90% of the mRNA molecules of the mRNA composition comprise a poly(A) tail.

In some aspects, the disclosure relates to a composition comprising:
(i) an mRNA;
(ii) a DNA;
(iii) one or more nucleotide triphosphates;
(iv) one or more proteins or peptide fragments thereof;
(v) a protease in an amount sufficient to cleave one or more proteins in the mixture into peptide fragments.

In some embodiments, the composition comprises one or more proteins and one or more peptide fragments thereof.

In some embodiments, at least 80%, at least 85%, at least 90%, at least 95%, at least 96%, at least 97%, at least 98%, at least 99%, or up to 100% of the proteins or peptide fragments in the mixture are 100 kDa or less in size.

In some embodiments, the protease is selected from the group consisting of proteinase K, Lys-C, trypsin, TPCK-treated trypsin, chymotrypsin, α-lytic protease, and endoproteinase AspN.

In some embodiments, the protease is proteinase K.

In some embodiments, the proteinase K comprises an amino acid sequence with at least 90%, at least 91%, at least 92%, at least 93%, at least 94%, at least 95%, at least 96%, at least 97%, at least 98%, at least 99%, or up to 100% sequence identity to the amino acid sequence of SEQ ID NO: 3.

In some embodiments, the proteinase K comprises the amino acid sequence of SEQ ID NO: 3.

In some embodiments, the protease is at a concentration of about 0.1 to about 100 Units/mL, about 0.2 to about 50 Units/mL, about 0.3 to about 25 Units/mL, about 0.4 to about 10 Units/mL, about 0.5 to about 5 Units/mL, about 0.5 to about 3 Units/mL, about 0.5 to about 2 Units/mL, or about 0.5 to about 1 Unit/mL.

In some embodiments, the concentration of the protease is about 0.1 to about 2 Units/mL.

In some embodiments, the composition comprises one or more cations.

In some embodiments, the cation is a magnesium ion or a calcium ion.

In some embodiments, the concentration of magnesium ions in the mixture is about 10 mM to about 100 mM.

### BRIEF DESCRIPTION OF THE DRAWINGS

FIGS. 1A-1F show the effects of protease digestion in removing residual proteins from in vitro transcribed mRNA. FIG. 1A shows the % mRNA purity (lines) and % residual protein (bars) present in the mixture at the start of the reaction, after 60 minutes, or after 120 minutes of incubation with 2 μg/mL, 20 μg/mL, or 200 μg/mL protease. FIG. 1B shows an electropherogram of an RNA (2K construct) in the absence (R0) or presence (R0+PK) of proteinase K. Fluorescence at a given alignment time indicates the abundance of a given protein. FIG. 1C shows an HPLC chromatogram in which a single peak, corresponding to a linear mRNA, was observed after digestion of an IVT reaction with proteinase K. FIG. 1D shows % RNA purity, for three RNA constructs, of control-treated (left) and protease-treated (right) IVT mixtures. FIG. 1E shows the concentration of double-stranded RNA (dsRNA) (% w/w) of the same three constructs in FIG. 1D, before and after incubation with a protease. FIG. 1F shows the concentration of protein, as a percentage of the mass of mRNA, of the constructs shown in FIGS. 1D-1E, after incubation with a protease.

FIGS. 2A-2D show the effects of different proteases and reaction conditions on the effectiveness of protease digestion for removing residual proteins from an IVT reaction. FIG. 2A shows the abundance of proteins after digestion of an IVT reaction with one of multiple different proteases. FIG. 2B shows the abundance of proteins after incubation with proteinase K under various reaction conditions, for varying amounts of time. * indicates the presence of 20 mM Mg²⁺. FIG. 2C shows the abundance of proteins after incubation with proteinase K at varying concentrations. FIG. 2D shows the abundance of proteins after incubation with various concentrations of proteinase K, relative to the concentration of IVT enzymes.

FIGS. 3A-3C show the effects of continuously removing in vitro transcribed RNA from an IVT reaction as it is produced, to prevent the inhibitor effects of mRNA on further IVT. FIG. 3A shows a flowchart describing the process of removing mRNA from an IVT reaction by filtration through an oligo-dT column, then reintroducing other components of the reaction mixture, such as DNA, NTPs, and IVT enzymes, back into the IVT reaction vessel. FIG. 3B shows the absorbance of different fractions obtained from multiple elutions form a continuous IVT reaction. FIG. 3C shows the mRNA mass (mg), and % purity of mRNAs, based on desired length and poly(A) tail content, for successive elutions of mRNA from a continuous IVT reaction.

FIGS. 4A-4D show the effects of salt concentration on the efficiency of mRNA purification using dT chromatography-based purification. FIG. 4A shows how increasing the scale of an IVT reaction increases the amount of buffer required (squares), dT chromatography elution volume (triangles), and column internal diameter (ID, inverted triangles) required for mRNA purification. FIGS. 4B-4D shows how intensifying the dT chromatography process reduces the required column internal diameter (FIG. 4B), amount of buffer required (FIG. 4C), and volume required for elution of purified mRNA (FIG. 4D). Dotted lines indicate maximum column diameter (FIGS. 4A-4B) and/or maximum elution volume (FIGS. 4A and 4D) in process.

FIGS. 5A-5C show the relationship between salt concentration and parameters of dT chromatography. FIG. 5A shows the relationship between salt concentration and column static binding capacity (SBC), dynamic binding capacity (DBC), and the purity, in terms of mRNAs having poly(A) tails and expected lengths. FIGS. 5B-5C show the relationship between salt concentration and the solubility of mRNA following salt addition and incubation either overnight at 4° C. (FIG. 5B) or for 1 hour at 25° C. (FIG. 5C).

FIGS. 6A-6C show the effects of two RNase III variants on the purity and dsRNA content of mRNA preparations. FIG. 6A shows the kinetics of mRNA size purity (% mRNAs having an expected length) during separate digestion by one of two variants of RNase III. FIG. 6B shows the kinetics of mRNA tail purity (% mRNAs having polyA tail of expected length) during separate digestion by one of two variants of RNase III. FIG. 6C shows the kinetics of dsRNA content (% mRNAs that are double-stranded) during separate digestion by one of two variants of RNase III.

FIGS. 7A-7E show the effects of varying concentrations of two RNase III variants on the size purity and dsRNA content of mRNA preparations. FIG. 7A shows the size purity of mRNA preparations digested with varying concentrations of RNase III variant 2. FIGS. 7B-7C show the kinetics of size purity of mRNAs digested with varying concentrations of RNase III variant 1 (FIG. 7B) or RNase III variant 2 (FIG. 7C). FIGS. 7D-7E show the kinetics of dsRNA content in mRNA preparations digested with varying concentrations of RNase III variant 1 (FIG. 7D) or RNase III variant 2 (FIG. 7E).

FIGS. 8A-8B show the effects of RNase III digestion on mRNAs of varying lengths. FIG. 8A shows the kinetics of size purity during separate digestion of mRNAs with different lengths by RNase III variant 2. FIG. 8B shows the kinetics of dsRNA content during separate digestion of mRNAs with different lengths by RNase III variant 2.

FIG. 9 shows an alignment between RNase III amino acid sequences of Escherichia coli, Thermotoga maritima, and Aquifex aeolicus.

### DETAILED DESCRIPTION

The disclosure relates to methods of purifying nucleic acids, such as mRNA, from an in vitro transcription (IVT) reaction. mRNA can be produced by IVT, but the presence of IVT reaction components, including DNA templates, DNases used to cleave DNA templates, and RNA polymerases, can inhibit the ability of mRNA to be translated and/or catalyze degradation of the mRNA. Thus, mRNAs must be separated from IVT reaction components before use in downstream applications, such as encapsulation in lipid nanoparticles and/or administration to subjects. mRNAs may be purified from IVT reactions by column chromatography, but alternative methods that make more efficient use of columns or chromatography reagents, or eliminate the need for columns, can reduce the cost and improve the efficiency of the purification process. For example, DNases can degrade DNA templates after IVT, while proteases degrade the DNases and RNA polymerases into smaller peptide fragments, with peptide fragments and proteases being separated from the mRNA by tangential flow filtration. In another example, RNase III is used to digest dsRNAs, such as double-stranded regions of mRNA transcripts, into smaller dsRNA fragments that can be separated from full-length mRNA transcripts based on their smaller size, such as through tangential flow filtration. In another example, DNases degrade DNA templates, and salts such as lithium chloride are used to precipitate mRNA, so that dissolved proteins and other components can be removed by washing before the mRNA is resuspended in a protein-free resuspension solution. Additionally, because mRNA can inhibit in vitro transcription at sufficient concentrations, methods may include the step of harvesting transcribed mRNA from an IVT reaction, such as by oligo-dT filtration. Removal of produced mRNA, while returning DNA templates, RNA polymerases, and nucleotide triphosphates to the IVT reaction, avoids the self-limiting effects of mRNA in in vitro transcription. Finally, salt may be added to an mRNA composition shortly before column chromatography to increase the amount of mRNA that can be bound by a given column, for instance by making mRNA molecules more compact. Increasing column binding capacity in this manner allows more mRNA to be purified from a given column in the same amount of time, thereby increasing the productivity of column chromatography. Also provided are compositions produced by the methods provided herein, and compositions comprising mRNAs formulated in lipid nanoparticles with minimal protein concentrations.

### Nucleic Acids

Aspects of the disclosure relate to compositions comprising nucleic acids and methods of producing nucleic acids. As used herein, the term “nucleic acid” includes multiple nucleotides (i.e., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G))). The term nucleic acid includes polyribonucleotides as well as polydeoxyribonucleotides. The term nucleic acid also includes polynucleosides (i.e., a polynucleotide minus the phosphate) and any other organic base containing polymer. Non-limiting examples of nucleic acids include chromosomes, genomic loci, genes or gene segments that encode polynucleotides or polypeptides, coding sequences, non-coding sequences (e.g., intron, 5′-UTR, or 3′-UTR) of a gene, pri-mRNA, pre-mRNA, cDNA, mRNA, etc. A nucleic acid (e.g., mRNA) may include a substitution and/or modification. In some embodiments, the substitution and/or modification is in one or more bases and/or sugars. For example, in some embodiments a nucleic acid (e.g., mRNA) includes nucleotides having an organic group, such as a methyl group, attached to a nucleic acid base at the N6 position. Thus, in some embodiments, an mRNA includes one or more N6-methyladenosine nucleotides. A phosphate, sugar, or nucleic acid base of a nucleotide may also be substituted for another phosphate, sugar, or nucleic acid base. For example, a uridine base may be substituted for a pseudouridine base, in which the uracil base is attached to the sugar by a carbon-carbon bond rather than a nitrogen-carbon bond. Thus, in some embodiments, a nucleic acid (e.g., mRNA) is heterogeneous in backbone composition thereby containing any possible combination of polymer units linked together such as peptide-nucleic acids (which have an amino acid backbone with nucleic acid bases).

The nucleic acid sequences of nucleic acids described herein include nucleic acid sequences that have been removed from their naturally occurring environment, recombinant or cloned DNA isolates, and chemically synthesized analogues or analogues biologically synthesized by heterologous systems.

An “engineered nucleic acid” is a nucleic acid that does not occur in nature. It should be understood, however, that while an engineered nucleic acid as a whole is not naturally-occurring, it may include nucleotide sequences that occur in nature. In some embodiments, an engineered nucleic acid comprises nucleotide sequences from different organisms (e.g., from different species). For example, in some embodiments, an engineered nucleic acid includes a bacterial nucleotide sequence, a human nucleotide sequence, and/or a viral nucleotide sequence.

Engineered nucleic acids include recombinant nucleic acids and synthetic nucleic acids. A “recombinant nucleic acid” is a molecule that is constructed by joining nucleic acids (e.g., isolated nucleic acids, synthetic nucleic acids or a combination thereof) and, in some embodiments, can replicate in a living cell. A “synthetic nucleic acid” is a molecule that is amplified or chemically, or by other means, synthesized. A synthetic nucleic acid includes those that are chemically modified, or otherwise modified, but can base pair with naturally-occurring nucleic acid molecules. Recombinant and synthetic nucleic acids also include those molecules that result from the replication of either of the foregoing. A nucleic may comprise naturally occurring nucleotides and/or non-naturally occurring nucleotides such as modified nucleotides.

In some embodiments, a nucleic acid is present in (or on) a vector. Examples of vectors include but are not limited to bacterial plasmids, phage, cosmids, phasmids, fosmids, bacterial artificial chromosomes, yeast artificial chromosomes, viruses and retroviruses (for example vaccinia, adenovirus, adeno-associated virus, lentivirus, herpes-simplex virus, Epstein-Barr virus, fowlpox virus, pseudorabies, baculovirus) and vectors derived therefrom. In some embodiments, a nucleic acid (e.g., DNA) used as an input molecule for in vitro transcription (IVT) is present in a plasmid vector.

When applied to a nucleic acid sequence, the term “isolated” denotes that the polynucleotide sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment.

In some embodiments, an input DNA for IVT is a nucleic acid vector. A “nucleic acid vector” is a polynucleotide that carries at least one foreign or heterologous nucleic acid fragment. A nucleic acid vector may function like a “molecular carrier”, delivering fragments of nucleic acids or polynucleotides into a host cell or as a template for IVT. An “in vitro transcription template” (IVT template), or “input DNA” as used herein, refers to deoxyribonucleic acid (DNA) suitable for use in an IVT reaction for the production of messenger RNA (mRNA). In some embodiments, an IVT template encodes a 5′ untranslated region, contains an open reading frame, and encodes a 3′ untranslated region and a polyA tail. The particular nucleotide sequence composition and length of an IVT template will depend on the mRNA of interest encoded by the template.

In some embodiments the nucleic acid vector is a circular nucleic acid such as a plasmid. In other embodiments it is a linearized nucleic acid. According to some embodiments the nucleic acid vector comprises a predefined restriction site, which can be used for linearization. The linearization restriction site determines where the vector nucleic acid is opened/linearized. The restriction enzymes chosen for linearization should preferably not cut within the critical components of the vector.

A nucleic acid vector may include an insert which may be an expression cassette or open reading frame (ORF). An “open reading frame” is a continuous stretch of DNA beginning with a start codon (e.g., methionine (ATG)), and ending with a stop codon (e.g., TAA, TAG or TGA) and encodes a protein or peptide (e.g., a therapeutic protein or therapeutic peptide). In some embodiments, an expression cassette encodes an RNA including at least the following elements: a 5′ untranslated region, an open reading frame region encoding the mRNA, a 3′ untranslated region and a polyA tail. The open reading frame may encode any mRNA sequence, or portion thereof.

In some embodiments, a nucleic acid vector comprises a 5′ untranslated region (UTR). A “5′ untranslated region (UTR)” refers to a region of an mRNA that is directly upstream (i.e., 5′) from the start codon (i.e., the first codon of an mRNA transcript translated by a ribosome) that does not encode a protein or peptide. 5′ UTRs are further described herein, for example in the section entitled “Untranslated Regions”.

In some embodiments, a nucleic acid vector comprises a 3′ untranslated region (UTR). A “3′ untranslated region (UTR)” refers to a region of an mRNA that is directly downstream (i.e., 3′) from the stop codon (i.e., the codon of an mRNA transcript that signals a termination of translation) that does not encode a protein or peptide. 3′ UTRs are further described herein, for example in the section entitled “Untranslated Regions”.

The terms 5′ and 3′ are used herein to describe features of a nucleic acid sequence related to either the position of genetic elements and/or the direction of events (5′ to 3′), such as e.g. transcription by RNA polymerase or translation by the ribosome which proceeds in 5′ to 3′ direction. Synonyms are upstream (5′) and downstream (3′). Conventionally, DNA sequences, gene maps, vector cards and RNA sequences are drawn with 5′ to 3′ from left to right or the 5′ to 3′ direction is indicated with arrows, wherein the arrowhead points in the 3′ direction. Accordingly, 5′ (upstream) indicates genetic elements positioned towards the left-hand side, and 3′ (downstream) indicates genetic elements positioned towards the right-hand side, when following this convention.

Aspects of the disclosure relate to populations of molecules. As used herein, a “population” of molecules (e.g., DNA molecules) generally refers to a preparation (e.g., a plasmid preparation) comprising a plurality of copies of the molecule (e.g., DNA) of interest, for example a cell extract preparation comprising a plurality of expression vectors encoding a molecule of interest (e.g., a DNA encoding an RNA of interest).

A nucleic acid (e.g., mRNA) typically comprises a plurality of nucleotides. A nucleotide includes a nitrogenous base, a five-carbon sugar (ribose or deoxyribose), and at least one phosphate group. Nucleotides include nucleoside monophosphates, nucleoside diphosphates, and nucleoside triphosphates. A nucleoside monophosphate (NMP) includes a nucleobase linked to a ribose and a single phosphate; a nucleoside diphosphate (NDP) includes a nucleobase linked to a ribose and two phosphates; and a nucleoside triphosphate (NTP) includes a nucleobase linked to a ribose and three phosphates. Nucleotide analogs are compounds that have the general structure of a nucleotide or are structurally similar to a nucleotide. Nucleotide analogs, for example, include an analog of the nucleobase, an analog of the sugar and/or an analog of the phosphate group(s) of a nucleotide.

A nucleoside includes a nitrogenous base and a 5-carbon sugar. Thus, a nucleoside plus a phosphate group yields a nucleotide. Nucleoside analogs are compounds that have the general structure of a nucleoside or are structurally similar to a nucleoside. Nucleoside analogs, for example, include an analog of the nucleobase and/or an analog of the sugar of a nucleoside.

It should be understood that the term “nucleotide” includes naturally-occurring nucleotides, synthetic nucleotides and modified nucleotides, unless indicated otherwise. Examples of naturally-occurring nucleotides used for the production of RNA, e.g., in an IVT reaction, as provided herein include adenosine triphosphate (ATP), guanosine triphosphate (GTP), cytidine triphosphate (CTP), uridine triphosphate (UTP), and 5-methyluridine triphosphate (m⁵UTP). In some embodiments, adenosine diphosphate (ADP), guanosine diphosphate (GDP), cytidine diphosphate (CDP), and/or uridine diphosphate (UDP) are used.

Examples of nucleotide analogs include, but are not limited to, antiviral nucleotide analogs, phosphate analogs (soluble or immobilized, hydrolyzable or non-hydrolyzable), dinucleotide, trinucleotide, tetranucleotide, e.g., a cap analog, or a precursor/substrate for enzymatic capping (vaccinia or ligase), a nucleotide labeled with a functional group to facilitate ligation/conjugation of cap or 5′ moiety (IRES), a nucleotide labeled with a 5′ PO₄ to facilitate ligation of cap or 5′ moiety, or a nucleotide labeled with a functional group/protecting group that can be chemically or enzymatically cleaved. Examples of antiviral nucleotide/nucleoside analogs include, but are not limited, to Ganciclovir, Entecavir, Telbivudine, Vidarabine and Cidofovir.

Modified nucleotides may include modified nucleobases. For example, an RNA transcript (e.g., mRNA transcript) may include a modified nucleobase selected from pseudouridine (W), 1-methylpseudouridine (m1ψ), 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine. In some embodiments, an RNA transcript (e.g., mRNA transcript) includes a combination of at least two (e.g., 2, 3, 4 or more) of the foregoing modified nucleobases.

### In Vitro Transcription

Aspects of the disclosure relate to methods of producing (e.g., synthesizing) an RNA transcript (e.g., mRNA transcript) comprising contacting a DNA template (e.g., an input DNA) with an RNA polymerase (e.g., a T7 RNA polymerase, a T7 RNA polymerase variant, etc.) under conditions that result in the production of the RNA transcript. This process is referred to as “in vitro transcription” or “IVT”. IVT conditions typically require a purified DNA template containing a promoter, nucleoside triphosphates, a buffer system that includes dithiothreitol (DTT) and magnesium ions, and an RNA polymerase. The exact conditions used in the transcription reaction depend on the amount of RNA needed for a specific application. Typical IVT reactions are performed by incubating a DNA template with an RNA polymerase and nucleoside triphosphates, including GTP, ATP, CTP, and UTP (or nucleotide analogs) in a transcription buffer. An RNA transcript having a 5′ terminal guanosine triphosphate is produced from this reaction.

In some embodiments, an IVT reaction uses an RNA polymerase selected from the group consisting of T7 RNA polymerase, T3 RNA polymerase, K11 RNA polymerase, and SP6 RNA polymerase. In some embodiments, an IVT reaction uses a T3 RNA polymerase. In some embodiments, an IVT reaction uses an SP6 RNA polymerase. In some embodiments, an IVT reaction uses a K11 RNA polymerase. In some embodiments, an IVT reaction uses a T7 RNA polymerase. In some embodiments, a wild-type T7 polymerase is used in an IVT reaction. In some embodiments, a mutant T7 polymerase is used in an IVT reaction. In some embodiments, a T7 RNA polymerase variant comprises an amino acid sequence that shares at least 50%, 60%, 70%, 80%, 90%, 95%, or 99% identity with a wild-type T7 (WT T7) polymerase. In some embodiments, the T7 polymerase variant is a T7 polymerase variant described by International Application Publication Number WO2019/036682 or WO2020/172239, the entire contents of each of which are incorporated herein by reference. In some embodiments, a T7 RNA polymerase variant comprises an amino acid sequence with at least 50%, 60%, 70%, 80%, 90%, 95%, or 99% sequence identity to the amino acid sequence of SEQ ID NO: 1. In some embodiments, the T7 RNA polymerase variant comprises the amino acid sequence of SEQ ID NO: 1. T7 RNA polymerase variants with one or more mutations relative to WT T7 RNA polymerase have several advantages in IVT reactions, including improved speed, fidelity, and reduced production of double-stranded RNA (dsRNA) transcripts. Double-stranded RNA transcripts, in which at least a portion of an RNA transcript is hybridized to another RNA molecule, elicit an innate immune response when introduced into a cell, causing degradation of both strands of a dsRNA. Minimizing the formation of dsRNA transcripts during IVT enables the production of less immunogenic, and thus more stable, RNA compositions. In some embodiments, the concentration of double-stranded RNA in a composition comprising RNA is 5% (% w/w) or less, 4% or less, 3% or less, 2.5% or less, 2% or less, 1.75% or less, 1.5% or less, 1.25% or less, 1% or less, 0.9% or less, 0.8% or less, 0.7% or less, 0.6% or less, 0.5% or less, 0.4% or less, 0.3% or less, 0.25% or less, 0.2% or less, 0.175% or less, 0.15% or less, 0.125% or less, or 0.1% or less. In some embodiments, the concentration of double-stranded RNA in a composition comprising RNA is 0.05% (% w/w) or less, 0.04% or less, 0.03% or less, 0.02% or less, or 0.01% or less. Methods of measuring the presence and/or amount of dsRNA in a composition are known in the art. Non-limiting examples of methods for measuring dsRNA content of a sample include ELISAs and immunoblotting using antibodies specific to dsRNA. Additionally, the total mass of RNA in a sample can be measured using techniques such as spectroscopy (NanoDrop), qRT-PCR, and/or ddPCR, and the mass of dsRNA can be measured using an intercalating agent that fluoresces when bound to dsRNA, such as acridine orange, with the dsRNA concentration being calculated by division. In some embodiments, the concentration of dsRNA in a composition refers to the mass of RNA nucleotides that are part of a double-stranded RNA:RNA hybrid, with other unhybridized nucleotides from either RNA in the hybrid not contributing to the amount of dsRNA in a composition. In other embodiments, the concentration of dsRNA in a sample refers to the concentration of RNA molecules containing nucleotides that are part of an RNA:RNA hybrid. In some embodiments, the RNA polymerase (e.g., T7 RNA polymerase or T7 RNA polymerase variant) is present in a reaction (e.g., an IVT reaction) at a concentration of 0.01 mg/ml to 1 mg/ml. For example, the RNA polymerase may be present in a reaction at a concentration of 0.01 mg/mL, 0.05 mg/ml, 0.1 mg/ml, 0.5 mg/ml or 1.0 mg/ml.

The “percent identity,” “sequence identity,” “% identity,” or “% sequence identity” (as they may be interchangeably used herein) of two sequences (e.g., nucleic acid or amino acid) refers to a quantitative measurement of the similarity between two sequences (e.g., nucleic acid or amino acid). Percent identity can be determined using the algorithms of Karlin and Altschul, Proc. Natl. Acad. Sci. USA 87:2264-68, 1990, modified as in Karlin and Altschul, Proc. Natl. Acad. Sci. USA 90:5873-77, 1993. Such algorithms are incorporated into the NBLAST and XBLAST programs (version 2.0) of Altschul et al., J. Mol. Biol. 215:403-10, 1990. BLAST protein searches can be performed with the XBLAST program, score=50, word length=3, to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used. When a percent identity is stated, or a range thereof (e.g., at least, more than, etc.), unless otherwise specified, the endpoints shall be inclusive and the range (e.g., at least 70% identity) shall include all ranges within the cited range.

The input deoxyribonucleic acid (DNA) serves as a nucleic acid template for RNA polymerase. A DNA template may include a polynucleotide encoding a polypeptide of interest (e.g., an antigenic polypeptide). A DNA template, in some embodiments, includes an RNA polymerase promoter (e.g., a T7 RNA polymerase promoter) located 5′ from and operably linked to polynucleotide encoding a polypeptide of interest. A DNA template may also include a nucleotide sequence encoding a polyadenylation (polyA) region located at the 3′ end of the gene of interest. In some embodiments, an input DNA comprises plasmid DNA (pDNA). As used herein, “plasmid DNA” or “pDNA” refers to an extrachromosomal DNA molecule that is physically separated from chromosomal DNA in a cell and can replicate independently. In some embodiments, plasmid DNA is isolated from a cell (e.g., as a plasmid DNA preparation). In some embodiments, plasmid DNA comprises an origin of replication, which may contain one or more heterologous nucleic acids, for example nucleic acids encoding therapeutic proteins that may serve as a template for RNA polymerase. Plasmid DNA may be circularized or linear (e.g., plasmid DNA that has been linearized by a restriction enzyme digest).

Some embodiments comprise performing a co-IVT reaction that includes multiple input DNAs (or populations of input DNAs). In some embodiments, each input DNA (e.g., population of input DNA molecules) in a co-IVT reaction is obtained from a different source (e.g., synthesized separately, for example in different cells or populations of cells). In some embodiments, each input DNA (e.g., population of input DNA) is obtained from a different bacterial cell or population of bacterial cells. For example, in a co-IVT reaction having three populations of input DNAs, the first input DNA is produced in bacterial cell population A, the second input DNA is produced in bacterial cell population B, and the third input DNA is produced in bacterial population C, where each of A, B, and C are not the same bacterial culture (e.g., co-cultured in the same container or plate). In another example, two input DNAs obtained from different sources are i) chemically synthesized in separate synthesis reactions, or ii) produced by separate amplification (e.g., polymerase chain reactions (PCR reactions)). An RNA transcript, in some embodiments, is the product of an IVT reaction. An RNA transcript, in some embodiments, is a messenger RNA (mRNA) that includes a nucleotide sequence encoding a polypeptide of interest (e.g., a therapeutic protein or therapeutic peptide) linked to a polyA tail. In some embodiments, the mRNA is modified mRNA (mmRNA), which includes at least one modified nucleotide. In some embodiments, an RNA transcript produced by IVT is further modified by circularization, in which two non-adjacent nucleotides (e.g., 5′ and 3′ terminal nucleotides) of a linear RNA are ligated to produce a circular RNA with no terminal nucleotides.

The nucleoside triphosphates (NTPs) as provided herein may comprise unmodified or modified ATP, modified or unmodified UTP, modified or unmodified GTP, and/or modified or unmodified CTP. In some embodiments, NTPs of an IVT reaction comprise unmodified ATP. In some embodiments, NTPs of an IVT reaction comprise modified ATP. In some embodiments, NTPs of an IVT reaction comprise unmodified UTP. In some embodiments, NTPs of an IVT reaction comprise modified UTP. In some embodiments, NTPs of an IVT reaction comprise unmodified GTP. In some embodiments, NTPs of an IVT reaction comprise modified GTP. In some embodiments, NTPs of an IVT reaction comprise unmodified CTP. In some embodiments, NTPs of an IVT reaction comprise modified CTP.

The composition of NTPs in an IVT reaction may also vary. In some embodiments, each NTP in an IVT reaction is present in an equimolar amount. In some embodiments, each NTP in an IVT reaction is present in non-equimolar amounts. For example, ATP may be used in excess of GTP, CTP and UTP. As a non-limiting example, an IVT reaction may include 7.5 millimolar GTP, 7.5 millimolar CTP, 7.5 millimolar UTP, and 3.75 millimolar ATP. In some embodiments, the molar ratio of G:C:U:A is 2:1:0.5:1. In some embodiments, the molar ratio of G:C:U:A is 1:1:0.7:1. In some embodiments, the molar ratio of G:C:A:U is 1:1:1:1. The same IVT reaction may include 3.75 millimolar cap analog (e.g., trinucleotide cap or tetranucleotide cap). In some embodiments, the molar ratio of the cap to any of G, C, U, or A is 1:1. In some embodiments, the molar ratio of G:C:U:A:cap is 1:1:1:0.5:0.5. In some embodiments, the molar ratio of G:C:U:A:cap is 1:1:0.5:1:0.5. In some embodiments, the molar ratio of G:C:U:A:cap is 1:0.5:1:1:0.5. In some embodiments, the molar ratio of G:C:U:A:cap is 0.5:1:1:1:0.5. In some embodiments, the amount of NTPs in a IVT reaction is calculated empirically. For example, the rate of consumption for each NTP in an IVT reaction may be empirically determined for each individual input DNA, and then balanced ratios of NTPs based on those individual NTP consumption rates may be added to a IVT comprising multiple of the input DNAs.

In some embodiments, the IVT reaction mixture comprises one or more modified nucleoside triphosphates. In some embodiments, the IVT reaction mixture comprises one or more modified nucleoside triphosphates selected from the group consisting of N6-methyladenosine triphosphate, pseudouridine (ψ) triphosphate, 1-methylpseudouridine (m¹ψ) triphosphate, 5-methoxyuridine (mo⁵U) triphosphate, 5-methylcytidine (m⁵C) triphosphate, α-thio-guanosine triphosphate, and α-thio-adenosine triphosphate. In some embodiments, the IVT reaction mixture comprises N6-methyladenosine triphosphate. In some embodiments, the IVT reaction mixture comprises pseudouridine triphosphate. In some embodiments, the IVT reaction mixture comprises 1-methylpseudouridine triphosphate. In some embodiments, the concentration of modified nucleoside triphosphates in the reaction mixture is about 0.1% to about 100%, about 0.5% to about 75%, about 1% to about 50%, or about 2% to about 25%. In some embodiments, the concentration of modified nucleoside triphosphates is about 1%, about 2%, about 3%, about 4%, about 5%, about 6%, about 7%, about 8%, about 9%, about 10%, about 15%, about 20%, or about 25%.

In some embodiments, an RNA transcript (e.g., mRNA transcript) includes a modified nucleobase selected from pseudouridine (W), 1-methylpseudouridine (myl), 5-methoxyuridine (mo⁵U), 5-methylcytidine (m⁵C), α-thio-guanosine and α-thio-adenosine. In some embodiments, an RNA transcript (e.g., mRNA transcript) includes a combination of at least two (e.g., 2, 3, 4 or more) of the foregoing modified nucleobases.

In some embodiments, an RNA transcript (e.g., mRNA transcript) includes pseudouridine (ψ). In some embodiments, an RNA transcript (e.g., mRNA transcript) includes 1-methylpseudouridine (m¹ψ). In some embodiments, an RNA transcript (e.g., mRNA transcript) includes 5-methoxyuridine (mo⁵U). In some embodiments, an RNA transcript (e.g., mRNA transcript) includes 5-methylcytidine (m⁵C). In some embodiments, an RNA transcript (e.g., mRNA transcript) includes α-thio-guanosine. In some embodiments, an RNA transcript (e.g., mRNA transcript) includes α-thio-adenosine.

In some embodiments, the polynucleotide (e.g., RNA polynucleotide, such as mRNA polynucleotide) is uniformly modified (e.g., fully modified, modified throughout the entire sequence) for a particular modification. For example, a polynucleotide can be uniformly modified with 1-methylpseudouridine (m1ψ), meaning that all uridine residues in the mRNA sequence are replaced with 1-methylpseudouridine (m¹ψ). Similarly, a polynucleotide can be uniformly modified for any type of nucleoside residue present in the sequence by replacement with a modified residue such as any of those set forth above. Alternatively, the polynucleotide (e.g., RNA polynucleotide, such as mRNA polynucleotide) may not be uniformly modified (e.g., partially modified, part of the sequence is modified). Each possibility represents a separate embodiment. In some embodiments, modified nucleotides are included in an IVT mixture, and are incorporated randomly during transcription, such that the RNA contains a mixture of modified nucleotides and unmodified nucleotides.

The buffer system of an IVT reaction mixture may vary. In some embodiments, the buffer system contains Tris. The concentration of tris used in an IVT reaction, for example, may be at least 10 mM, at least 20 mM, at least 30 mM, at least 40 mM, at least 50 mM, at least 60 mM, at least 70 mM, at least 80 mM, at least 90 mM, at least 100 mM or at least 110 mM phosphate. In some embodiments, the concentration of phosphate is 20-60 mM or 10-100 mM. In some embodiments, the buffer system contains dithiothreitol (DTT). The concentration of DTT used in an IVT reaction, for example, may be at least 1 mM, at least 5 mM, or at least 50 mM. In some embodiments, the concentration of DTT used in an IVT reaction is 1-50 mM or 5-50 mM. In some embodiments, the concentration of DTT used in an IVT reaction is 5 mM. In some embodiments, the buffer system contains magnesium. In some embodiments, the molar ratio of NTP to magnesium ions (Mg²⁺; e.g., MgCl₂) present in an IVT reaction is 1:1 to 1:5. For example, the molar ratio of NTP to magnesium ions may be 1:0.25, 1:0.5, 1:1, 1:2, 1:3, 1:4 or 1:5.

In some embodiments, the molar ratio of NTP to magnesium ions (Mg²⁺; e.g., MgCl₂) present in an IVT reaction is 1:1 to 1:5. For example, the molar ratio of NTP to magnesium ions may be 1:1, 1:2, 1:3, 1:4 or 1:5.

In some embodiments, the buffer system contains Tris-HCl, spermidine (e.g., at a concentration of 1-30 mM), TRITON® X-100 (polyethylene glycol p-(1,1,3,3-tetramethylbutyl)-phenyl ether) and/or polyethylene glycol (PEG).

In some embodiments, IVT methods further comprise a step of separating (e.g., purifying) in vitro transcription products (e.g., mRNA) from other reaction components. In some embodiments, the separating comprises performing chromatography on the IVT reaction mixture. In some embodiments, the method comprises reverse phase chromatography. In some embodiments, the method comprises reverse phase column chromatography. In some embodiments, the chromatography comprises size-based (e.g., length-based) chromatography. In some embodiments, the method comprises size exclusion chromatography. In some embodiments, the chromatography comprises oligo-dT chromatography.

### Untranslated Regions

Untranslated regions (UTRs) are sections of a nucleic acid before a start codon (5′ UTR) and after a stop codon (3′ UTR) that are not translated. In some embodiments, a nucleic acid (e.g., a ribonucleic acid (RNA), e.g., a messenger RNA (mRNA)) comprising an open reading frame (ORF) encoding one or more proteins or peptides further comprises one or more UTR (e.g., a 5′ UTR or functional fragment thereof, a 3′ UTR or functional fragment thereof, or a combination thereof).

A UTR can be homologous or heterologous to the coding region in a nucleic acid. In some embodiments, the UTR is homologous to the ORF encoding the one or more peptide epitopes. In some embodiments, the UTR is heterologous to the ORF encoding the one or more peptide epitopes. In some embodiments, the nucleic acid comprises two or more 5′ UTRs or functional fragments thereof, each of which have the same or different nucleotide sequences. In some embodiments, the nucleic acid comprises two or more 3′ UTRs or functional fragments thereof, each of which have the same or different nucleotide sequences.

In some embodiments, the 5′ UTR or functional fragment thereof, 3′ UTR or functional fragment thereof, or any combination thereof is sequence optimized.

In some embodiments, the 5′ UTR or functional fragment thereof, 3′ UTR or functional fragment thereof, or any combination thereof comprises at least one chemically modified nucleobase, e.g., 5-methoxyuracil.

UTRs can have features that provide a regulatory role, e.g., increased or decreased stability, localization, and/or translation efficiency. A nucleic acid comprising a UTR can be administered to a cell, tissue, or organism, and one or more regulatory features can be measured using routine methods. In some embodiments, a functional fragment of a 5′ UTR or 3′ UTR comprises one or more regulatory features of a full length 5′ or 3′ UTR, respectively.

Natural 5′ UTRs bear features that play roles in translation initiation. They harbor signatures like Kozak sequences that are commonly known to be involved in the process by which the ribosome initiates translation of many genes. 5′ UTRs also have been known to form secondary structures that are involved in elongation factor binding.

By engineering the features typically found in abundantly expressed genes of specific target organs, one can enhance the stability and protein production of a nucleic acid. For example, introduction of 5′ UTR of liver-expressed mRNA, such as albumin, serum amyloid A, Apolipoprotein A/B/E, transferrin, alpha fetoprotein, erythropoietin, or Factor VIII, can enhance expression of nucleic acids in hepatic cell lines or liver. Likewise, use of 5′ UTRs from other tissue-specific mRNA to improve expression in that tissue is possible for muscle (e.g., MyoD, Myosin, Myoglobin, Myogenin, Herculin), for endothelial cells (e.g., Tie-1, CD36), for myeloid cells (e.g., C/EBP, AML1, G-CSF, GM-CSF, CD11b, MSR, Fr-1, i-NOS), for leukocytes (e.g., CD45, CD18), for adipose tissue (e.g., CD36, GLUT4, ACRP30, adiponectin), and for lung epithelial cells (e.g., SP-A/B/C/D).

In some embodiments, UTRs are selected from a family of transcripts whose proteins share a common function, structure, feature, or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new nucleic acid.

In some embodiments, the 5′ UTR and the 3′ UTR can be heterologous. In some embodiments, the 5′ UTR can be derived from a different species than the 3′ UTR. In some embodiments, the 3′ UTR can be derived from a different species than the 5′ UTR.

International Patent Application No. PCT/US2014/021522 (Publ. No. WO/2014/164253) provides a listing of exemplary UTRs that may be utilized in the nucleic acids as flanking regions to an ORF. This publication is incorporated by reference herein for this purpose.

Additional exemplary UTRs that may be utilized in the nucleic acids include, but are not limited to, one or more 5′ UTRs and/or 3′ UTRs derived from the nucleic acid sequence of: a globin, such as an α- or β-globin (e.g., a Xenopus, mouse, rabbit, or human globin); a strong Kozak translational initiation signal; a CYBA (e.g., human cytochrome b-245 a polypeptide); an albumin (e.g., human albumin7); a HSD17B4 (hydroxysteroid (17-0) dehydrogenase); a virus (e.g., a tobacco etch virus (TEV), a Venezuelan equine encephalitis virus (VEEV), a Dengue virus, a cytomegalovirus (CMV; e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human α or β actin); a GAPDH; a tubulin; a histone; a citric acid cycle enzyme; a topoisomerase (e.g., a 5′ UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the β subunit of mitochondrial H⁺-ATP synthase); a growth hormone (e.g., bovine (bGH) or human (hGH)); an elongation factor (e.g., elongation factor 1 α1 (EEF1A1)); a manganese superoxide dismutase (MnSOD); a myocyte enhancer factor 2A (MEF2A); a 1-F1-ATPase, a creatine kinase, a myoglobin, a granulocyte-colony stimulating factor (G-CSF); a collagen (e.g., collagen type I, alpha 2 (Col1A2), collagen type I, alpha 1 (Col1A1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nnt1); calreticulin (Calr); a procollagen-lysine, 2-oxoglutarate 5-dioxygenase 1 (Plod1); and a nucleobindin (e.g., Nucb1).

In some embodiments, the 5′ UTR is selected from the group consisting of a β-globin 5′ UTR; a 5′ UTR containing a strong Kozak translational initiation signal; a cytochrome b-245 a polypeptide (CYBA) 5′ UTR; a hydroxysteroid (17-β) dehydrogenase (HSD17B4) 5′ UTR; a Tobacco etch virus (TEV) 5′ UTR; a Venezuelen equine encephalitis virus (TEEV) 5′ UTR; a 5′ proximal open reading frame of rubella virus (RV) RNA encoding nonstructural proteins; a Dengue virus (DEN) 5′ UTR; a heat shock protein 70 (Hsp70) 5′ UTR; a eIF4G 5′ UTR; a GLUT1 5′ UTR; functional fragments thereof and any combination thereof.

In some embodiments, the 3′ UTR is selected from the group consisting of a β-globin 3′ UTR; a CYBA 3′ UTR; an albumin 3′ UTR; a growth hormone (GH) 3′ UTR; a VEEV 3′ UTR; a hepatitis B virus (HBV) 3′ UTR; α-globin 3′ UTR; a DEN 3′ UTR; a PAV barley yellow dwarf virus (BYDV-PAV) 3′ UTR; an elongation factor 1 al (EEF1A1) 3′ UTR; a manganese superoxide dismutase (MnSOD) 3′ UTR; a R subunit of mitochondrial H(+)-ATP synthase ((3-mRNA) 3′ UTR; a GLUT1 3′ UTR; a MEF2A 3′ UTR; a 0-F1-ATPase 3′ UTR; functional fragments thereof and combinations thereof.

Wild-type UTRs derived from any gene or mRNA can be incorporated into the nucleic acids. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more non-synthetic UTRs. See, e.g., Mandal and Rossi, Nat. Protoc. 2013 8(3):568-82, and sequences available at www.addgene.org, the contents of each are incorporated herein by reference in their entirety. UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs.

In some embodiments, the nucleic acid may comprise multiple UTRs, e.g., a double, a triple or a quadruple 5′ UTR or 3′ UTR. For example, a double UTR comprises two copies of the same UTR either in series or substantially in series. For example, a double beta-globin 3′ UTR can be used (see, for example, US2010/0129877, the contents of which are incorporated herein by reference for this purpose).

The nucleic acids can comprise combinations of features. For example, the ORF can be flanked by a 5′ UTR that comprises a strong Kozak translational initiation signal and/or a 3′ UTR comprising an oligo(dT) sequence for templated addition of a polyA tail. A 5′ UTR can comprise a first nucleic acid fragment and a second nucleic acid fragment from the same and/or different UTRs (see, e.g., US2010/0293625, herein incorporated by reference in its entirety for this purpose).

Other non-UTR sequences can be used as regions or subregions within the nucleic acids. For example, introns or portions of intron sequences can be incorporated into the nucleic acids. Incorporation of intronic sequences can increase protein production as well as nucleic acid expression levels. In some embodiments, the nucleic acid comprises an internal ribosome entry site (IRES) instead of or in addition to a UTR (see, e.g., Yakubov et al., Biochem. Biophys. Res. Commun. 2010 394(1):189-193, the contents of which are incorporated herein by reference in their entirety). In some embodiments, the nucleic acid comprises an IRES instead of a 5′ UTR sequence. In some embodiments, the nucleic acid comprises an IRES that is located between a 5′ UTR and an open reading frame. In some embodiments, the nucleic acid comprises an ORF encoding a viral capsid sequence. In some embodiments, the nucleic acid comprises a synthetic 5′ UTR in combination with a non-synthetic 3′ UTR.

In some embodiments, the UTR can also include at least one translation enhancer nucleic acid, translation enhancer element, or translational enhancer elements (collectively, “TEE,” which refers to nucleic acid sequences that increase the amount of polypeptide or protein produced from a polynucleotide. As a non-limiting example, the TEE can include those described in US2009/0226470, incorporated herein by reference in its entirety for this purpose, and others known in the art. As a non-limiting example, the TEE can be located between the transcription promoter and the start codon. In some embodiments, the 5′ UTR comprises a TEE. In one aspect, a TEE is a conserved element in a UTR that can promote translational activity of a nucleic acid such as, but not limited to, cap-dependent or cap-independent translation. In one non-limiting example, the TEE comprises the TEE sequence in the 5′-leader of the Gtx homeodomain protein. See, e.g., Chappell et al., PNAS. 2004. 101:9590-9594, incorporated herein by reference in its entirety for this purpose.

### Poly(A) Tails

Aspects of the disclosure relate to methods of producing RNAs containing one or more polyA tails. A “polyA tail” is a region of mRNA that is downstream, e.g., directly downstream (i.e., 3′), from the open reading frame and/or the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo, etc.) the poly(A) tail functions to protect mRNA from enzymatic degradation, e.g., in the cytoplasm, and aids in transcription termination, export of the mRNA from the nucleus, and translation.

As used herein, “polyA-tailing efficiency” refers to the amount (e.g., expressed as a percentage) of mRNAs having polyA tail that are produced by an IVT reaction using an input DNA relative to the total number of mRNAs produced in the IVT reaction using the input DNA. The polyA-tailing efficiency of an IVT reaction may vary, for example depending upon the RNA polymerase used, amount or purity of input DNA used, etc. In some embodiments, the polyA-tailing efficiency of an IVT reaction is greater than 85%, 90%, 95%, or 99.9%. Methods of calculating polyA-tailing efficiency are known, for example by determining the amount of polyA tail-containing mRNA relative to total mRNA produced in an IVT reaction by column chromatography (e.g., oligo-dT chromatography).

In some embodiments, at least 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 99.9% of RNAs in an RNA composition produced by a method described herein comprise a polyA tail. In some embodiments, at least 85%, 86%, 87%, 88%, 89%, 90%, 91%, 92%, 93%, 94%, 95%, 96%, 97%, 98%, 99%, or 99.9% of each RNA in an RNA composition produced by a method described herein comprise a polyA tail. The efficiency (e.g., percentage of polyA tail-containing RNAs in an RNA composition may be measured i) after the IVT reaction and before purification, or ii) after the RNA composition has been purified (e.g., by chromatography, such as oligo-dT chromatography).

Unique polyA tail lengths provide certain advantages to the nucleic acids. Generally, the length of a polyA tail, when present, is greater than 30 nucleotides in length. In another embodiment, the polyA tail is greater than 35 nucleotides in length (e.g., at least or greater than about 15, 20, 25, 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, or 3,000 nucleotides).

In some embodiments, the polyA tail is designed relative to the length of the overall nucleic acid or the length of a particular region of the nucleic acid. This design can be based on the length of a coding region, the length of a particular feature or region or based on the length of the ultimate product expressed from the nucleic acids.

In this context, the polyA tail can be 10, 20, 30, 40, 50, 60, 70, 80, 90, or 100% greater in length than the nucleic acid or feature thereof. The polyA tail can also be designed as a fraction of the nucleic acid to which it belongs. In this context, the polyA tail can be 10, 20, 30, 40, 50, 60, 70, 80, or 90% or more of the total length of the construct, a construct region or the total length of the construct minus the polyA tail. Further, engineered binding sites and conjugation of nucleic acids for PolyA-binding protein can enhance expression.

### Protease Digestion

Some aspects of the disclosure relate to methods of purifying in vitro transcribed mRNA by digesting one or more proteins used in IVT by introducing a protease into an IVT mixture after an RNA has been transcribed, and isolating the mRNA from the mixture. Exemplary IVT enzymes include RNA polymerases, which transcribe the RNA from a DNA template, capping enzymes, which add a 5′ cap or cap analog to the transcribed RNA, and polyadenylating enzymes, which add or extend the polyA tail of an RNA. Additionally, DNases may be introduced to an IVT mixture to digest DNA templates in the mixture, and proteases added later may also digest the DNases. Proteases are enzymes that catalyze the breakdown of proteins into smaller protein fragments, such as smaller polypeptides, oligopeptides, or individual amino acids. Generally, proteases catalyze hydrolysis of the peptide bonds that connect amino acids in polypeptide chains, with hydrolysis of a peptide bond resulting in the release of two polypeptides, two amino acids, or an amino acid and a polypeptide, depending on the site of cleavage. Exopeptidases are proteases that catalyze the peptide bond between a terminal amino acid in a polypeptide and an adjacent amino acid, resulting in the release of the terminal amino acid from the rest of the polypeptide. Endopeptidases are proteases that catalyze the peptide bond between internal amino acids in a polypeptide, releasing two separate polypeptides or oligopeptides. Endopeptidases may vary in specificity, cleaving more readily before or after certain amino acid residues. For example, trypsin readily cleaves after arginine or lysine, unless the arginine or lysine is followed by a proline. Cleaving “after” a first amino acid refers to cleavage of the peptide bond that connects the first amino acid to the next amino acid in a protein, with the protein being described by an amino acid sequence listing amino acids from the N-terminus to the C-terminus.

In some embodiments of the methods provided herein, the protease introduced into the mixture is selected from the group consisting of proteinase K, Lys-C, trypsin, TPCK-treated trypsin, chymotrypsin, α-lytic protease, and endoproteinase AspN. In some embodiments, the protease is a serine protease, such as proteinase K. Proteinase K is an endopeptidase with broad specificity that cleaves the peptide bond adjacent to the carboxyl group of aliphatic and aromatic amino acids. Exemplary amino acids that can be serve as substrates for cleavage by proteinase K include alanine, glycine, isoleucine, leucine, proline, valine, tryptophan, tyrosine, and phenylalanine. An example of a DNA sequence encoding proteinase K is given by Accession No. X14688, which is reproduced below as SEQ ID NO: 2. An example of an amino acid sequence of proteinase K is given by Accession No. P06873, which is reproduced below as SEQ ID NO: 3. In some embodiments, the protease comprises an amino acid sequence with at least 90%, at least 91%, at least 92%, at least 93%, at least 94%, at least 95%, at least 96%, at least 97%, at least 98%, at least 99%, or up to 100% sequence identity to the amino acid sequence of SEQ ID NO: 3. In some embodiments, the protease comprises an amino acid sequence with at least 90% sequence identity to the amino acid sequence of SEQ ID NO: 3. In some embodiments, the protease comprises an amino acid sequence with at least 95% sequence identity to the amino acid sequence of SEQ ID NO: 3. In some embodiments, the protease comprises an amino acid sequence with at least 97% sequence identity to the amino acid sequence of SEQ ID NO: 3. In some embodiments, the protease comprises the amino acid sequence of SEQ ID NO: 3. In some embodiments, the proteinase K is a thermolabile proteinase K. Thermolabile refers to a molecule, such as a protein, that can be denatured by exposure to heat.

In some embodiments, after the protease is introduced into the IVT mixture, the concentration of the protease in the mixture is about 0.1 Units/mL to about 100 Units/mL. With respect to a protein, a “Unit” (“U”) refers to an amount of the protein that is capable of performing a specific function in a given amount of time. For example, one unit of proteinase K is defined as the amount of enzyme required to liberate folin-positive amino acids and peptides corresponding to 1 μmol of tyrosine in 1 minute at 37° C. in a total reaction volume of 250 μL. See, e.g., Anson. J Gen Physiol. 1938. 22(1):79-89. In some embodiments, the concentration of protease in the mixture is about 0.2 to about 50 Units/mL, about 0.3 to about 25 Units/mL, about 0.4 to about 10 Units/mL, about 0.5 to about 5 Units/mL, about 0.5 to about 3 Units/mL, about 0.5 to about 2 Units/mL, or about 0.5 to about 1 Unit/mL. In some embodiments, the concentration of protease in the mixture is about 0.1 to about 2 Units/mL. In some embodiments, the concentration of protease in the mixture is about 1 to about 10 Units/mL. In some embodiments, the concentration of protease in the mixture is about 10 to about 100 Units/mL.

In some embodiments, the amount of the protease in the mixture during the protease digestion step, relative to the amount of RNA polymerase in the mixture, is at least 1:1,000,000 (1 Unit protease: 1,000,000 μmol RNA polymerase). In some embodiments, the protease:RNA polymerase concentration in the mixture is about 1:10 to about 1:100, about 1:100 to about 1:1,000, about 1:1,000 to about 1:10,000, about 1:10,000 to about 1:100,000, or about 1:100,000 to about 1:1,000,000. In some embodiments, the protease:RNA polymerase concentration in the mixture is about 1:1,000 to about 1:50,000. In some embodiments, the amount of the protease in the mixture during the protease digestion step, relative to the amount of other proteins in the mixture, is at least 1:1,000,000 (1 Unit protease: 1,000,000 μmol other proteins). In some embodiments, the protease:protein concentration in the mixture is about 1:10 to about 1:100, about 1:100 to about 1:1,000, about 1:1,000 to about 1:10,000, about 1:10,000 to about 1:100,000, or about 1:100,000 to about 1:1,000,000. In some embodiments, the protease:protein concentration in the mixture is about 1:1,000 to about 1:50,000.

In some embodiments, the step of protease digestion is conducted at about 37° C. In some embodiments, the protease digestion step is conducted at a temperature of 70° C. or lower, 60° C. or lower, 50° C. or lower, or 40° C. or lower. In some embodiments, the step of protease digestion is conducted for about 10 minutes to about 6 hours. In some embodiments, the step of protease digestion is conducted for at least 10 minutes, at least 15 minutes, at least 20 minutes, at least 30 minutes, at least 40 minutes, at least 50 minutes, or at least 60 minutes. In some embodiments, the step of protease digestion is conducted for at least 1 hour, at least 2 hours, at least 3 hours, at least 4 hours, or at least 5 hours. In some embodiments, the step of protease digestion is conducted for about 15 minutes. In some embodiments, the step of protease digestion is conducted for about 30 minutes. In some embodiments, the step of protease digestion is conducted for about 45 minutes. In some embodiments, the step of protease digestion is conducted for about 60 minutes.

In some embodiments, the IVT mixture comprises one or more cations. In some embodiments, one or more cations are added to the IVT mixture during or after IVT. The presence and concentration of cations can affect the activity of proteases. For example, some proteases are more active in the presence of divalent cations, such as magnesium (Mg²⁺) ions. Adding magnesium or other cations to a mixture can thus improve the efficiency of protease digestion, thereby allowing for removal of more residual proteins from an IVT mixture to produce a more pure RNA composition. In some embodiments, the cation present in or added to the mixture is a magnesium ion. In some embodiments, the cation present in or added to the mixture is a calcium ion. In some embodiments, the concentration of cations in the mixture during protease digestion is about 10 mM to about 100 mM. In some embodiments, the concentration of cations is about 1 mM to about 5 mM, about 5 mM to about 10 mM, about 10 mM to about 20 mM, about 20 mM to about 30 mM, about 30 mM to about 40 mM, about 40 mM to about 50 mM, about 50 mM to about 60 mM, about 60 mM to about 70 mM, about 70 mM to about 80 mM, about 80 mM to about 90 mM, or about 90 to about 100 mM. In some embodiments, the concentration of cations is about 10 mM. In some embodiments, the concentration of cations is about 20 mM. In some embodiments, the concentration of cations is about 30 mM. In some embodiments, the concentration of magnesium ions in the mixture during protease digestion is about 10 mM to about 100 mM. In some embodiments, the concentration of magnesium ions is about 1 mM to about 5 mM, about 5 mM to about 10 mM, about 10 mM to about 20 mM, about 20 mM to about 30 mM, about 30 mM to about 40 mM, about 40 mM to about 50 mM, about 50 mM to about 60 mM, about 60 mM to about 70 mM, about 70 mM to about 80 mM, about 80 mM to about 90 mM, or about 90 to about 100 mM. In some embodiments, the concentration of magnesium ions is about 10 mM. In some embodiments, the concentration of magnesium ions is about 20 mM. In some embodiments, the concentration of magnesium ions is about 30 mM.

In some embodiments, the method further comprises introducing a DNase to the IVT mixture and incubating the mixture for a period of time sufficient for the DNase to cleave one or more DNAs in the mixture to produce one or more DNA fragments. Following in vitro transcription, digestion of DNA templates with DNase and protease digestion of IVT enzymes and DNases results in a IVT mixture comprising RNA transcripts, DNA fragments, peptide fragments, individual nucleotides, and individual amino acids. After DNase and protease digestion, the RNA transcripts are larger than the other components of the mixture, and can thus be separated using size-based filtration methods. An example of such a filtration method is tangential flow filtration (TFF). In TFF, a mixture flows over a filtration membrane (TFF membrane) comprising pores, with the pores of the membrane being oriented perpendicular to the direction of flow. Components of the mixture flow through the pores, if able, while components that do not pass through the pores are retained in the mixture. TFF thus removes smaller impurities, such as peptide fragments, DNA fragments, amino acids, and nucleotides from a mixture, while larger molecules, such as full-length RNA transcripts, are retained in the mixture. Additionally, RNA polymerases may produce double-stranded RNA transcripts during IVT, comprising an RNA:RNA hybrid of a full-length RNA transcript and another RNA with a complementary sequence. The second RNA that is hybridized to the full-length RNA transcript may be another full-length RNA, or a smaller RNA that hybridizes to only a portion of the full-length transcript. Like DNA fragments produced by DNase digestion of DNA templates, these small RNAs may also be removed during TFF, so that fewer dsRNA molecules are present in the filtered RNA composition.

The size of the pores of the TFF membrane affect which components are filtered (removed) from the mixture and which are retained in the mixture. Generally, TFF membranes are characterized in terms of a molecular weight cutoff, with components smaller than the molecular weight cutoff being removed from the mixture during TFF, while components larger than the molecular weight cutoff being retained in the mixture. In some embodiments, the tangential flow filtration comprises using a TFF membrane with a molecular weight cutoff of 500 kDa or less, 200 kDa or less, or 100 kDa or less. In some embodiments, the tangential flow filtration comprises using a TFF membrane with a molecular weight cutoff of 500 kDa or less. In some embodiments, the tangential flow filtration comprises using a TFF membrane with a molecular weight cutoff of 400 kDa or less. In some embodiments, the tangential flow filtration comprises using a TFF membrane with a molecular weight cutoff of 300 kDa or less. In some embodiments, the tangential flow filtration comprises using a TFF membrane with a molecular weight cutoff of 200 kDa or less. In some embodiments, the tangential flow filtration comprises using a TFF membrane with a molecular weight cutoff of 100 kDa or less.

### RNase III Digestion

Some aspects of the disclosure relate to methods of reducing the abundance of double-stranded RNA (dsRNA) molecules in a composition comprising mRNA, such as an IVT mixture or an mRNA composition, by introducing an RNase III into the mixture or composition to cleave one or more dsRNA molecules, and isolating mRNA from the mixture or composition. Double-stranded RNA transcripts, in which at least a portion of an RNA transcript is hybridized to another RNA molecule, elicit an innate immune response when introduced into a cell, causing degradation of both strands of a dsRNA. Reducing the abundance of dsRNA molecules enables the production of less immunogenic, and thus more stable, RNA compositions. RNase III enzymes catalyze the cleavage of phosphodiester bonds between nucleotides in a dsRNA. In cells, RNase III enzymes typically produce shorter dsRNA fragments 18-25 base pairs in length, with each RNA strand having two nucleotides at the 3′ terminus that are not bound by complementary nucleotides on the opposing RNA strand. Such shorter dsRNA fragments play a role in RNA-mediated silencing of gene expression, and are known as siRNAs. These smaller dsRNA fragments produced by RNase III digestion are smaller than full-length single-stranded mRNA transcripts encoding proteins, and are thus more easily separated from desired mRNAs.

Ribonuclease III (RNase III) is an endoribonuclease that binds to and cleaves double stranded RNA. The enzyme is expressed in many organisms and is highly conserved (e.g., Mian et al., Nucleic Acids Res., 1997, 25, 3187-95). RNase III species cloned to date contain an RNase III signature sequence and vary in size from 25 to 50 kDa. Multiple functions have been ascribed to RNase III. In both Escherichia coli and Saccharomyces cerevisiae, RNase III is involved in the processing of pre-ribosomal RNA (pre-rRNA) (e.g., Elela et al., Cell, 1996, 85, 115-24). RNase III is also involved in the processing of small molecular weight nuclear RNAs (snRNAs) and small molecular weight nucleolar RNAs (snoRNAs) in S. cerevisiae (e.g., Chanfreau et al., Genes Dev. 1996, 11, 2741-51; Qu et al., Mol. Cell. Biol. 1996, 19, 1144-58). In E. coli, RNase III is involved in the degradation of some mRNA species (e.g., Court et al., Control of messenger RNA stability, 1993, Academic Press, Inc, pp. 71-116).

There are several types of Drosophila and Caenorhabditis elegans RNase III enzymes. The canonical RNase III contains a single RNase III signature motif and a double-stranded RNA binding domain (dsRBD; e.g. RNC_CAEEL). Drosha (Filippov et al. (2000) Gene 245: 213-221) is a Drosophila enzyme that contains two RNase III motifs and a dsRBD (CeDrosha in C. elegans). Another type of RNase III enzyme contains two RNase III signatures and an amino terminal helicase domain (e.g. Drosophila CG4792, CG6493, C. elegans K12H4.8) and may be RNAi nucleases (Bass (2000) Cell 101: 235-238). Enzymes from each Drosophila and Caenorhabditis elegans type produce discrete ˜22 nucleotide (nt) RNAs from dsRNA substrates.

RNase III enzymes that may be used for RNase III digestion include RNase III enzymes that specifically bind to dsRNA. A RNase III enzyme may be a bacterial enzyme or a eukaryotic (e.g., mammalian) enzyme. In some embodiments, a RNase III is an E. coli RNase III (EcR3). In some embodiments, a RNase III is a T. maritima RNase III. In some embodiments, a RNase III is an Aquifex aeolicus RNase III (AaR3). In some embodiments, a RNase III is a human RNase III. In some embodiments, the RNase III is selected from the group consisting of a class 1 RNase III, a class 2 RNase III, a class 3 RNase III, or a class 4 RNase III. Class 1 RNase III enzymes are magnesium-dependent endonucleases that are encoded primarily by bacteria and bacteriophage genomes. Class 2 RNase III enzymes are commonly encoded by fungal genomes, such as yeast of the genus Saccharomyces. Class 3 RNase III enzymes include the Drosha family of enzymes, which are encoded by animal genomes. Class 4 RNase III enzymes include the Dicer family of enzymes, which are encoded by plant genomes. In some embodiments, the RNase III is a class 1 RNase III. In some embodiments, the RNase III enzyme is a class 2 RNase III. In some embodiments, the RNase III enzyme is a class 3 RNase III. In some embodiments, the RNase III enzyme is a class 4 RNase III.

Any RNase III or variant thereof that specifically binds to and cleaves dsRNA may be used for RNase III digestion. Non-limiting examples of RNase III enzymes are listed in Table 1.

TABLE 1
Exemplary RNase III enzyme sequences.

| Host organism | Amino acid sequence |
|---|---|
| Escherichia coli | MNPIVINRLQRKLGYTFNHQELLQQALTHRSASSKHNERLEFLGDSILSYVIANALY |
| (wild-type) | HRFPRVDEGDMSRMRATLVRGNTLAELAREFELGECLRLGPGELKSGGFRRESILAD |
| | TVEALIGGVFLDSDIQTVEKLILNWYQTRLDEISPGDKQKDPKTRLQEYLQGRHLPL |
| | PTYLVVQVRGEAHDQEFTIHCQVSGLSEPVVGIGSSRRKAEQAAAEQALKKLELE |
| | (SEQ ID NO: 4) |
| Escherichia coli | MNPIVINRLQRKLGYTENHQELLQQALTHRSASSKHNARLEFLGDSILSYVIANALY |
| (E38A) | HRFPRVDEGDMSRMRATLVRGNTLAELAREFELGECLRLGPGELKSGGFRRESILAD |
| | TVEALIGGVELDSDIQTVEKLILNWYQTRLDEISPGDKQKDPKTRLQEYLQGRHLPL |
| | PTYLVVQVRGEAHDQEFTIHCQVSGLSEPVVGTGSSRRKAEQAAAEQALKKLELE |
| | (SEQ ID NO: 5) |
| Thermotoga | MNESERKIVEEFQKETGINFKNEELLFRALCHSSYANEQNQAGRKDVESNEKLEFLG |
| maritima (wild- | DAVLELFVCEILYKKYPEAEVGDLARVKSAAASEEVLAMVSRKMNLGKFLFLGKGEE |
| type) | KTGGRDRDSILADAFEALLAAIYLDQGYEKIKELFEQEFEFYIEKIMKGEMLFDYKT |
| | ALQEIVQSEHKVPPEYILVRTEKNDGDRIFVVEVRVNGKTIATGKGRTKKEAEKEAA |
| | RIAYEKLLKERS (SEQ ID NO: 6) |
| Thermotoga | MNESERKIVEEFQKETGINFKNEELLFRALCHSSYANEQNQAGRKDVESNAKLEFLG |
| maritima (E51A) | DAVLELFVCEILYKKYPEAEVGDLARVKSAAASEEVLAMVSRKMNLGKFLFLGKGEE |
| | KTGGRDRDSILADAFEALLAAIYLDQGYEKIKELFEQEFEFYIEKIMKGEMLFDYKT |
| | ALQEIVQSEHKVPPEYILVRTEKNDGDRIFVVEVRVNGKTIATGKGRTKKEAEKEAA |
| | RIAYEKLLKERS (SEQ ID NO: 7) |
| Aquifex aeolicus | MKMLEQLEKKLGYTFKDKSLLEKALTHVSYSKKEHYETLEFLGDALVNFFIVDLLVQ |
| (wild-type) | YSPNKREGFLSPLKAYLISEEFFNLLAQKLELHKFIRIKRGKINETIIGDVFEALWA |
| | AVYIDSGRDANFTRELFYKLFKEDILSAIKEGRVKKDYKTILQEITQKRWKERPEYR |
| | LISVEGPHHKKKFIVEAKIKEYRTLGEGKSKKEAEQRAAEELIKLLEESE |
| | (SEQ ID NO: 8) |
| Aquifex aeolicus | MKMLEQLEKKLGYTFKDKSLLEKALTHVSYSKKEHYATLEFLGDALVNFFIVDLLVQ |
| (E37A) | YSPNKREGFLSPLKAYLISEEFFNLLAQKLELHKFIRIKRGKINETIIGDVFEALWA |
| | AVYIDSGRDANFTRELFYKLFKEDILSAIKEGRVKKDYKTILQEITQKRWKERPEYR |
| | LISVEGPHHKKKFIVEAKIKEYRTLGEGKSKKEAEQRAAEELIKLLEESE |
| | (SEQ ID NO: 9) |

In some embodiments, the RNase III is an Escherichia coli RNase III. In some embodiments, the RNase III comprises an amino acid sequence with at least 80%, at least 85%, at least 90%, at least 95%, at least 97%, at least 98%, or at least 99% sequence identity to the amino acid sequence of SEQ ID NO: 4. In some embodiments, the RNase III comprises the amino acid sequence of SEQ ID NO: 4. In some embodiments, the RNase III comprises an amino acid sequence with at least 80%, at least 85%, at least 90%, at least 95%, at least 97%, at least 98%, or at least 99% sequence identity to the amino acid sequence of SEQ ID NO: 4, and comprises an amino acid substitution corresponding to an E38A substitution in SEQ ID NO: 4. In some embodiments, the RNase III comprises the amino acid sequence of SEQ ID NO: 5.

In some embodiments, the RNase III is a Thermotoga maritima RNase III. In some embodiments, the RNase III comprises an amino acid sequence with at least 80%, at least 85%, at least 90%, at least 95%, at least 97%, at least 98%, or at least 99% sequence identity to the amino acid sequence of SEQ ID NO: 6. In some embodiments, the RNase III comprises the amino acid sequence of SEQ ID NO: 6. In some embodiments, the RNase III comprises an amino acid sequence with at least 80%, at least 85%, at least 90%, at least 95%, at least 97%, at least 98%, or at least 99% sequence identity to the amino acid sequence of SEQ ID NO: 6, and comprises an amino acid substitution corresponding to an E38A substitution in SEQ ID NO: 6. In some embodiments, the RNase III comprises the amino acid sequence of SEQ ID NO: 7.

In some embodiments, the RNase III is an Aquifex aeolicus RNase III. In some embodiments, the RNase III comprises an amino acid sequence with at least 80%, at least 85%, at least 90%, at least 95%, at least 97%, at least 98%, or at least 99% sequence identity to the amino acid sequence of SEQ ID NO: 8. In some embodiments, the RNase III comprises the amino acid sequence of SEQ ID NO: 8. In some embodiments, the RNase III comprises an amino acid sequence with at least 80%, at least 85%, at least 90%, at least 95%, at least 97%, at least 98%, or at least 99% sequence identity to the amino acid sequence of SEQ ID NO: 8, and comprises an amino acid substitution corresponding to an E38A substitution in SEQ ID NO: 8. In some embodiments, the RNase III comprises the amino acid sequence of SEQ ID NO: 9.

“Sequence identity” herein refers to the overall relatedness among polypeptides, for example, among RNase III and variants thereof. The percent sequence identity of two polypeptide sequences, for example, can be calculated by aligning the two sequences for optimal comparison purposes (e.g., gaps can be introduced in one or both of a first and a second nucleic acid sequences for optimal alignment and non-identical sequences can be disregarded for comparison purposes). In some embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two amino acid sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

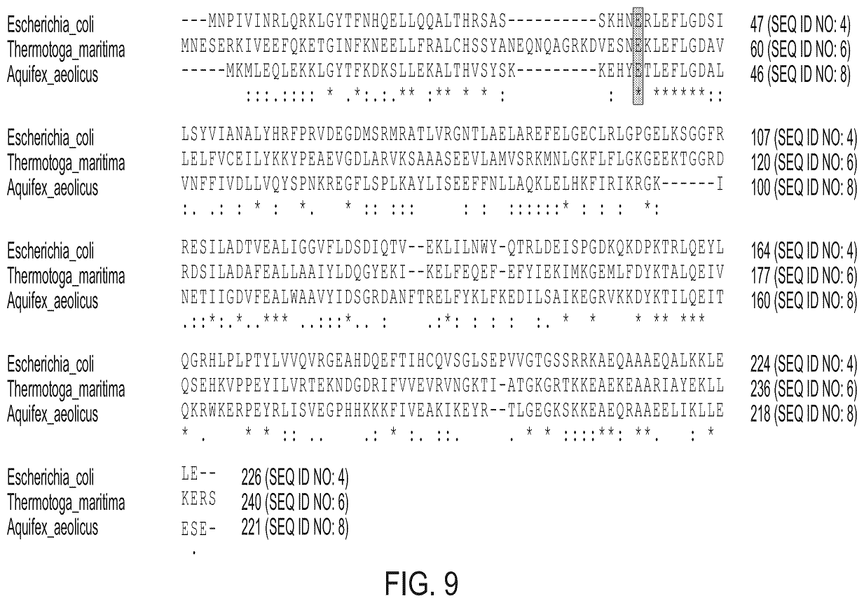

The term “corresponding to” an amino acid position in a sequence means that when two sequences (e.g., polypeptide sequence) are aligned (e.g., using any of the known sequence alignment programs in the art such as the ones described herein), a certain amino acid residue in one sequence aligns with an amino acid residue in the other sequence, these two amino acid residues are considered to be “corresponding to” each other. Thus, two amino acid residues that correspond to each other may not necessarily have the same numerical position. For example, a glutamic acid (E) residue that, when substituted for an alanine (A), reduces the off-target degradation of single-stranded RNA by RNase III, is located at position 38 of the amino acid identified by SEQ ID NO: 4 (E. coli). The corresponding glutamic acid (E) is located at position 51 of the amino acid identified by SEQ ID NO: 6 (T. maritima), and the corresponding glutamic acid (E) is located at position 37 of the amino acid identified by SEQ ID NO: 8 (A. aeolicus) (FIG. 9).

In some embodiments, after the RNase III is introduced into the IVT mixture or RNA composition, the concentration of the RNase III in the mixture or composition is about 0.01 U/mL to about 0.5 U/mL. In some embodiments, a “Unit” of RNase III refers to the amount of enzyme required to digest 1 μg of dsRNA in 50 μL reaction volume. In some embodiments, a “Unit” of RNase III refers to the amount of enzyme required to digest 1 μg of dsRNAs that are 500 bp in length to produce fragments between 12-30 bp in length. In some embodiments, the concentration of RNase III in the mixture is 0.5 U/mL or lower, 0.4 U/mL or lower, 0.3 U/mL or lower, 0.25 U/mL or lower, 0.2 U/mL or lower, 0.15 U/mL or lower, 0.1 U/mL or lower, 0.09 U/mL or lower, 0.08 U/mL or lower, 0.07 U/mL or lower, 0.06 U/mL or lower, 0.05 U/mL or lower, 0.04 U/mL or lower, 0.03 U/mL or lower, 0.02 U/mL or lower, or 0.01 U/mL or lower.

In some embodiments, the step of RNase III digestion is conducted by incubating the RNase III at about 37° C. In some embodiments, the RNase III digestion step is conducted at a temperature of 70° C. or lower, 60° C. or lower, 50° C. or lower, 40° C. or lower, 30° C. or lower, or 25° C. or lower. In some embodiments, the step of RNase III digestion is conducted for about 10 minutes to about 6 hours. In some embodiments, the step of RNase III digestion is conducted for at least 10 minutes, at least 15 minutes, at least 20 minutes, at least 30 minutes, at least 40 minutes, at least 50 minutes, or at least 60 minutes. In some embodiments, the step of RNase III digestion is conducted for at least 1 hour, at least 2 hours, at least 3 hours, at least 4 hours, or at least 5 hours. In some embodiments, the step of RNase III digestion is conducted for about 10 minutes. In some embodiments, the step of RNase III digestion is conducted for about 15 minutes. In some embodiments, the step of RNase III digestion is conducted for about 20 minutes. In some embodiments, the step of RNase III digestion is conducted for about 30 minutes. In some embodiments, the step of RNase III digestion is conducted for about 45 minutes. In some embodiments, the step of RNase III digestion is conducted for about 60 minutes. In some embodiments, the step of RNase III digestion is conducted for a period of time sufficient to cleave one or more dsRNAs in the composition or mixture. In some embodiments, the step of RNase III digestion is conducted for a period of time sufficient to cleave at least 80%, at least 85%, at least 90%, at least 95%, at least 97%, at least 98%, at least 99%, or up to 100% of dsRNAs in the mixture. A “period of time sufficient” to achieve an outcome refers to a length of time which, if allowed to pass, causes the outcome to be achieved. The period of time sufficient to cleave one or more dsRNAs, or to cleave a certain percentage of dsRNAs in a mixture, may be determined by incubating RNase III in a composition comprising dsRNA, sampling the composition after the passage of multiple periods of time, and determining the extent of dsRNA cleavage at each sampling time. If a given outcome has been achieved after the passage of a given period of time, that period of time is said to be sufficient to achieve the outcome. A period of time sufficient to cleave a certain percentage of dsRNAs refers to the period of time, after which at least that percentage of dsRNAs that were initially present at the start of the incubation have been cleaved by the RNase III. Thus, after a period of time sufficient to cleave 80% of dsRNAs in a mixture, at least 80% of dsRNAs that were initially present will have been cleaved by RNase III.

In some embodiments, the IVT mixture or RNA composition comprises magnesium ions during the step of incubating the RNase III. The presence and concentrations of different cations can affect the activity of an RNase III. For example, some RNase III enzymes are more active in the presence of divalent cations, such as magnesium (Mg²⁺). The cations present during RNase III digestion also affect the specificity of the enzyme. For example, certain RNAse III enzymes are more specific in the presence of magnesium ions compared to an equivalent concentration of other cations, such as manganese (Mn²⁺) ions. In some embodiments, the cation present in or added to the mixture or composition is a magnesium ion. In some embodiments, the concentration of magnesium ions in the mixture or composition during RNase III digestion is about 10 mM to about 100 mM. In some embodiments, the concentration of magnesium ions is about 1 mM to about 5 mM, about 5 mM to about 10 mM, about 10 mM to about 20 mM, about 20 mM to about 30 mM, about 30 mM to about 40 mM, about 40 mM to about 50 mM, about 50 mM to about 60 mM, about 60 mM to about 70 mM, about 70 mM to about 80 mM, about 80 mM to about 90 mM, or about 90 to about 100 mM. In some embodiments, the concentration of magnesium ions is about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, or about 30 mM. In some embodiments, the concentration of magnesium ions is about 10 mM. In some embodiments, the concentration of magnesium ions is about 20 mM. In some embodiments, the concentration of magnesium ions is about 30 mM.

In some embodiments, the method comprises filtering a mixture or composition after incubating the RNase III, to separate smaller dsRNA or single-stranded RNA fragments produced by RNase III from desired mRNA transcripts. In some embodiments, the filtration is conducted by tangential flow filtration. In some embodiments, the tangential flow filtration comprises using a TFF membrane with a molecular weight cutoff of 500 kDa or less, 200 kDa or less, or 100 kDa or less. In some embodiments, the tangential flow filtration comprises using a TFF membrane with a molecular weight cutoff of 500 kDa or less. In some embodiments, the tangential flow filtration comprises using a TFF membrane with a molecular weight cutoff of 400 kDa or less. In some embodiments, the tangential flow filtration comprises using a TFF membrane with a molecular weight cutoff of 300 kDa or less. In some embodiments, the tangential flow filtration comprises using a TFF membrane with a molecular weight cutoff of 200 kDa or less. In some embodiments, the tangential flow filtration comprises using a TFF membrane with a molecular weight cutoff of 100 kDa or less.

In some embodiments, after isolating mRNA from the mixture or composition after RNase III digestion, the concentration of dsRNA in the isolated mRNA composition is 5% (% w/w) or less, 4% or less, 3% or less, 2.5% or less, 2% or less, 1.75% or less, 1.5% or less, 1.25% or less, 1% or less, 0.9% or less, 0.8% or less, 0.7% or less, 0.6% or less, 0.5% or less, 0.4% or less, 0.3% or less, 0.25% or less, 0.2% or less, 0.175% or less, 0.15% or less, 0.125% or less, or 0.1% or less. In some embodiments, the concentration of double-stranded RNA in a composition comprising RNA is 0.05% (% w/w) or less, 0.04% or less, 0.03% or less, 0.02% or less, or 0.01% or less. Methods of measuring the presence and/or amount of dsRNA in a composition are known in the art. Non-limiting examples of methods for measuring dsRNA content of a sample include ELISAs and immunoblotting using antibodies specific to dsRNA. Additionally, the total mass of RNA in a sample can be measured using techniques such as spectroscopy (NanoDrop), qRT-PCR, and/or ddPCR, and the mass of dsRNA can be measured using an intercalating agent that fluoresces when bound to dsRNA, such as acridine orange, with the dsRNA concentration being calculated by division. In some embodiments, the concentration of dsRNA in a composition refers to the mass of RNA nucleotides that are part of a double-stranded RNA:RNA hybrid, with other unhybridized nucleotides from either RNA in the hybrid not contributing to the amount of dsRNA in a composition. In some embodiments, the concentration of dsRNA in a composition refers to the concentration of RNA molecules containing nucleotides that are part of an RNA:RNA hybrid. In some embodiments, the concentration of dsRNA in a composition refers to the proportion of RNA polynucleotides molecules that are part of an RNA:RNA hybrid.

### Salt Precipitation

Some aspects of the disclosure relate to methods of introducing a salt into an IVT mixture after RNA has been transcribed to precipitate the RNA. After the RNA is precipitated, proteins and other components of the IVT mixture can be removed by filtering the solution and/or washing the precipitated RNA. After the precipitated RNA is washed and/or filtered to remove proteins, the RNA can be resolubilized and dissolved in a protein-free solvent to produce an RNA composition. The sugar-phosphate backbone of nucleic acids, such as RNA, includes negatively charged phosphate ions, which makes individual RNA molecules hydrophilic and able to be dissolved in water. Adding a salt containing positive ions, such as sodium or lithium ions, neutralizes the negative charge of these phosphate groups, making RNA molecules less hydrophilic. After adding the salt, the addition of a less polar solution, such as ethanol, allows the cations to more easily neutralize the negative charge of the RNA phosphates, resulting in RNA precipitating, while other components of the solution remain dissolved.

In some embodiments, the salt is selected from the group consisting of lithium chloride, sodium chloride, sodium acetate, ammonium acetate, calcium chloride, and ammonium sulfate. In some embodiments, the salt is lithium chloride or sodium acetate. In some embodiments, the salt is lithium chloride. In some embodiments, the salt is sodium acetate. The use of lithium chloride in salt precipitation results in the precipitation of RNA, but not DNA or proteins, which allows for the removal of DNA templates as well as IVT and other enzymes from the IVT mixture, to produce a more pure RNA composition. In some embodiments, the concentration of salt in the mixture after addition is 0.1 M to about 10 M, about 0.2 M to about 5 M, or 0.5 M to about 2.5 M. In some embodiments, the concentration of salt is about 0.1 M to about 1 M, about 1 M to about 2 M, about 2 M to about 3 M, about 3 M to about 4 M, or about 4 M to about 5 M. In some embodiments, the concentration of salt is about 1 M. In some embodiments, the concentration of salt is about 2 M. In some embodiments, the concentration of salt is about 3 M. In some embodiments, the concentration of salt is about 4 M. In some embodiments, the concentration of salt is about 5 M.

In some embodiments, after the RNA is precipitated, proteins, DNA templates, and other dissolved components of an IVT mixture are separated from the RNA by washing the precipitated RNA with one or more washing solutions. A washing solution refers to a solution in which precipitated RNA is minimal. Generally, a washing solution contains a salt that is capable of precipitating RNA and/or an alcohol. Aspiration of the supernatant from a precipitated RNA pellet removes many dissolved proteins and DNAs, and the addition of a washing solution further dilutes any residual proteins and DNAs present in the solution. Repeated steps of supernatant aspiration and washing remove soluble proteins and DNAs, thereby increasing the purity of the RNA. In some embodiments, the washing solution comprises a salt selected from the group consisting of lithium chloride, sodium chloride, sodium acetate, ammonium acetate, calcium chloride, and ammonium sulfate. In some embodiments, the washing solution comprises lithium chloride. In some embodiments, the concentration of salt in the washing solution is 0.1 M to about 10 M, about 0.2 M to about 5 M, or 0.5 M to about 2.5 M. In some embodiments, the concentration of salt is about 0.1 M to about 1 M, about 1 M to about 2 M, about 2 M to about 3 M, about 3 M to about 4 M, or about 4 M to about 5 M. In some embodiments, the concentration of salt is about 1 M. In some embodiments, the concentration of salt is about 2 M. In some embodiments, the concentration of salt is about 3 M. In some embodiments, the concentration of salt is about 4 M. In some embodiments, the concentration of salt is about 5 M. In some embodiments, the washing solution comprises ethanol. In some embodiments, the washing solution comprises isopropanol. In some embodiments, the washing solution comprises at least 60% (% w/v), at least 65%, at least 70%, at least 75%, at least 80%, at least 85%, at least 90%, at least 95%, or up to 100% alcohol. In some embodiments, the washing solution comprises about 70% alcohol.

In some embodiments, after the RNA is precipitated, the composition containing the precipitated RNA is filtered to separate the solution containing salt, protein, and DNA from the precipitated RNA. In some embodiments, the step of filtering comprises adding the precipitated RNA and supernatant to a hollow fiber filter. A hollow fiber filter is a membrane comprising a network of fibers, and pores formed by the networked fibers. In contrast to tangential flow, in which a liquid or composition flows parallel to a TFF membrane, the supernatant and precipitated RNA are applied to the hollow fiber filter such that the direction of liquid flow is through the filter (axial flow). Flow of the liquid may occur due to gravity, or be aided through the use of negative pressure below the filter, to facilitate filtration. In some embodiments, a vacuum is applied to facilitate the flow of liquid through the filter. Liquids, dissolved solutes, and suspended particles that are smaller than the pores of the filter pass through the pores, while solid components larger than the pores are retained above the filter. Thus, the precipitated RNA is retained above the filter, while the supernatant passes through the filter. Following passage of the supernatant through the filter, a washing solution may be added to the precipitated RNA to further remove any residual proteins or DNAs from the RNA. In some embodiments, the hollow fiber filter has as a pore size of 10 μm or less, 9 μm or less, 8 μm or less, 7 μm or less, 6 μm or less, 5 μm or less, 4 μm or less, 3 μm or less, 2 μm or less, 1 μm or less, 0.9 μm or less, 0.8 μm or less, 0.7 μm or less, 0.6 μm or less, 0.5 μm or less, 0.4 μm or less, 0.3 μm or less, or 0.2 μm or less. In some embodiments, the hollow fiber filter has as a pore size of 30 μm or less. In some embodiments, the hollow fiber filter has as a pore size of 20 μm or less. In some embodiments, the hollow fiber filter has as a pore size of 10 μm or less. In some embodiments, the hollow fiber filter has as a pore size of 5 μm or less. In some embodiments, the hollow fiber filter has as a pore size of 2 μm or less. In some embodiments, the hollow fiber filter has as a pore size of 1 μm or less. In some embodiments, the hollow fiber filter has as a pore size of 0.5 μm or less.

In some embodiments, the step of filtering comprises tangential flow filtration. In some embodiments, the tangential flow filtration comprises using a TFF membrane with a molecular weight cutoff of 500 kDa or less, 200 kDa or less, or 100 kDa or less. In some embodiments, the tangential flow filtration comprises using a TFF membrane with a molecular weight cutoff of 500 kDa or less. In some embodiments, the tangential flow filtration comprises using a TFF membrane with a molecular weight cutoff of 400 kDa or less. In some embodiments, the tangential flow filtration comprises using a TFF membrane with a molecular weight cutoff of 300 kDa or less. In some embodiments, the tangential flow filtration comprises using a TFF membrane with a molecular weight cutoff of 200 kDa or less. In some embodiments, the tangential flow filtration comprises using a TFF membrane with a molecular weight cutoff of 100 kDa or less.

After one or more washing and/or filtering steps, the precipitated RNA can be resolubilized. Resolubilization refers to the removal of positive ions, such lithium ions of the salt used in precipitation or washing solution. In the absence of such ions, the phosphates of the precipitated RNA more readily interact with water molecules, allowing the RNA to be dissolved in another solution. In some embodiments, resolubilizing step comprises washing the precipitated RNA with a resolubilizing solution, then resuspending the RNA in a resuspension solution. A resolubilizing solution is a solution that is less conductive than a washing solution, and in which precipitated RNA is minimally soluble. Successive steps of adding a resolubilizing solution to the precipitated RNA, followed by aspirating the supernatant from the precipitated RNA, remove ions of the precipitating salt and/or washing solution. In some embodiments, the precipitated RNA is washed with resolubilizing solution more than once. In some embodiments, the resolubilizing solution comprises citrate. Citrate solutions are generally less conductive than other salt solutions such as lithium chloride or sodium acetate solutions. Furthermore, the presence of citrate in a composition comprising RNA can reduce the frequency of base hydrolysis, thereby maintaining the stability of full-length RNA transcripts in the precipitated RNA during resolubilization.

After ions are removed from the precipitated RNA, the conductivity of a liquid, such as a resolubilizing solution or resuspension solution, that is added to precipitated RNA will be lower. In some embodiments, the resolubilizing solution, after addition to precipitated RNA, has a conductivity of 50 mS/cm or less, 40 mS/cm or less, 30 mS/cm or less, 20 mS/cm or less, or 10 mS/cm or less. At low conductivities, the precipitated RNA can be redissolved by adding a resuspension solution. In some embodiments, the resuspension solution comprises a Tris buffer. Tris refers to tris(hydroxymethyl)aminomethane, is an organic compound commonly used in buffers for resuspension and storage of nucleic acids such as DNA and RNA.

In some embodiments, the steps of salt precipitating the RNA, filtering the precipitated RNA, and resolubilizing the RNA are conducted before a protease is introduced into the IVT mixture or to the RNA. In some embodiments, the steps of salt precipitating the RNA, filtering the precipitated RNA, and resolubilizing the RNA are conducted after a protease is removed from the IVT mixture or RNA by filtration.

Some aspects of the disclosure relate to methods for purifying in vitro transcribed mRNA, wherein the method comprises contacting a mixture comprising the mRNA with a salt in an amount sufficient to precipitate the mRNA, separating the precipitated mRNA from one or more proteins in the mixture, and resolubilizing the mRNA.

Removal of mRNA During Continuous In Vitro Transcription

Some aspects of the disclosure relate to continuous in vitro transcription methods in which, during the transcription process, a portion of the IVT mixture is removed from the reaction vessel and filtered through the stationary phase of a column. In some aspects, the disclosure relates to methods of purifying in vitro transcribed mRNA, wherein the mRNA is produced by IVT methods that include removing mRNA from the reaction vessel. Filtering the portion of the reaction mixture through the stationary phase of the column allows RNA transcripts to be retained in the stationary phase of the column, while other components of the IVT mixture, such as RNA polymerases, capping enzymes, polyadenylating enzymes, DNA templates, and nucleotide triphosphates (NTPs), can be eluted from the column and reintroduced into the reaction vessel. Removing RNA from the reaction vessel in this manner reduces the concentration of RNA in the IVT reaction, which is useful for preventing the self-inhibitory effects of RNA on in vitro transcription. Unlike recombinant protein production, where the cells that produce and secrete recombinant proteins are significantly larger than the secreted proteins, enabling proteins and cells to be separated by size exclusion chromatography, mRNAs produced by IVT are similar, in both size and structure, to other components of the IVT mixture, such as the DNA templates encoding the mRNA. The structural similarity between the mRNA products and the DNA templates to be reintroduced back into the IVT mixture reduces the feasibility of conventional approaches, such as size exclusion chromatography, for separating produced mRNA from other IVT components. Alternative approaches are thus required to separate produced mRNA from other components of an IVT mixture without also removing important components such as DNA template molecules. Furthermore, IVT requires multiple components, such as a DNA template, RNA polymerase, and cofactor ions, NTPs, to produce an mRNA. The removal of any one of these components may interfere with the production of mRNA, and so methods of separating mRNA from an IVT reaction must maintain a sufficient concentration of other IVT reagents (e.g., DNA templates, RNA polymerases, cofactor ions, and 5′ caps) in the IVT mixture to enable continued IVT.

In some embodiments, affinity capture of mRNA is used to separate produced mRNA from other components of an IVT mixture. “Affinity capture” refers to a process of isolating a component of a mixture (e.g., mRNA in an IVT reaction mixture) based on its increased affinity, relative to other components of the mixture, for a reagent used to capture the component. For example, an IVT mixture containing produced mRNA may be contacted with a stationary phase having a higher affinity for mRNA than other components of the IVT mixture, causing mRNA to be bound by the stationary phase, resulting in the IVT mixture having a lower concentration of mRNA after contacting the stationary phase. In some embodiments, the stationary phase comprises oligo-dT. Oligo-dT refers to an oligonucleotide comprising consecutive thymidine deoxyribonucleotides (dT), which hybridize with the poly(A) tail of mRNA, allowing oligo-dT-containing stationary phase to retain mRNAs. In some embodiments, the stationary phase comprises fiber, particles, resin, and/or beads. In some embodiments, the stationary phase comprises oligo-dT fiber. Unlike resin or bead-based chromatography stationary phases, oligo-dT fibers comprise a porous matrix of cellulose fibers comprising oligo-dT on their surface. Solutions containing mRNA, such as samples from an IVT reaction mixture, may be passed through the porous cellulose fiber matrix. One benefit of using oligo-dT fiber to remove mRNA from an IVT mixture is that a liquid IVT mixture can quickly contact many oligo-dT molecules of the fiber, allowing efficient mRNA capture and reintroduction of the IVT mixture back into a larger reaction vessel. Capturing many mRNAs in a short amount of time allows efficient removal of mRNA from the IVT mixture, minimizing the time before the IVT mixture is reintroduced to the reaction vessel, and thus increasing the amount of time other components of the IVT mixture are active in mRNA production. Increasing the efficiency of mRNA capture, and consequently the utilization of IVT components in mRNA production, thus increases the productivity of IVT, in terms of the amount of mRNA produced using a given amount of IVT components in a given length of time.

In some embodiments, the method further comprises eluting RNA from the column to obtain an eluate comprising RNA. In some embodiments, the eluting step is performed more than once. In some embodiments, the steps of adding a portion of the IVT mixture to stationary phase of the column, retaining RNA in the column, and re-introducing the flowthrough from the column into the IVT reaction vessel are repeated after the eluting step. In some embodiments, the elution step is performed after the amount of mRNA bound to the stationary phase reaches a defined threshold and/or after the concentration of mRNA in the IVT mixture has been reduced by at least a defined amount. Whether the amount of mRNA bound to a stationary phase has reached a defined threshold may be determined by quantifying the amount of mRNA associated with the stationary phase, and/or by quantifying the amount of mRNA in the IVT mixture before and after contact with the stationary phase and accordingly calculating the amount of mRNA removed by contact with the stationary phase. In some embodiments, elution is performed after the amount of mRNA bound to the stationary phase is at least 80%, 85%, 90%, 95%, 96%, 97%, 98%, 99%, or up to 100% of the mRNA-binding capacity of the stationary phase. In some embodiments, elution is performed after an amount of mRNA corresponding to at least 80%, 85%, 90%, 95%, 96%, 97%, 98%, 99%, 100%, or more of the mRNA-binding capacity of the stationary phase is removed from the IVT mixture. mRNA-binding capacity of a stationary phase may be evaluated by one of any methods known in the art. For example, binding capacity may be determined by contacting a stationary phase with a solution containing mRNA, measuring the concentration of mRNA in the solution at multiple timepoints, and calculating the total loss of mRNA in solution, corresponding to mRNA bound to the stationary phase, from the asymptotic limit of mRNA concentration. Alternatively, a stationary phase may be contacted with a solution containing a concentration of mRNA for a period of time sufficient to allow saturation of the stationary phase, after which an elution buffer may be used to remove bound mRNA from the stationary phase, with the amount of eluted mRNA indicating the binding capacity of the stationary phase.

In some embodiments, elution is performed after the stationary phase has been contacted with a solution containing mRNA for a defined length of time, such as a length of time that is expected to saturate the stationary phase or cause an amount of mRNA corresponding to a defined percentage of the mRNA-binding capacity of the stationary phase. A defined length of time may be determined based on the binding capacity of the stationary phase and/or the concentration of mRNA in an IVT reaction mixture that is contacted with the stationary phase. For example, a solution containing a known starting concentration of mRNA may be exposed to a stationary phase, with the concentration of mRNA in solution being measured over time to determine the time after which removal of mRNA from the solution, corresponding to binding by the stationary phase, slows or stops. When a continuous IVT process is conducted such that the mRNA concentration is maintained at or near a similar concentration, contacting the stationary phase with the IVT reaction mixture for a similar length of time can similarly saturate, or bind a similar amount of mRNA, to the stationary phase. In some embodiments, elution is performed after 2, 3, 4, 5, 6, 7, 8, 9, 10, 11, 12, 13, 14, 15, 16, 17, 18, 19, 20, 21, 22, 23, 24, 25, 26, 27, 28, 29, or 30 minutes of contacting the stationary phase with the IVT reaction mixture containing mRNA. In some embodiments, elution is performed after 2-5, 5-8, 8-12, 12-15, 15-20, 20-25, 25-30, 30-40, 40-50, or 50-60 minutes of contacting the stationary phase with the IVT reaction mixture containing mRNA. In some embodiments, elution is performed after 1-60, 1-30, 1-20, 1-15, 1-10, 1-5, 2-60, 2-30, 2-20, 2-15, 2-10, 2-5, 5-45, 5-30, 5-15, 5-10, 5-9, 5-8, 5-7, or 5-6 minutes of contacting the stationary phase with the IVT reaction mixture containing mRNA. In some embodiments, elution is performed after 3 minutes. In some embodiments, elution is performed after 4 minutes. In some embodiments, elution is performed after 5 minutes. In some embodiments, elution is performed after 6 minutes. In some embodiments, elution is performed after 7 minutes. In some embodiments, elution is performed after 8 minutes. In some embodiments, elution is performed after 9 minutes. In some embodiments, elution is performed after 10 minutes. Repeating these steps of filtering a portion of the IVT mixture through a column, re-introducing the flowthrough to the IVT reaction vessel, and eluting RNA from the column into a separate collection vessel, allows transcribed RNA to be continuously separated from the IVT reaction. Removing transcribed RNA in this manner maintains the concentration of RNA in the reaction below a desired threshold, thereby preventing the inhibitory effects of transcribed RNA on further transcription. Continuous maintenance of RNA concentration below such a threshold thus maintains the rate of mRNA production, allowing a greater amount of RNA to be produced from a given amount of starting material in a given length of time. In some embodiments, the concentration of RNA in the reaction vessel is maintained at a concentration of 20 mg/mL or less, 15 mg/mL or less, 12 mg/mL or less, or 10 mg/mL or less. In some embodiments, the concentration of RNA is maintained at a concentration of 20 mg/mL or less. In some embodiments, the concentration of RNA is maintained at a concentration of 15 mg/mL or less. In some embodiments, the concentration of RNA is maintained at a concentration of 10 mg/mL or less. In some embodiments, the concentration of RNA is maintained at a concentration of 5 mg/mL or less.

Continuous maintenance of mRNA concentration in an IVT reaction also allows efficient harvesting of produced mRNA, due to the kinetics of mRNA capture by the stationary phase. IVT reaction mixtures with higher mRNA concentrations saturate the stationary phase, or result in its binding an amount of mRNA corresponding to a defined percentage of the binding capacity, in a shorter amount of time than solutions with lower mRNA concentrations. Thus, in some embodiments, the concentration of RNA in the reaction vessel is maintained at a concentration of 5 mg/mL or more, 6 mg/mL or more, 7 mg/mL or more, 8 mg/mL or more, 9 mg/mL or more, 10 mg/mL or more, 11 mg/mL or more, 12 mg/mL or more, 13 mg/mL or more, 14 mg/mL or more, or 15 mg/mL or more. In some embodiments, the concentration of RNA in the reaction vessel is maintained at a concentration between 5-20 mg/mL, 5-15 mg/mL, 5-10 mg/mL, 8-20 mg/mL, 8-15 mg/mL, 8-12 mg/mL, 8-10 mg/mL, 10-20 mg/mL, 10-15 mg/mL, 10-12 mg/mL, 12-20 mg/mL, or 12-15 mg/mL.

Maintenance of mRNA at or above a desired concentration is achieved, in some embodiments, by maintaining the concentration of IVT reaction components (e.g., DNA templates, RNA polymerases, NTPs) at or near concentrations sufficient to maintain a sufficient rate of transcription, and thus mRNA production. Concentrations of each component to be maintained during IVT may be determined by any method known in the art. For example, the concentration of a component may be varied systematically in parallel reaction vessels, with the rate of transcription or mRNA production being measured in each vessel, to determine the concentration below which transcription becomes limiting. The concentration of each component may be varied independently, or concentrations of multiple components may be varied systematically in combination, to determine the suitability of combinations of IVT component concentrations for continuous IVT. After determining suitable concentrations of reaction components, continuous IVT may be initiated in a reaction vessel containing any suitable concentration of each component, or suitable combination of concentration components.

In producing mRNA, IVT consumes nucleotide triphosphates as they are incorporated into transcribed RNAs, reducing the NTP concentrations in the reaction vessel. Furthermore, while DNA templates and RNA polymerases are not consumed by transcription, degradation of each may still occur, reducing the efficiency of IVT. Additionally, during circulation of the IVT mixture over a stationary phase, some DNA templates and RNA polymerases present in IVT reaction mixtures may not be reintroduced back into the IVT reaction vessel, reducing the concentration of DNA templates and RNA polymerase in the reaction vessel. To counteract the potential reductions in efficiency due to NTP consumption, and degradation or loss of other components during mRNA capture and recirculation, IVT components may be introduced into the reaction vessel during IVT, such that the concentration of one or more NTPs, DNA templates, and RNA polymerases are maintained at or near a desired concentration, or within a desired range of concentrations. In some embodiments, the concentration of a component is maintained by adding the component to the IVT reaction vessel at similar rate to the rate at which the component is consumed by IVT. In some embodiments, the component is added at a rate that is within 80% to 120%, 90% to 110%, 95% to 105%, 97% to 103%, 98% to 102%, or 99% to 101% of the rate at which the component is consumed by IVT.

In some embodiments, the component is added by one or more boluses during the IVT reaction. Bolus addition refers to the discrete, rather than continuous, addition of a component to a mixture. Bolus additions of components avoid the need to maintain continuous rates of component addition. In some embodiments, a bolus addition is made before the concentration of an IVT reaction component falls below a defined level. In some embodiments, a bolus addition of an IVT component increases the concentration of the component to a level that does not exceed a defined level. In some embodiments, a series of bolus additions of IVT reaction components maintains one or more reaction component concentrations within a desired range. Desired concentrations, or concentration ranges, may be determined as described above by measuring the effects of each component's concentration on the efficiency of IVT.

The composition of NTPs in an IVT reaction may also vary. In some embodiments, each NTP in an IVT reaction is present in an equimolar amount. In some embodiments, each NTP in an IVT reaction is present in non-equimolar amounts. For example, ATP may be used in excess of GTP, CTP, and UTP. As a non-limiting example, an IVT reaction may include 7.5 millimolar GTP, 7.5 millimolar CTP, 7.5 millimolar UTP, and 3.75 millimolar ATP. In some embodiments, the molar ratio of G:C:U:A is 2:1:0.5:1. In some embodiments, the molar ratio of G:C:U:A is 1:1:0.7:1. In some embodiments, the molar ratio of G:C:A:U is 1:1:1:1. In some embodiments, the amount of NTPs in an IVT reaction is calculated empirically. For example, the rate of consumption for each NTP in an IVT reaction may be empirically determined for a given input DNA, and then balanced ratios of NTPs based on those individual NTP consumption rates may be provided in the initial composition of an IVT reaction mixture and/or a feed solution used to maintain the concentration and ratios of NTPs in the IVT reaction vessel. International Patent Application No. PCT/US2017/051674 (Publ. No. WO 2018/053209) provides a listing of ratios of nucleotide triphosphates, and optionally nucleotide diphosphates, that may be utilized in continuous in vitro transcription methods. This publication is incorporated by reference herein for this purpose.

In some embodiments, the initial concentration of GTP in the IVT reaction vessel is at least 2 times that of any one or more of ATP, CTP, and UTP. Some RNA polymerases, such as T7 RNA polymerase, initiate transcription at a transcription start site that begins with two guanosine nucleotides, and so including an excess of GTP in an IVT reaction mixture allows efficient transcription initiation. In some embodiments, the initial ratio of GTP:ATP in the IVT reaction vessel is 2-50, 2-40, 2-30, 2-20, 2-10, 2-5, 2-4, 4-50, 4-40, 4-30, 4-20, 4-10, or 4-5. In some embodiments, the initial ratio of GTP:CTP in the IVT reaction vessel is 2-50, 2-40, 2-30, 2-20, 2-10, 2-5, 2-4, 4-50, 4-40, 4-30, 4-20, 4-10, or 4-5. In some embodiments, the initial ratio of GTP:UTP in the IVT reaction vessel is 2-50, 2-40, 2-30, 2-20, 2-10, 2-5, 2-4, 4-50, 4-40, 4-30, 4-20, 4-10, or 4-5. In some embodiments, the initial ratio of GTP:ATP is between about 2× to about 4×. In some embodiments, the initial ratio of GTP:CTP is between about 2× to about 4×. In some embodiments, the initial ratio of GTP:UTP is between about 2× to about 4×. In some embodiments, the initial ratio of GTP:ATP is about 2×. In some embodiments, the initial ratio of GTP:CTP is about 2×. In some embodiments, the initial ratio of GTP:ATP is about 4×. In some embodiments, the initial ratio of GTP:ATP:CTP:UTP at the start of continuous IVT is 4:2:1:1. In some embodiments, the ratio of GTP:ATP:CTP:UTP at the start of continuous IVT is 4:2:2:1. In some embodiments, the ratio of GTP:ATP:CTP:UTP at the start of continuous IVT is 6:3:3:1.

In some embodiments, the IVT reaction mixture further comprises guanosine diphosphate (GDP). GDP may be incorporated into transcribed RNA in place of GTP, forming the same guanosine nucleotide in the transcribed RNA that would be formed by incorporation of GTP. Thus, GDP may be present in combination with GTP such that the cumulative concentration of GTP plus GDP is in excess of one or more other NTPs, to provide similar benefits to transcription efficiency as using GTP in excess of one or more nucleotides.

In some embodiments, the initial ratio of GTP plus GDP to ATP in the IVT reaction vessel is 2-50, 2-40, 2-30, 2-20, 2-10, 2-5, 2-4, 4-50, 4-40, 4-30, 4-20, 4-10, or 4-5. In some embodiments, the initial ratio of GTP plus GDP to CTP in the IVT reaction vessel is 2-50, 2-40, 2-30, 2-20, 2-10, 2-5, 2-4, 4-50, 4-40, 4-30, 4-20, 4-10, or 4-5. In some embodiments, the initial ratio of GTP plus GDP to UTP in the IVT reaction vessel is 2-50, 2-40, 2-30, 2-20, 2-10, 2-5, 2-4, 4-50, 4-40, 4-30, 4-20, 4-10, or 4-5. In some embodiments, the initial ratio of GTP plus GDP to ATP is between about 2× to about 4×. In some embodiments, the initial ratio of GTP plus GDP to CTP is between about 2× to about 4×. In some embodiments, the initial ratio of GTP plus GDP to UTP is between about 2× to about 4×. In some embodiments, the initial ratio of GTP plus GDP to ATP is about 2×. In some embodiments, the initial ratio of GTP plus GDP to CTP is about 2×. In some embodiments, the initial ratio of GTP plus GDP to ATP is about 4×. In some embodiments, the initial ratio of (GTP plus GDP):ATP:CTP:UTP at the start of continuous IVT is 4:2:1:1. In some embodiments, the ratio of (GTP plus GDP):ATP:CTP:UTP at the start of continuous IVT is 4:2:2:1. In some embodiments, the ratio of (GTP plus GDP):ATP:CTP:UTP at the start of continuous IVT is 6:3:3:1.

In some embodiments, a feed solution containing NTPs is added to a continuous IVT reaction mixture to maintain the ratios of NTPs in the IVT reaction vessel at a desired ratio or within a defined range of ratios. In some embodiments, the ratio of NTPs to be maintained in the reaction vessel is the same as the initial ratio of NTPs in the reaction vessel. In some embodiments, feed solution is added to maintain the NTPs at different ratios. The feed solution may comprise NTPs in ratios that are the same or similar to the initial ratios of NTPs in the reaction vessel, or different ratios. In some embodiments, the feed solution comprises NTPs sufficient to maintain the ratio of GTP:ATP:CTP:UTP in the reaction vessel at about 4:2:2:1. In some embodiments, the feed solution comprises NTPs sufficient to maintain the ratio of GTP:ATP:CTP:UTP in the reaction vessel at about 4:2:1:1. In some embodiments, the feed solution comprises NTPs in sufficient concentrations to maintain the total concentration of NTPs in the reaction mixture between 20.0 mM and 100 mM, 25.0 mM and 75.0 mM, 30.0 mM and 50.0 mM, or 35.0 mM and 45.0 mM. The concentration of NTPs suitable for maintaining NTP ratios at a desired ratio or range of concentrations may be determined by multiple methods known in the art. For example, the consumption of each NTP during IVT may be measured, and a feed solution may formulated such that its introduction into the IVT reaction vessel replenishes lost NTPs at a rate similar to their consumption during IVT, thereby maintaining the initial concentrations and ratios of each NTP.

In some embodiments, the feed solution containing NTPs comprises 25-35% GTP, 30-40% CTP, 20-30% ATP, and 10-20% UTP. In some embodiments, the ratio of GTP:ATP in the feed solution is about 0.5-5, 0.5-4, 0.5-3, 0.5-2, 0.5-1, 1-5, 1-4, 1-3, 1-2, 2-5, 2-4, 2-3, 3-5, 3-4, or 4-5. In some embodiments, the ratio of GTP:CTP in the feed solution is about 0.5-5, 0.5-4, 0.5-3, 0.5-2, 0.5-1, 1-5, 1-4, 1-3, 1-2, 2-5, 2-4, 2-3, 3-5, 3-4, or 4-5. In some embodiments, the ratio of GTP:UTP in the feed solution is about 0.5-5, 0.5-4, 0.5-3, 0.5-2, 0.5-1, 1-5, 1-4, 1-3, 1-2, 2-5, 2-4, 2-3, 3-5, 3-4, or 4-5. In some embodiments, the ratio of ATP:CTP in the feed solution is about 0.5-5, 0.5-4, 0.5-3, 0.5-2, 0.5-1, 1-5, 1-4, 1-3, 1-2, 2-5, 2-4, 2-3, 3-5, 3-4, or 4-5. In some embodiments, the ratio of ATP:UTP in the feed solution is about 0.5-5, 0.5-4, 0.5-3, 0.5-2, 0.5-1, 1-5, 1-4, 1-3, 1-2, 2-5, 2-4, 2-3, 3-5, 3-4, or 4-5. In some embodiments, the ratio of CTP:UTP in the feed solution is about 0.5-5, 0.5-4, 0.5-3, 0.5-2, 0.5-1, 1-5, 1-4, 1-3, 1-2, 2-5, 2-4, 2-3, 3-5, 3-4, or 4-5. In some embodiments, the ratio of GTP:ATP in the feed solution is about 1.5-2.5. In some embodiments, the ratio of GTP:ATP in the feed solution is about 1.8. In some embodiments, the ratio of GTP:CTP in the feed solution is about 0.5-2. In some embodiments, the ratio of GTP:CTP in the feed solution is about 0.9. In some embodiments, the ratio of GTP:UTP in the feed solution is about 1.5-2.5. In some embodiments, the ratio of GTP:CTP in the feed solution is about 2.2.

In some embodiments, the feed solution further comprises GDP. In some embodiments, the feed solution containing NTPs comprises 25-35% GTP plus GDP, 30-40% CTP, 20-30% ATP, and 10-20% UTP. In some embodiments, the ratio of GTP plus GDP to ATP in the feed solution is about 0.5-5, 0.5-4, 0.5-3, 0.5-2, 0.5-1, 1-5, 1-4, 1-3, 1-2, 2-5, 2-4, 2-3, 3-5, 3-4, or 4-5. In some embodiments, the ratio of GTP plus GDP to CTP in the feed solution is about 0.5-5, 0.5-4, 0.5-3, 0.5-2, 0.5-1, 1-5, 1-4, 1-3, 1-2, 2-5, 2-4, 2-3, 3-5, 3-4, or 4-5. In some embodiments, the ratio of GTP plus GDP to UTP in the feed solution is about 0.5-5, 0.5-4, 0.5-3, 0.5-2, 0.5-1, 1-5, 1-4, 1-3, 1-2, 2-5, 2-4, 2-3, 3-5, 3-4, or 4-5. In some embodiments, the ratio of ATP plus GDP to CTP in the feed solution is about 0.5-5, 0.5-4, 0.5-3, 0.5-2, 0.5-1, 1-5, 1-4, 1-3, 1-2, 2-5, 2-4, 2-3, 3-5, 3-4, or 4-5. In some embodiments, the ratio of ATP plus GDP to UTP in the feed solution is about 0.5-5, 0.5-4, 0.5-3, 0.5-2, 0.5-1, 1-5, 1-4, 1-3, 1-2, 2-5, 2-4, 2-3, 3-5, 3-4, or 4-5. In some embodiments, the ratio of CTP plus GDP to UTP in the feed solution is about 0.5-5, 0.5-4, 0.5-3, 0.5-2, 0.5-1, 1-5, 1-4, 1-3, 1-2, 2-5, 2-4, 2-3, 3-5, 3-4, or 4-5. In some embodiments, the ratio of GTP plus GDP to ATP in the feed solution is about 1.5-2.5. In some embodiments, the ratio of GTP plus GDP to ATP in the feed solution is about 1.8. In some embodiments, the ratio of GTP plus GDP to CTP in the feed solution is about 0.5-2. In some embodiments, the ratio of GTP plus GDP to CTP in the feed solution is about 0.9. In some embodiments, the ratio of GTP plus GDP to UTP in the feed solution is about 1.5-2.5. In some embodiments, the ratio of GTP plus GDP to CTP in the feed solution is about 2.2.

In some embodiments, the concentration of GTP in the IVT reaction vessel, after addition of the feed solution, is at least 2 times that of any one or more of ATP, CTP, and UTP. In some embodiments, the ratio of GTP:ATP in the IVT reaction vessel, after addition of the feed solution, is 2-50, 2-40, 2-30, 2-20, 2-10, 2-5, 2-4, 4-50, 4-40, 4-30, 4-20, 4-10, or 4-5. In some embodiments, the ratio of GTP:CTP in the IVT reaction vessel, after addition of the feed solution, is 2-50, 2-40, 2-30, 2-20, 2-10, 2-5, 2-4, 4-50, 4-40, 4-30, 4-20, 4-10, or 4-5. In some embodiments, the ratio of GTP:UTP in the IVT reaction vessel, after addition of the feed solution, is 2-50, 2-40, 2-30, 2-20, 2-10, 2-5, 2-4, 4-50, 4-40, 4-30, 4-20, 4-10, or 4-5. In some embodiments, the ratio of GTP:ATP, after addition of the feed solution, is between about 2× to about 4×. In some embodiments, the ratio of GTP:CTP, after addition of the feed solution, is between about 2× to about 4×. In some embodiments, the ratio of GTP:UTP, after addition of the feed solution, is between about 2× to about 4×. In some embodiments, the ratio of GTP:ATP, after addition of the feed solution, is about 2×. In some embodiments, the ratio of GTP:CTP, after addition of the feed solution, is about 2×. In some embodiments, the ratio of GTP:ATP, after addition of the feed solution, is about 4×. In some embodiments, the ratio of GTP:ATP:CTP:UTP, after addition of the feed solution, IVT is 4:2:1:1. In some embodiments, the ratio of GTP:ATP:CTP:UTP, after addition of the feed solution, is 4:2:2:1. In some embodiments, the ratio of GTP:ATP:CTP:UTP, after addition of the feed solution, is 6:3:3:1.

In some embodiments, the concentration of GTP plus GDP in the IVT reaction vessel, after addition of the feed solution, is at least 2 times that of any one or more of ATP, CTP, and UTP. In some embodiments, the ratio of GTP plus GDP to ATP in the IVT reaction vessel, after addition of the feed solution, is 2-50, 2-40, 2-30, 2-20, 2-10, 2-5, 2-4, 4-50, 4-40, 4-30, 4-20, 4-10, or 4-5. In some embodiments, the ratio of GTP plus GDP to CTP in the IVT reaction vessel, after addition of the feed solution, is 2-50, 2-40, 2-30, 2-20, 2-10, 2-5, 2-4, 4-50, 4-40, 4-30, 4-20, 4-10, or 4-5. In some embodiments, the ratio of GTP plus GDP to UTP in the IVT reaction vessel, after addition of the feed solution, is 2-50, 2-40, 2-30, 2-20, 2-10, 2-5, 2-4, 4-50, 4-40, 4-30, 4-20, 4-10, or 4-5. In some embodiments, the ratio of GTP plus GDP to ATP, after addition of the feed solution, is between about 2× to about 4×. In some embodiments, the ratio of GTP plus GDP to CTP, after addition of the feed solution, is between about 2× to about 4×. In some embodiments, the ratio of GTP plus GDP to UTP, after addition of the feed solution, is between about 2× to about 4×. In some embodiments, the ratio of GTP plus GDP to ATP, after addition of the feed solution, is about 2×. In some embodiments, the ratio of GTP plus GDP to CTP, after addition of the feed solution, is about 2×. In some embodiments, the ratio of GTP plus GDP to ATP, after addition of the feed solution, is about 4×. In some embodiments, the ratio of (GTP plus GDP):ATP:CTP:UTP, after addition of the feed solution, IVT is 4:2:1:1. In some embodiments, the ratio of (GTP plus GDP):ATP:CTP:UTP, after addition of the feed solution, is 4:2:2:1. In some embodiments, the ratio of (GTP plus GDP):ATP:CTP:UTP, after addition of the feed solution, is 6:3:3:1.

In some embodiments, the feed solution comprising NTPs further comprises magnesium (Mg2+) ions. Mg2+ ions act as cofactors for RNA polymerase activity, and thus their abundance in an IVT reaction mixture may affect the efficiency of IVT. In some embodiments, the feed solution comprises Mg2+ ions in a concentration sufficient to maintain the Mg2+ ion concentration of the IVT reaction mixture between about 100-800 mM, 150-700 mM, 200-600 mM, 250-500 mM, or 300-450 mM. In some embodiments, the feed solution comprises Mg2+ ions in a concentration sufficient to maintain the Mg2+ ion concentration of the IVT reaction mixture at about 400 mM. In some embodiments, the feed solution comprises Mg2+ ions in a concentration sufficient to maintain the Mg2+ ion concentration of the IVT reaction mixture at about 300 mM. In some embodiments, the feed solution comprises Mg2+ ions in a concentration sufficient to maintain the Mg2+ ion concentration of the IVT reaction mixture at about 200 mM.

In some embodiments, the feed solution comprising NTPs further comprises DNA templates. In some embodiments, the feed solution comprises DNA templates in a concentration sufficient to maintain the DNA template concentration of the IVT reaction mixture between about 0.01-0.1 mg/mL, 0.02-0.08 mg/mL, 0.03-0.07 mg/mL, or 0.04-0.06 mg/mL. In some embodiments, the feed solution comprises DNA templates in a concentration sufficient to maintain the DNA template concentration of the IVT reaction mixture at about 0.05 mg/mL.

In some embodiments, the volume of the continuous IVT reaction mixture is reduced to increase the concentration of DNA templates and RNA polymerases in the IVT reaction vessel. Loss of DNA templates and RNA polymerases during mRNA capture, and addition of feed solution containing NTPs, dilutes the DNA templates and RNA polymerases required for transcription, which may reduce the efficiency of IVT. To counteract dilution of these DNAs and enzymes, the volume of the IVT reaction mixture may be reduced by a method that retains DNA and proteins in the mixture, thereby concentrating the DNA and enzymes. In some embodiments, the IVT reaction mixture is filtered by tangential flow filtration. In some embodiments, the tangential flow filtration comprises using a TFF membrane with a molecular weight cutoff of 500 kDa or less, 200 kDa or less, 150 kDa or less, 100 kDa or less, 50 kDa or less, 40 kDa or less, 30 kDa or less, 20 kDa or less, or lower. In some embodiments, the tangential flow filtration comprises using a TFF membrane with a molecular weight cutoff of 500 kDa or less. In some embodiments, the tangential flow filtration comprises using a TFF membrane with a molecular weight cutoff of 400 kDa or less. In some embodiments, the tangential flow filtration comprises using a TFF membrane with a molecular weight cutoff of 300 kDa or less. In some embodiments, the tangential flow filtration comprises using a TFF membrane with a molecular weight cutoff of 200 kDa or less. In some embodiments, the tangential flow filtration comprises using a TFF membrane with a molecular weight cutoff of 100 kDa or less. In some embodiments, the tangential flow filtration comprises using a TFF membrane with a molecular weight cutoff of 50 kDa or less. In some embodiments, the tangential flow filtration comprises using a TFF membrane with a molecular weight cutoff of 40 kDa or less. In some embodiments, the tangential flow filtration comprises using a TFF membrane with a molecular weight cutoff of 30 kDa or less. In some embodiments, the tangential flow filtration comprises using a TFF membrane with a molecular weight cutoff of 20 kDa or less. In some embodiments, the tangential flow filtration comprises using a TFF membrane with a molecular weight cutoff between 20 kDa and 200 kDa. In some embodiments, the tangential flow filtration comprises using a TFF membrane with a molecular weight cutoff between 20 kDa and 150 kDa.

Removal of liquid from an IVT reaction mixture (e.g., by filtration) reduces the volume of the mixture. Consequently, the concentration of DNA and enzymes is increased after filtration of an IVT reaction mixture. Accordingly, in some embodiments, the concentration of DNA or RNA polymerase in the IVT reaction mixture is increased by filtration. In some embodiments, the concentration of DNA in the IVT reaction mixture is increased by a factor of about 1.1-20, 1.1-15, 1.1-10, 1.1-5, 1.1-4, 1.1-3, 1.1-2, 1.1-1.5, 1.2-20, 1.2-15, 1.2-10, 1.2-5, 1.2-4, 1.2-3, 1.2-2, 1.2-1.5, 1.5-20, 1.5-15, 1.5-10, 1.5-5, 1.5-4, 1.5-3, 1.5-2, 2-20, 2-15, 2-10, 2-5, 2-4, or 2-3, relative to the concentration before filtration of the IVT reaction mixture. In some embodiments, the concentration of RNA polymerase in the IVT reaction mixture is increased by a factor of about 1.1-20, 1.1-15, 1.1-10, 1.1-5, 1.1-4, 1.1-3, 1.1-2, 1.1-1.5, 1.2-20, 1.2-15, 1.2-10, 1.2-5, 1.2-4, 1.2-3, 1.2-2, 1.2-1.5, 1.5-20, 1.5-15, 1.5-10, 1.5-5, 1.5-4, 1.5-3, 1.5-2, 2-20, 2-15, 2-10, 2-5, 2-4, or 2-3, relative to the concentration before filtration of the IVT reaction mixture.

In some embodiments, one or more eluates comprising RNA are added to the IVT reaction vessel before a DNase or protease is added to the IVT reaction vessel or mixture. In other embodiments, one or more eluates comprising RNA are combined separately from the IVT reaction vessel or mixture.

In some embodiments, the in vitro transcribed mRNA is produced by a method comprising the steps of:
(i) in a reaction vessel comprising a mixture comprising a DNA molecule and an RNA polymerase, in vitro transcribing a DNA molecule, whereby the RNA polymerase transcribes the DNA molecule to produce an mRNA, wherein mRNA is removed from the reaction vessel by the steps of:
   (1) transferring a portion of the mixture from the reaction vessel to a column comprising a stationary phase;
   (2) passing the portion of the mixture through the column, whereby the stationary phase retains mRNA from the mixture; and
   (3) re-introducing a flowthrough from the column into the reaction vessel, wherein the concentration of mRNA in the flowthrough of step (3) is lower than the concentration of mRNA in the portion of the mixture of step (1).

In some aspects, the disclosure relates to a method of removing in vitro transcribed mRNA from an in vitro transcription reaction, the method comprising:
(i) in a reaction vessel comprising a mixture comprising a DNA molecule and an RNA polymerase, in vitro transcribing a DNA molecule, whereby the RNA polymerase transcribes the DNA molecule to produce an RNA, wherein RNA is removed from the reaction vessel by the steps of:
   (1) transferring a portion of the mixture from the reaction vessel to a column comprising a stationary phase;
   (2) passing the portion of the mixture through the column, whereby the stationary phase retains RNA from the mixture; and
   (3) re-introducing a flowthrough from the column into the reaction vessel, wherein the concentration of RNA in the flowthrough of step (3) is lower than the concentration of RNA in the portion of the mixture of step (1); and
(ii) isolating the mRNA from the mixture.

In some embodiments, the method further comprises, prior to the isolation of step (ii), contacting the mixture with a DNase, and incubating the mixture for a period of time sufficient for the DNase to cleave one or more DNAs in the mixture to produce DNA fragments.

### Salt Intensification of Column Chromatography

Some aspects of the disclosure relate to methods of purifying mRNA compositions by adding a high-salt buffer to the mRNA composition to increase the salt concentration, adding the composition to a stationary phase to bind mRNA to the stationary phase, and eluting bound mRNA from the stationary phase to obtain eluted mRNA. Addition of a high-salt buffer promotes formation of compact secondary structures by mRNAs, but leaving the poly(A) tail exposed. Exposure of the poly(A) tail allows mRNAs to bind to stationary phases such as oligo-dT stationary phase, but the compact structure induced by high salt concentrations reduces steric interactions in which a portion of a stationary phase-bound first mRNA prevents a second mRNA from binding to the stationary phase. Thus, adding a high-salt mRNA composition to a stationary phase causes more mRNA molecules to bind to the stationary phase than addition of an equivalent mRNA composition with a lower salt concentration. However, mRNA must be dissolved in a solution in order to pass through a stationary phase. The benefits of high salt concentrations for increasing binding of mRNA to stationary phase must therefore be balanced with the risk of mRNA precipitation. Surprisingly, precipitation requires an extended amount of time even at high salt concentrations. Accordingly, addition of a high-salt buffer to an mRNA composition to produce a high-salt mRNA composition followed by adding the high-salt mRNA composition to a stationary phase shortly thereafter, allows the benefits of high salt concentrations for stationary phase binding to be realized without a consequent reduction in yield due to mRNA precipitation.

In some embodiments, the method comprises heating the RNA composition to denature RNA before the high-salt buffer is added (i.e., the high-salt buffer is added to a composition comprising denatured RNA). Denaturation disrupts the secondary structure of nucleic acids (e.g., mRNAs), promoting release of bound impurities and the formation of linear nucleic acid structures. RNA may be denatured using any method. In some embodiments, RNA is denatured by heating an mRNA composition before the high-salt buffer is added. In some embodiments, RNA is denatured by heating the mRNA composition after the high-salt buffer is added. In some embodiments, RNA is denatured by heating the RNA composition to 60° C. to 90° C., 60° C. to 80° C., 60° C. to 70° C., 65° C. to 85° C., 65° C. to 75° C., 70° C. to 90° C., 70° C. to 80° C., 65° C. to 70° C., 70° C. to 75° C., or 75° C. to 95° C. In some embodiments, the RNA composition is heated for less than 2 minutes, less than 90 seconds, less than 1 minute, less than 50 seconds, less than 40 seconds, less than 30 seconds, less than 20 seconds, or less than 10 seconds. In some embodiments, the RNA composition is heated for 5-90 seconds, 5-60 seconds, 5-30 seconds, 5-seconds, 10-60 seconds, 10-30 seconds, 20-60 seconds, 20-40 seconds, 30-90 seconds, 30-60 seconds, 40-90 seconds, 40-60 seconds, 60-120 seconds, 60-90 seconds, or 90-120 seconds. In some embodiments, the high-salt RNA composition is heated in the presence of a denaturant molecule (e.g., a chemical small molecule that destabilizes or denatures RNA). A denaturant molecule may include dimethyl sulfoxide (e.g., at a concentration of 0.05-1% v/v, 0.1-0.5% v/v, 0.05-0.5% v/v, or 0.25-0.75% v/v), guanidine (e.g., at a concentration of 50-250 mM, 100-500 mM, 250-1000 mM, 1-8 M, 2-6 M, 3-5 M, or 5-8 M), or urea (e.g., at a concentration of 50-250 mM, 100-500 mM, 250-1000 mM, 1-8 M, 2-6 M, 3-5 M, or 5-8 M).

In some embodiments, a change in the relative amount of denatured RNA in an RNA composition during a denaturation process (e.g., heating the RNA composition to 60° C. to 90° C., 60° C. to 80° C., 60° C. to 70° C., 65° C. to 85° C., 65° C. to 75° C., 70° C. to 90° C., 70° C. to 80° C., 65° C. to 70° C., 70° C. to 75° C., or 75° C. to 95° C.) is determined by hyperchromicity curves (e.g., spectroscopic melting curves). In some embodiments, a change in the relative amount of denatured RNA is determined by measuring the change in secondary structure of the total RNA in a composition (e.g., by determining a change in ultraviolet absorption). In some embodiments, a change in the relative amount of denatured RNA is determined by monitoring the change in secondary structure of the total RNA in a composition (e.g., by determining a change in ultraviolet absorption) before and after the denaturation process (e.g., heating the RNA composition to 60° C. to 90° C., 60° C. to 80° C., 60° C. to 70° C., 65° C. to 85° C., 65° C. to 75° C., 70° C. to 90° C., 70° C. to 80° C., 65° C. to 70° C., 70° C. to 75° C., or 75° C. to 95° C.). In some embodiments, at least 50%, 55%, 60%, 65%, 70%, 75%, 80%, or 85% of the total RNA in a denatured RNA composition comprises denatured RNA. In some embodiments, at least 90% (e.g., at least 91%, 92%, 93%, 94%, 95%, 96%, 97%, 98%, 99% or 100%) of the total RNA in a denatured RNA composition comprises denatured RNA. In some embodiments, 50-70%, 45-60%, 55-70%, 60-80%, 60-100%, 75-100%, 50-95%, 75-95%, 80-100%, 80-90%, 90-95%, 95-100%, 90-99%, or 95-99% of the total RNA in a denatured RNA composition comprises denatured RNA. In some embodiments, the relative amount of denatured RNA in a denatured RNA composition is determined by hyperchromicity curves (e.g., spectroscopic melting curves). Hyperchromicity, the property of nucleic acids such as RNA to exhibit an increase in extinction coefficient upon the loss of structure during heating, may be measured (e.g., during denaturation of RNA, e.g., by heating) using a spectrophotometer. In some embodiments, the extinction coefficient of RNA is measured at 205 nm, 220 nm, 260 nm, or 200-300 nm. In some embodiments, the relative amount of denatured RNA in a denatured RNA composition is determined using a method as described in S. J. Schroeder and D. H. Turner, “Optical melting measurements of nucleic acid thermodynamics”, Methods Enzymol. 468 (2009) 371-387; or Gruenwedel, D. W., “Nucleic Acids: Properties and Determination”, Encyclopedia of Food Sciences and Nutrition, 2003, Pages 4147-4152.

In some embodiments, the high-salt buffer is added to the RNA composition by in-line mixing. In some embodiments, in-line mixing refers to mixing of a first continuous stream of a solution with a second continuous stream of a solution. In some embodiments, the first and second continuous streams are controlled by independent pumps (e.g., independent peristaltic pumps). In some embodiments, in-line mixing relies on flow control conditions, for example, process flow conditions wherein flow parameters (e.g., flow rate, temperature) are controlled by a flow regulating device comprising at least one pump system. In some embodiments, the first continuous stream is a high-salt buffer (e.g., comprising at least 100 mM salt), and the second continuous stream is a composition comprising RNA. In some embodiments, the RNA composition has been desalted (e.g., is a low-salt RNA composition) and/or comprises denatured RNA. In-line mixing typically occurs shortly prior to binding a composition comprising RNA to a stationary phase. In some embodiments, in-line mixing occurs for less than 2 minutes, less than 90 seconds, less than 1 minute, less than 50 seconds, less than 40 seconds, less than 30 seconds, less than 20 seconds, or less than 10 seconds. In some embodiments, in-line mixing occurs for 5-90 seconds, 5-60 seconds, 5-30 seconds, 5-15 seconds, 10-60 seconds, 10-30 seconds, 20-60 seconds, 20-40 seconds, 30-90 seconds, 30-60 seconds, 40-90 seconds, 40-60 seconds, 60-120 seconds, 60-90 seconds, or 90-120 seconds. In some embodiments, in-line mixing occurs at a temperature of 4° C. to 30° C., 4° C. to 25° C., 4° C. to 20° C., 4° C. to 15° C., 4° C. to 10° C., 4° C. to 8° C., 10° C. to 30° C., 10° C. to 25° C., 10° C. to 20° C., 10° C. to 15° C., or 15° C. to 25° C.

In some embodiments, the high-salt buffer is added to the RNA composition by bolus addition. In contrast to in-line mixing in which two liquid streams are combined continuously, bolus addition involves the discrete addition of one composition to another. Bolus addition may occur over several seconds or minutes, and be followed by mixing to incorporate the high-salt buffer throughout the RNA composition, thereby distributing salts of the high-salt buffer throughout the RNA composition.

In some embodiments, the high-salt buffer is added to the RNA composition 5-90 seconds, 5-60 seconds, 5-30 seconds, 5-15 seconds, 10-60 seconds, 10-30 seconds, 20-60 seconds, 20-40 seconds, 30-90 seconds, 30-60 seconds, 40-90 seconds, 40-60 seconds, 60-120 seconds, 60-90 seconds, or 90-120 seconds prior to binding a composition comprising RNA to a stationary phase. In some embodiments, adding the high-salt buffer occurs 1-60 minutes, 1-45 minutes, 1-30 minutes, 1-25 minutes, 1-20 minutes, 1-15 minutes, 1-10 minutes, 1-5 minutes, or 1-2 minutes prior to adding the high-salt RNA composition to the stationary phase. In some embodiments, adding the high-salt RNA composition to the stationary phase occurs within 1 hour or less, 45 minutes or less, 30 minutes or less, 25 minutes or less, 20 minutes or less, 15 minutes or less, 10 minutes or less, 9 minutes or less, 8 minutes or less, 7 minutes or less, 6 minutes or less, 5 minutes or less, 4 minutes or less, 3 minutes or less, 2 minutes or less, or 1 minute or less of adding the high-salt buffer to the RNA composition. In some embodiments, the high-salt buffer, the RNA composition, and/or the high-salt RNA composition produced by adding the high-salt buffer has a temperature of 4° C. to 30° C., 4° C. to 25° C., 4° C. to 20° C., 4° C. to 15° C., 4° C. to 10° C., 4° C. to 8° C., 10° C. to 30° C., 10° C. to 25° C., 10° C. to 20° C., 10° C. to 15° C., or 15° C. to 25° C.

In some embodiments, a high-salt buffer (e.g., that may be mixed with an RNA composition) comprises a salt concentration of at least 50 mM, at least 60 mM, at least 70 mM, at least 80 mM, at least 90 mM, at least 100 mM, at least 125 mM, at least 150 mM, at least 200 mM, at least 250 mM, at least 300 mM, at least 350 mM, at least 400 mM, at least 500 mM, at least 600 mM, at least 700 mM, at least 800 mM, at least 900 mM, or at least 1000 mM. In some embodiments, a high-salt buffer comprises a salt concentration of 50-500 mM, 50-250 mM, 50-100 mM, 50-75 mM, 60-150 mM, 75-500 mM, 75-200 mM, 100-500 mM, 100-250 mM, 150-350 mM, 200-400 mM, 250-500 mM, 300-400 mM, 350-450 mM, 400-500 mM, 400-600 mM, 500-700 mM, 500-750 mM, 700-1000 mM, 750-900 mM, or 850-1000 mM. In some embodiments, a high-salt buffer comprises a salt concentration of 1-2 M, 2-3 M, 3-4 M, or 4-5 M. In some embodiments, a high-salt buffer comprises a conductivity of at least 5 mS/cm, at least 6 mS/cm, at least 7 mS/cm, at least 8 mS/cm, at least 9 mS/cm, at least 10 mS/cm, at least 12 mS/cm, or at least 15 mS/cm. In some embodiments, a high-salt buffer comprises a conductivity of 5-10 mS/cm, 5-15 mS/cm, 5-7 mS/cm, 6-9 mS/cm, 8-10 mS/cm, 9-12 mS/cm, or 10-15 mS/cm.

In some embodiments, mixing a composition comprising RNA with a high-salt buffer produces a high-salt RNA composition comprising RNA and salt at a concentration of at least 100 mM. In some embodiments, mixing a composition comprising RNA with a high-salt buffer produces a composition comprising RNA and salt at a concentration of 100-500 mM, 100-250 mM, 150-350 mM, 200-400 mM, 250-500 mM, 300-400 mM, 350-450 mM, 400-500 mM, 400-600 mM, 500-700 mM, 500-750 mM, 700-1000 mM, 750-900 mM, or 850-1000 mM. In some embodiments, the salt concentration of the high-salt RNA composition is at least 100 mM, at least 200 mM, at least 300 mM, at least 400 mM, at least 500 mM, at least 600 mM, at least 700 mM, at least 800 mM, at least 900 mM, at least 1 M, or more. In some embodiments, the high-salt RNA composition has a salt concentration of about 100 mM to about 200 mM, about 200 mM to about 400 mM, about 400 mM to about 600 mM, about 600 mM to about 800 mM, about 800 mM to about 1 M, about 1 M to about 1.5 M, about 1.5 M to about 2 M, about 2 M to about 2.5 M, about 2.5 M to about 3 M, about 3 M to about 4 M, or about 4 M to about 5 M. In some embodiments, the high-salt mRNA composition has a salt concentration of about 400 mM to about 600 mM. In some embodiments, the high-salt RNA composition comprises a salt concentration of about 500 mM. In some embodiments, the salt concentration of the high-salt RNA composition refers to the concentration of sodium chloride, potassium chloride, ammonium chloride, ammonium sulfate, monosodium phosphate, disodium phosphate, or trisodium phosphate in the composition. In some embodiments, mixing a composition comprising RNA with a high-salt buffer produces a composition comprising RNA and salt having a conductivity of less than 2 mS/cm. In some embodiments, mixing a composition comprising RNA with a high-salt buffer produces a composition comprising RNA and salt having a conductivity of 2-5 mS/cm, 2-7 mS/cm, 5-10 mS/cm, 5-15 mS/cm, 5-7 mS/cm, 6-9 mS/cm, 8-10 mS/cm, 9-12 mS/cm, or 10-15 mS/cm.

In some embodiments, a buffer (e.g., a low-salt or high-salt buffer) comprises NaCl, KCl, LiCl, NaH₂PO₄, Na₂HPO₄, or Na₃PO₄. In some embodiments, a buffer (e.g., a low-salt or high-salt buffer) comprises any source of sodium, potassium, magnesium, phosphate, chloride, or any other source of salt ions. In some embodiments, a buffer (e.g., a low-salt or high-salt buffer) may further comprise a buffering agent in order to maintain a consistent pH. In some embodiments, a buffer (e.g., a low-salt or high-salt buffer) comprises a neutral pH. In some embodiments, a buffer (e.g., a low-salt or high-salt buffer) comprises a pH of about 6, about 6.5, about 7, about 7.4, about 8, or about 6-8. Examples of buffering agents for use herein include ethylenediamine tetraacetic acid (EDTA), succinate, citrate, aspartic acid, glutamic acid, maleate, cacodylate, 2-(N-morpholino)-ethanesulfonic acid (MES), N-(2-acetamido)-2-aminoethanesulfonic acid (ACES), piperazine-N,N′-2-ethanesulfonic acid (PIPES), 2-(N-morpholino)-2-hydroxy-propanesulfonic acid (MOPSO), N,N-bis-(hydroxyethyl)-2-aminoethanesulfonic acid (BES), 3-(N-morpholino)-propanesulfonic acid (MOPS), N-2-hydroxyethyl-piperazine-N-2-ethanesulfonic acid (HEPES), 3-(N-tris-(hydroxymethyl)methylamino)-2-hydroxypropanesulfonic acid (TAPSO), 3-(N,N-bis[2-hydroxyethyl]amino)-2-hydroxypropanesulfonic acid (DIPSO), N-(2-hydroxyethyl)piperazine-N′-(2-hydroxypropanesulfonic acid) (HEPPSO), 4-(2-hydroxyethyl)-1-piperazine propanesulfonic acid (EPPS), N-[tris(hydroxymethyl)-methyl]glycine (Tricine), N,N-bis(2-hydroxyethyl)glycine (Bicine), [(2-hydroxy-1,1-bis(hydroxymethyl)ethyl)amino]-1-propanesulfonic acid (TAPS), N-(1,1-dimethyl-2-hydroxyethyl)-3-amino-2-hydroxypropanesulfonic acid (AMPSO), tris(hydroxymethyl)aminomethane (Tris), and bis[2-hydroxyethyl]iminotris-[hydroxymethyl]methane (Bis-Tris). Other buffers compositions, buffer concentrations, and additional components of a solution for use herein will be apparent to those skilled in the art.

In some embodiments, mixing comprises cooling of a composition comprising RNA to a temperature of 4° C. to 30° C., 4° C. to 25° C., 4° C. to 20° C., 4° C. to 15° C., 4° C. to 10° C., 4° C. to 8° C., 10° C. to 30° C., 10° C. to 25° C., 10° C. to 20° C., 10° C. to 15° C., or 15° C. to 25° C. In some embodiments, mixing comprises cooling of a composition comprising RNA to a temperature below 60° C., 55° C., 50° C., 45° C., 40° C., 35° C., 30° C., 25° C., 20° C., 15° C., 10° C., or 5° C. In some embodiments, cooling occurs simultaneously with mixing of a composition comprising RNA and low salt buffer with a high salt buffer. In some embodiments, during cooling, a composition comprising RNA is maintained at a total salt concentration of less than 20 mM, less than 15 mM, less than 10 mM, less than 5 mM, or less than 1 mM. In some embodiments, during cooling, a composition comprising RNA is maintained at a total salt concentration of 1-20 mM, 1-15 mM, 1-10 mM, 1-5 mM, 5-20 mM, 5-10 mM, 10-20 mM, 10-15 mM or 15-20 mM. In some embodiments, cooling occurs simultaneously with mixing of a composition comprising RNA and low salt buffer with a high salt buffer. In some embodiments, during cooling, a composition comprising RNA is maintained at less than 2.5 mS/cm, less than 2 mS/cm, less than 1.5 mS/cm, less than 1 mS/cm, or less than 0.5 mS/cm. In some embodiments, cooling occurs simultaneously with mixing of a composition comprising RNA and low salt buffer with a high salt buffer. In some embodiments, during cooling, a composition comprising RNA is maintained at 0.1-2.5 mS/cm, 0.1-2 mS/cm, 0.5-2 mS/cm, 0.5-1 mS/cm, 1-2 mS/cm, 1-1.5 mS/cm, or 1-1.25 mS/cm.

Some embodiments of the methods provided herein involve the production of high-salt RNA compositions in which most or all RNA molecules are dissolved in solution. In some embodiments, the high-salt RNA composition comprises at least 2.0 g/L, 2.5 g/L, 3.0 g/L, 3.5 g/L, 4.0 g/L, 4.5 g/L, 5.0 g/L, 6.0 g/L, 7.0 g/L, 8.0 g/L, 9.0 g/L, 10.0 g/L, or more dissolved RNA. In some embodiments, the high-salt RNA composition comprises about 2.0 g/L to about 4.0 g/L, about 4.0 g/L to about 6.0 g/L, about 6.0 g/L to about 8.0 g/L, or about 8.0 g/L to about 10.0 g/L dissolved RNA. In some embodiments, the high-salt RNA composition comprises about 4.0 g/L to about 6.0 g/L dissolved RNA. In some embodiments, at least at least 80%, at least 85%, at least 90%, at least 95%, at least 97%, at least 98%, at least 99%, or up to 100% of RNAs in the high-salt mRNA composition are dissolved mRNAs. In some embodiments, the concentration of precipitated (i.e., solid) RNA in the composition is 0.5 g/L or less, 0.4 g/L or less, 0.3 g/L or less, 0.2 g/L or less, 0.1 g/L or less, or 0.05 g/L or less. In some embodiments, the high-salt RNA composition does not comprise precipitated RNA. Methods of measuring the mass of precipitated RNA in a composition are generally known in the art, and include separation and weighing of the precipitate, or desalting and resolubilizing the precipitate to quantify the amount of RNA by spectroscopy. In some embodiments, the percentage of RNAs in a composition that are dissolved RNAs is calculated by determining the mass of precipitated RNA in the composition, determining the mass of dissolved RNA in the composition, and calculating the percentage of all RNA in the composition that is precipitated RNA. In some embodiments, absence of a precipitate, or absence of RNA in the precipitate, indicates that all RNAs in the composition are dissolved RNAs.

Some embodiments of the methods herein involve binding (i.e., contacting) compositions comprising RNA to a stationary phase. In some embodiments, methods herein comprise binding compositions comprising RNA to the stationary phase following mixing of RNA compositions with high-salt buffers.

In some embodiments, the stationary phase comprises fiber, particles, resin, and/or beads, Examples of stationary phases include but are not limited to resin, silica (e.g., alkylated and non-alkylated silica), polystyrenes (e.g., alkylated and non-alkylated polystyrenes), polystyrene divinylbenzenes, etc. In some embodiments, a stationary phase comprises particles, for example porous particles. In some embodiments, a stationary phase (e.g., particles of a stationary phase) is hydrophobic (e.g., made of an intrinsically hydrophobic material, such as polystyrene divinylbenzene), or comprise hydrophobic functional groups. In some embodiments, a stationary phase is a membrane or monolithic stationary phase. A monolithic stationary phase is a continuous, unitary, porous structure prepared by in situ polymerization or consolidation inside the column tubing. In some embodiments, the surface is functionalized to convert it into a sorbent with the desired chromatographic binding properties.

The particle size (e.g., as measured by the diameter of the particle) of an HPLC stationary phase can vary. In some embodiments, the particle size of a HPLC stationary phase ranges from about 1 μm to about 100 μm (e.g., any value between 1 and 100, inclusive) in diameter. In some embodiments, the particle size of a HPLC stationary phase ranges from about 2 μm to about 10 μm, about 2 μm to about 6 μm, or about 4 μm in diameter. The pore size of particles (e.g., as measured by the diameter of the pore) can also vary. In some embodiments, the particles comprise pores having a diameter of about 100A to about 10,000A. In some embodiments, the particles comprise pores having a diameter of about 100A to about 5000A, about 100A to about 1000A, or about 1000A to about 2000A. In some embodiments, the stationary phase comprises polystyrene divinylbenzene, for example as used in PLRP-S 4000 columns or DNAPac-RP columns.

In some embodiments, the stationary phase comprises a hydrophobic interaction chromatography (HIC) stationary phase, such as an HIC resin. Some embodiments of the methods described here may comprise any HIC resin. In some embodiments, the HIC resin comprises one or more butyl, phenyl, octyl, t-butyl, methyl, and/or ethyl functional groups. In some embodiments, the HIC resin is a HiTrap Butyl HP resin, CaptoPhenyl resin, Phenyl Sepharose 6 resin, Phenyl Sepharose™ High Performance resin, Octyl Sepharose™ High Performance resin, Fractogel™ EMD Propyl resin, Fractogel™ EMD Phenyl resin, Macro-Prep™ Methyl resin, HiScreen Butyl FF, HiScreen Octyl FF, or Tosoh Hexyl. In some embodiments, the HIC resin is a (poly)styrene-divinylbenzene (PS-DVB) R150 bead resin with 2000 Angstrom pores.

In some embodiments, the stationary phase comprises a capture nucleic acid (e.g. oligonucleotide or polynucleotide) that comprises a nucleotide sequence that is complementary to a nucleotide sequence of an mRNA of the high-salt RNA composition. Watson-Crick base pairing between the capture nucleic acid and the complementary sequence on the mRNA in the high-salt RNA composition promote retention of the mRNA by the stationary phase, while nucleic acids that lack the complementary sequence pass over the stationary phase due to lack of hybridization to the capture nucleic acid. In some embodiments, the capture nucleic acid is a DNA. In other embodiments, the capture nucleic acid is an RNA. In some embodiments, the capture nucleic acid comprises a nucleotide sequence that is 5-200, 10-50, 10-100, 50-200, 100-150, or 125-200 nucleotides in length that is complementary to a nucleotide sequence of an mRNA in the high-salt composition. In some embodiments, the capture nucleic acid is 5-200, 10-50, 10-100, 50-200, 100-150, or 125-200 nucleotides in length. In some embodiments, the capture nucleic acid is linked to a bead and/or resin of the stationary phase by a linker.

In some embodiments, the stationary phase comprises oligo-dT resin. In some embodiments, the oligo-dT resin is a (poly)styrene-divinylbenzene (PS-DVB) bead resin with 2000 Angstrom pores derivatized with poly dT. In some embodiments, poly dT comprises 5-200, 10-50, 10-100, 50-200, 100-150, or 125-200 thymidines and/or uracils. In some embodiments, poly dT comprises 20 thymidines in length. In some embodiments, poly dT is linked directly to the bead resin. In some embodiments, poly dT is linked to the bead resin via a linker.

In some embodiments, the stationary phase is equilibrated with a buffer prior to binding the RNA to the stationary phase. In some embodiments, the stationary phase is equilibrated with a buffer comprising 100 mM NaCl, 10 mM Tris, and 1 mM EDTA at pH 7.4. In some embodiments, the stationary phase is washed with a buffer after the RNA is bound to the stationary phase. In some embodiments, the washing step comprises a buffer comprising 100 mM NaCl, 10 mM Tris, and 1 mM EDTA, at pH 7.4.

In some embodiments, the binding of the RNA to the stationary phase occurs at a temperature of lower than 40° C. In some embodiments, the binding of the RNA to the stationary phase occurs at a temperature of 4° C. to 30° C., 4° C. to 25° C., 4° C. to 20° C., 4° C. to 15° C., 4° C. to 10° C., 4° C. to 8° C., 10° C. to 30° C., 10° C. to 25° C., 10° C. to 20° C., 10° C. to 15° C., or 15° C. to 25° C.

In some embodiments, the high-salt RNA composition comprising RNA is bound to or in contact with the stationary phase for a total residence time of less than 20 minutes, less than 18 minutes, less than 15 minutes, less than 12 minutes, less than 10 minutes, less than 9 minutes, less than 8 minutes, less than 7 minutes, less than 6 minutes, less than 5 minutes, less than 4 minutes, less than 3 minutes, less than 2 minutes, or less than 1 minute. In some embodiments, the composition comprising RNA is bound to or in contact with the stationary phase for a total residence time of 1-2, 1-5, 2-5, 2-10, 5-20, 5-10, 5-15, 8-15, 10-15, 12-20, or 15-20 minutes.

In some embodiments, the stationary phase is comprised in a column. In some embodiments, the concentration of RNA in the high-salt RNA composition is less than 100%, less than 90%, less than 80%, less than 70%, less than 60%, or less than 50% of the dynamic binding capacity of the column. Dynamic binding capacity refers to the amount or concentration of an analyte (e.g., RNA) that can be passed through a column without significant breakthrough of the analyte. Contacting a column or stationary phase with a composition comprising an analyte concentration that exceeds the dynamic binding capacity causes analyte to pass through the stationary phase without being bound, reducing analyte yield. In some embodiments, at least 70%, at least 75%, at least 80%, at least 85%, at least 90%, at least 91%, at least 92%, at least 93%, at least 94%, at least 95%, at least 96%, at least 97%, at least 98%, at least 99%, or up to 100% of mRNAs in the high-salt RNA composition bind to the stationary phase.

In some embodiments, addition of a high-salt buffer prior to binding the RNA to the stationary phase increases the productivity of the method. “Productivity,” as used herein, refers to a quantitative value of output product (i.e., nucleic acid) obtained from the process. More specifically, productivity is the quantitative measure of nucleic acid purified using a given volume of stationary phase in a given length of time, expressed in units of (mass)−(volume of stationary phase)⁻¹·(time)⁻¹ (e.g., g·L⁻¹·hr⁻¹). In some embodiments, the productivity of the method is a least about 0.25 g/L·hr, optionally wherein the productivity of the method is about 0.5 g/L·hr, about 0.75 g/L·hr, about 3 g/L·hr, about 4 g/L·hr, about 5 g/L·hr, about 6 g/L·hr, about 7 g/L·hr, about 8 g/L·hr, about 9 g/L·hr, about 10 g/L·hr, or more. In some embodiments, the total volume of stationary phase comprised within the column is between about 0.1 L to about 100 L. In some embodiments, the total volume of stationary phase is about 0.1 L to about 0.5 L, 0.5 L to about 2 L, about 2 L to about 4 L, about 4 L to about 6 L, about 6 L to about 8 L, about 8 L to about 10 L, about 10 L to about 15 L, about 15 L to about 20 L, about 20 L to about 25 L, about 25 L to about 30 L, about 30 L to about 40 L, about 40 L to about 50 L, about 50 L to about 75 L, or about 75 L to about 100 L.

In some embodiments, the methods comprise eluting RNA from the stationary phase. In some embodiments, the RNA is eluted from the stationary phase using water or a buffer (e.g., a buffer comprising 10 mM Tris, 1 mM EDTA, at pH 8.0).

In some embodiments, the RNA eluted from the stationary phase comprises at least 50%, 55%, 60%, 65%, 70%, 75%, 80%, or 85% poly-A tailed RNA. In some embodiments, the RNA eluted from the stationary phase comprises about 20-100%, 20-90%, 20-80%, 20-70%, 20-60%, 20-50%, 20-40%, 20-30%, 25-75%, 30-50%, 40-60%, 50-70%, 45-60%, 55-70%, 60-80%, 60-100%, 75-100%, 50-95%, 75-95%, 80-100%, 80-90%, 90-95%, 95-100%, 90-99%, or 95-99% poly-A tailed mRNA. In some embodiments, the RNA eluted from the stationary phase comprises at least 91%, 92%, 93%, 94%, 95%, 96%, 97%, 98%, 99% or 100% poly-A tailed mRNA.

In some embodiments, mixtures comprising RNA are desalted to produce low-salt RNA compositions (e.g., having less than 50 mM total salt concentration). In some embodiments, a mixture comprising RNA (e.g., a mixture produced by an IVT reaction) is desalted prior to denaturation of the RNA and/or mixing with a high-salt buffer. In some embodiments, a desalting occurs after denaturation (e.g., by heating), but before addition of the high-salt buffer. In some embodiments, desalting occurs before denaturation, and the low-salt mRNA composition is heated to denature the mRNA. In some embodiments, the RNA composition is desalted, but not denatured, before addition of the high-salt buffer.

In some embodiments, a low-salt RNA composition comprises sodium, potassium, magnesium, manganese, calcium, sulfate, phosphate, and/or chloride salts. In some embodiments, a low-salt RNA composition comprises sodium chloride, sodium phosphate, sodium sulfate, potassium chloride, potassium phosphate, potassium sulfate, magnesium chloride, magnesium phosphate, magnesium sulfate, calcium chloride, calcium phosphate, and/or calcium sulfate. In some embodiments, a low-salt RNA composition comprises a total salt concentration of less than 20 mM, less than 15 mM, less than 10 mM, less than 5 mM, or less than 1 mM. In some embodiments, a low-salt RNA composition comprises a salt concentration of 1-20 mM, 1-15 mM, 1-10 mM, 1-5 mM, 5-20 mM, 5-10 mM, 10-20 mM, 10-15 mM or 15-20 mM. In some embodiments, a low-salt RNA composition results in a conductivity of less than 2.5 mS/cm, less than 2 mS/cm, less than 1.5 mS/cm, less than 1 mS/cm, or less than 0.5 mS/cm. In some embodiments, a low-salt RNA composition comprises a conductivity of 0.1-2.5 mS/cm, 0.1-2 mS/cm, 0.5-2 mS/cm, 0.5-1 mS/cm, 1-2 mS/cm, 1-1.5 mS/cm, or 1-1.25 mS/cm.

In some embodiments, desalting a mixture comprising RNA is accomplished by binding the RNA to a hydrophobic interaction chromatography (HIC) resin and eluting the RNA from the HIC resin to produce the low-salt RNA composition. In some embodiments, the HIC resin is equilibrated with a buffer prior to binding the RNA to the resin. In some embodiments, the HIC resin is equilibrated with a buffer comprising 100 mM NaCl, 10 mM Tris, 1 mM EDTA pH 7.4. In some embodiments, the RNA is eluted from the HIC resin using water or a buffer. Some embodiments of the methods described here may comprise any HIC resin. In some embodiments, the HIC resin comprises butyl, phenyl, octyl, t-butyl, methyl, and/or ethyl functional groups. In some embodiments, the HIC resin is a HiTrap Butyl HP resin, CaptoPhenyl resin, Phenyl Sepharose 6 resin, Phenyl Sepharose™ High Performance resin, Octyl Sepharose™ High Performance resin, Fractogel™ EMD Propyl resin, Fractogel™ EMD Phenyl resin, Macro-Prep™ Methyl resin, HiScreen Butyl FF, HiScreen Octyl FF, or Tosoh Hexyl. In some embodiments, the HIC resin is a (poly)styrene-divinylbenzene (PS-DVB) R150 bead resin with 2000 Angstrom pores.

In some embodiments, desalting a mixture comprising RNA is accomplished by dilution of the mixture with water (e.g., a 10× water dilution), tangential flow filtration (TFF) of the mixture into water, or ambient oligo-dT (i.e., under native, non-denaturing RNA conditions). In some embodiments, desalting a mixture comprising RNA is accomplished by tangential flow filtration.

In some embodiments, an RNA composition (e.g., a low-salt RNA composition) is denatured. In some embodiments, a low-salt RNA composition is denatured prior to (e.g., immediately prior to) mixing with a high-salt buffer and subsequent binding of the denatured RNA to a stationary phase.

### Purification Combinations

Also provided are combinations of various purification methods. Exemplary purification methods that can be combined (e.g., sequentially) include protease digestion, salt precipitation, removal of mRNA during IVT, desalting with low-salt solutions, heat denaturation, chemical denaturation, adding a high-salt buffer, in-line mixing with one or more solutions, tangential flow filtration, oligo dT affinity chromatography, denatured oligo dT affinity chromatography, hydrophobic interaction chromatography (HIC), and any combination thereof. Some embodiments comprise using a combination of the above-mentioned purification techniques, wherein each technique is independently used 0, 1, 2, 3, 4, 5, or more times and the techniques are used in any possible order.

mRNA Compositions

Some aspects of the disclosure relate to compositions comprising RNA produced by any of the methods described herein. In some embodiments, the concentration of proteins in the RNA composition produced by the method is 0.8% (% w/w) or less, 0.6% or less, 0.4% or less, or 0.2% or less. Methods of measuring the amount of protein in a sample include colorimetric assays (e.g., Bradford assay), spectroscopy, ELISA, polyacrylamide gel electrophoresis electropherogram analysis, and chromatographic analysis. See, e.g., Sapan et al. Biotechnol Appl Biochem. 1999. 29(2):99-108. In some embodiments, the concentration of proteins in the RNA composition is 0.8% or less. In some embodiments, the concentration of proteins in the RNA composition is 0.6% or less. In some embodiments, the concentration of proteins in the RNA composition is 0.5% or less. In some embodiments, the concentration of proteins in the RNA composition is 0.4% or less. In some embodiments, the concentration of proteins in the RNA composition is 0.3% or less. In some embodiments, the concentration of proteins in the RNA composition is 0.2% or less.

In some embodiments, the RNA of any of the compositions provided herein is an mRNA.

In some embodiments, at least 50%, at least 60%, at least 70%, at least 75%, at least 80%, at least 85%, or at least 90% of the mRNA molecules of the RNA composition comprise a poly(A) tail. Methods of determining the frequency of mRNAs that comprise poly(A) tails include qRT-PCR-based methods and chromatographic methods. For example, a composition comprising mRNAs may be analyzed by high performance liquid chromatography (HPLC), which will yield a peak corresponding to untailed RNAs, and a peak corresponding to tailed RNAs. The relative heights of the peaks may be compared to determine the relative amount of tailed and untailed RNAs in the composition. In some embodiments, at least 50% of the mRNA molecules comprise a poly(A) tail. In some embodiments, at least 60% of the mRNA molecules comprise a poly(A) tail. In some embodiments, at least 70% of the mRNA molecules comprise a poly(A) tail. In some embodiments, at least 80% of the mRNA molecules comprise a poly(A) tail. In some embodiments, at least 90% of the mRNA molecules comprise a poly(A) tail.

In some embodiments of the compositions provided herein, the RNA is formulated in a lipid nanoparticle. Lipid nanoparticles typically comprise amino lipid, non-cationic lipid, structural lipid, and PEG lipid components along with the nucleic acid cargo of interest. The lipid nanoparticles can be generated using components, compositions, and methods as are generally known in the art, see for example PCT/US2016/052352; PCT/US2016/068300; PCT/US2017/037551; PCT/US2015/027400; PCT/US2016/047406; PCT/US2016/000129; PCT/US2016/014280; PCT/US2017/038426; PCT/US2014/027077; PCT/US2014/055394; PCT/US2016/052117; PCT/US2012/069610; PCT/US2017/027492; PCT/US2016/059575; PCT/US2016/069491; PCT/US2016/069493; and PCT/US2014/66242, all of which are incorporated by reference herein in their entirety.

In some embodiments, the lipid nanoparticle comprises an ionizable amino lipid. In some embodiments, the lipid nanoparticle further comprises: a non-cationic lipid; a sterol; and a polyethylene glycol (PEG)-modified lipid. In some embodiments, the lipid nanoparticle comprises: 40-55 mol % ionizable amino lipid; 5-15 mol % non-cationic lipid; 35-45 mol % sterol; and 1-5 mol % PEG-modified lipid.

In some aspects, the disclosure relates to a composition comprising mRNA formulated in a lipid nanoparticle, wherein a concentration of proteins in the mRNA prior to formulation in the lipid nanoparticle is 0.8% (% w/w) or less, 0.6% or less, 0.4% or less, or 0.2% or less, and wherein at least 50%, at least 60%, at least 70%, at least 75%, at least 80%, at least 85%, or at least 90% of the mRNA molecules of the mRNA composition comprise a poly(A) tail.

Some aspects of the disclosure relate to compositions comprising an IVT mixture (e.g., mRNA, DNA, nucleotide triphosphates, RNA polymerase) and a protease in an amount sufficient to cleave one or more proteins in the mixture into peptide fragments. In some embodiments, the composition comprises an mRNA, a DNA, one or more nucleotide triphosphates, one or more proteins or peptide fragments thereof, and a protease in an amount sufficient to cleave one or more proteins in the mixture into peptide fragments. In some embodiments, one or more peptide fragments in the mixture comprise an amino acid sequence that is present in one or more proteins of the mixture. In some embodiments, the composition comprises one or more proteins, and one or more peptide fragments of the one or more proteins present in the mixture. In some embodiments, at least 80%, at least 85%, at least 90%, at least 95%, at least 96%, at least 97%, at least 98%, at least 99%, or up to 100% of the proteins or peptide fragments in the mixture are 100 kDa or less in size. In some embodiments, at least 90% of the proteins or peptide fragments in the mixture are 100 kDa or less in size. In some embodiments, at least 95% of the proteins and peptide fragments in the mixture are 100 kDa or less in size. In some embodiments, at least 97% of the proteins and peptide fragments in the mixture are 100 kDa or less in size. In some embodiments, at least 99.5% of the proteins and peptide fragments in the mixture are 100 kDa or less in size. In some embodiments, at least 99.75% of the proteins and peptide fragments in the mixture are 100 kDa or less in size. In some embodiments, each of the proteins or peptide fragments in the mixture are 100 kDa or less in size.

In some embodiments, composition comprises a protease selected from the group consisting of proteinase K, Lys-C, trypsin, TPCK-treated trypsin, chymotrypsin, α-lytic protease, and endoproteinase AspN. In some embodiments, the protease is proteinase K. In some embodiments, the proteinase K comprises an amino acid sequence with at least 90%, at least 91%, at least 92%, at least 93%, at least 94%, at least 95%, at least 96%, at least 97%, at least 98%, at least 99%, or up to 100% sequence identity to the amino acid sequence of SEQ ID NO: 3. In some embodiments the proteinase K comprises the amino acid sequence of SEQ ID NO: 3. In some embodiments, the protease is at a concentration of about 0.1 to about 100 Units/mL, about 0.2 to about 50 Units/mL, about 0.3 to about 25 Units/mL, about 0.4 to about 10 Units/mL, about 0.5 to about 5 Units/mL, about 0.5 to about 3 Units/mL, about 0.5 to about 2 Units/mL, or about 0.5 to about 1 Unit/mL. In some embodiments, the concentration of the protease is about 0.1 to about 2 Units/mL. In some embodiments, the composition comprises one or more cations. In some embodiments, the cation is a magnesium ion or a calcium ion. In some embodiments, the concentration of magnesium ions in the mixture is about 10 mM to about 100 mM.

### EXAMPLES

### Example 1: Protease Digestion

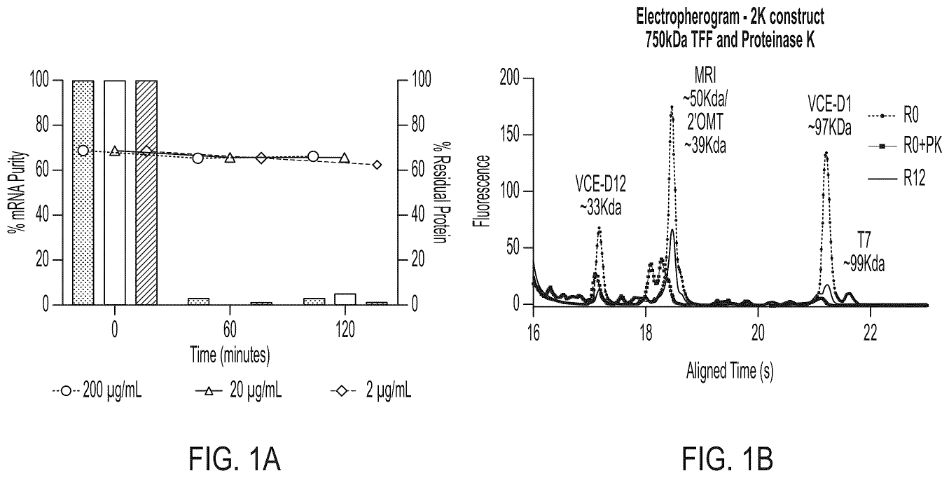

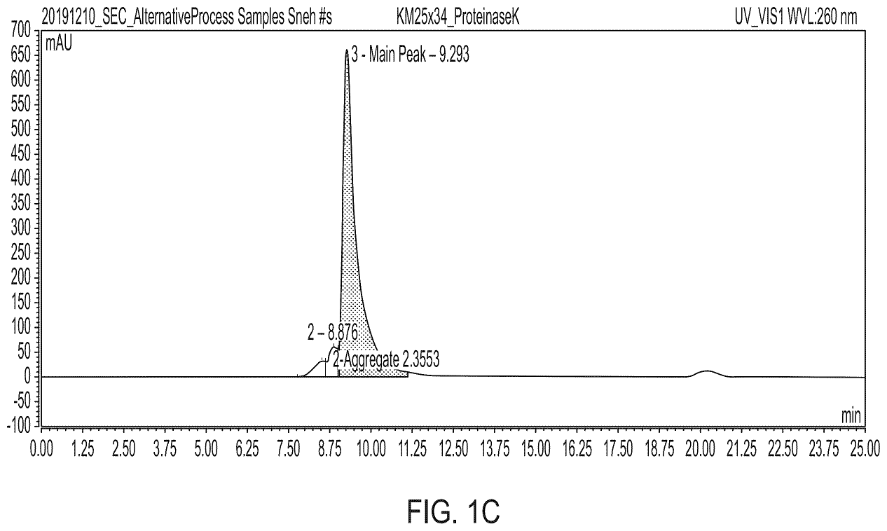

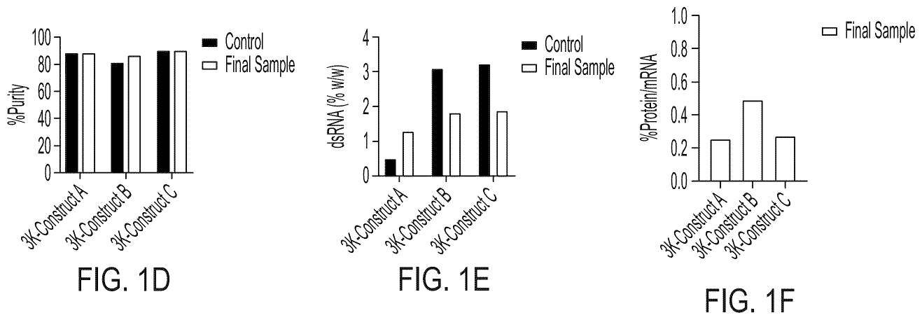

mRNA was in vitro transcribed, and proteinase K was introduced into the in vitro transcription (IVT) mixture at varying concentrations (2 μg/mL, 20 μg/mL, and 200 μg/mL). The purity of mRNA and the percentage of residual protein in the mixture were measured after 60 minutes and 120 minutes of incubation with proteinase K (FIG. 1A). At each concentration, proteinase K degraded nearly all residual protein from the IVT mixture within 60 minutes. Proteinase K digestion followed by tangential flow filtration with a 750 kDa TFF membrane resulted in a marked reduction in the size of electropherogram peaks corresponding to IVT and capping enzymes, including T7 RNA polymerase, MRI, and 2′OMT (FIG. 1B). Alternatively, size exclusion chromatography following proteinase K digestion resulted in the isolation of an mRNA composition that produced a single main peak by HPLC analysis (FIG. 1C). These results indicate that incubation with a protease efficiently digests enzymes used in mRNA transcription and capping, and the resulting peptide fragments are efficiently removed using size-based filtration methods such as tangential flow filtration and size exclusion chromatography.

Three distinct mRNA constructs (A, B, and C) were produced by IVT, and residual protein was removed from the IVT mixture by protease digestion followed by filtration. Purified mRNA compositions were then analyzed for mRNA purity (FIG. 1D), double-stranded RNA (dsRNA) content (FIG. 1E), and residual protein content (FIG. 1F). Compared to control mRNA compositions, protease digestion did not affect mRNA purity (FIG. 1D), and reduced the amount of dsRNA in two of the three compositions (FIG. 1E). Residual protein content was minimal in all samples (FIG. 1F). These results indicate that protease digestion and size-based filtration methods effectively remove residual protein from mRNA compositions, as well as reduce the amount of dsRNA present in a sample.

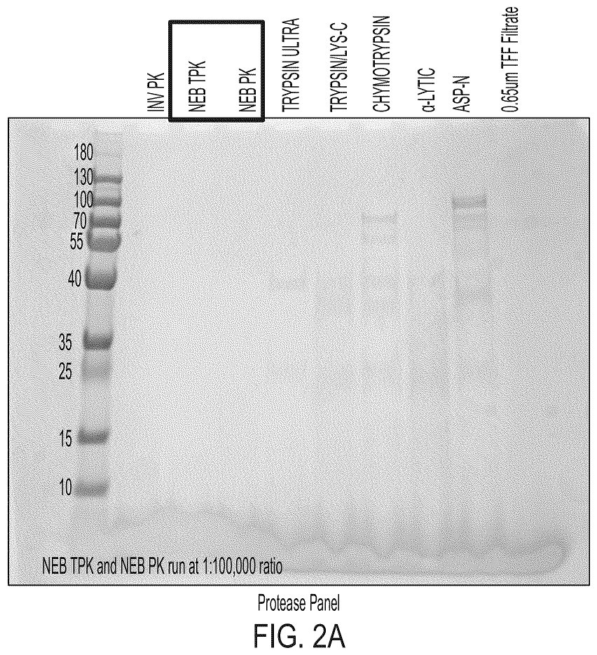

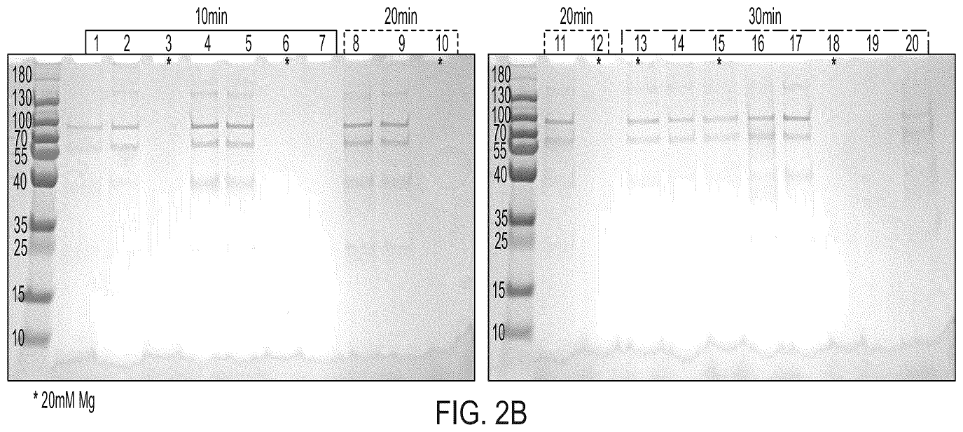

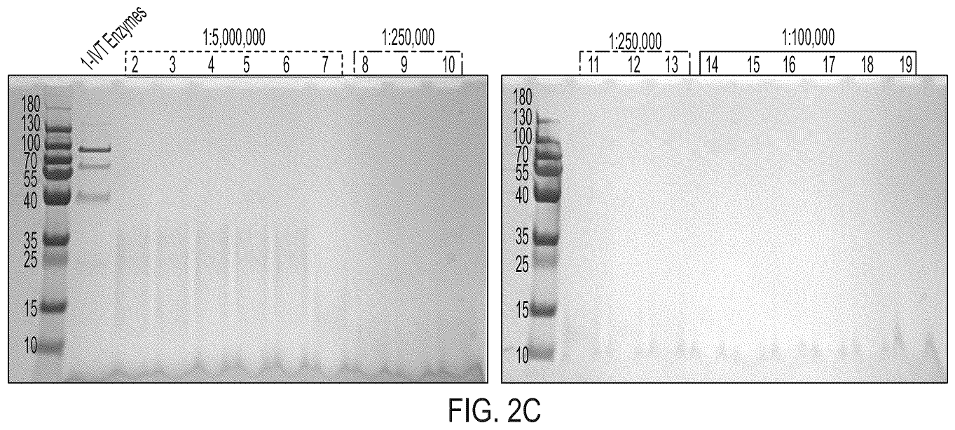

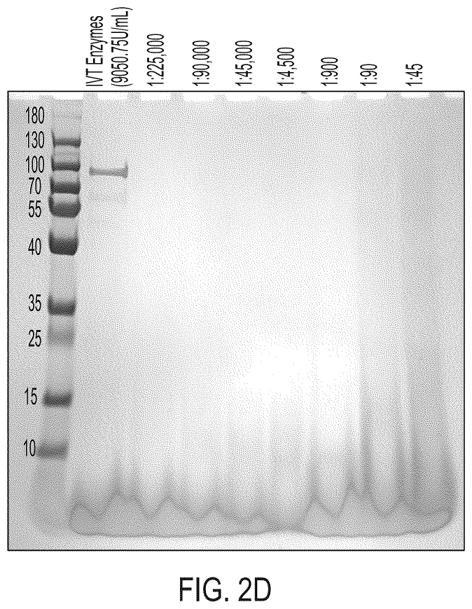

To determine the effectiveness of different proteases in digesting IVT and capping enzymes, multiple proteases were tested in parallel. To identical IVT reactions, one of multiple proteases was added and incubated to allow protease digestion to occur. Following incubation, IVT-digestion reactions were run on SDS-PAGE gel to detect the presence of intact enzymes in each reaction (FIG. 2A). In IVT reactions digested with either thermolabile proteinase K (NEB TPK) and standard proteinase K (INV PK and NEB PK), IVT enzymes were not detectable on a protein gel (FIG. 2A). To further optimize the reaction conditions, proteinase K was added to IVT reactions, and incubated for varying amounts of time. In some reactions, indicated by * above wells, the magnesium was added to the reaction, so that the [Mg²⁺] in the reaction was 20 mM. Ten minutes of incubation were sufficient to digest residual proteins in the IVT reaction, with the presence of Mg²⁺ improving the efficiency of digestion (FIG. 2B). Additionally, different dilutions of proteinase K were tested to evaluate the role of concentration in protease digestion. Proteinase K digestion was most effective at degrading residual proteins at concentrations of 1:250,000 and above (FIG. 2C). Finally, different ratios of proteinase K to IVT enzymes were tested, to determine the amount of proteinase K required to digest a given amount of protein from an IVT reaction. Proteinase K was added to IVT reactions containing varying amounts of IVT enzymes, to a final concentration of 1 Unit/mL proteinase K. When IVT enzymes were diluted at least 900-fold, so that the proteinase K:IVT enzyme ratio was no more than 1:10.5, proteinase K digestion resulted in no detectable protein following a 15 minute incubation (FIG. 2D).

### Example 2: Removal of mRNA During Continuous In Vitro Transcription

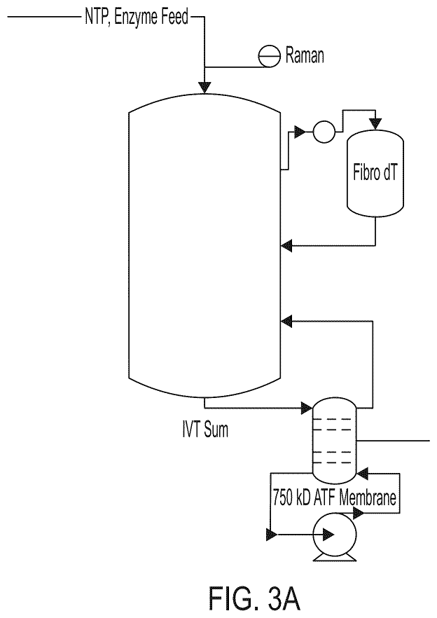

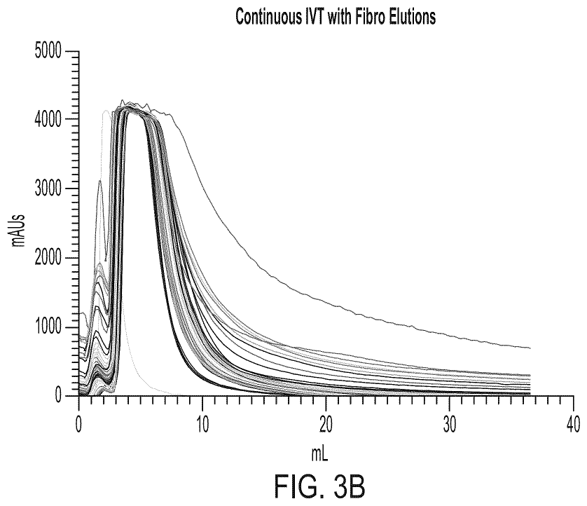

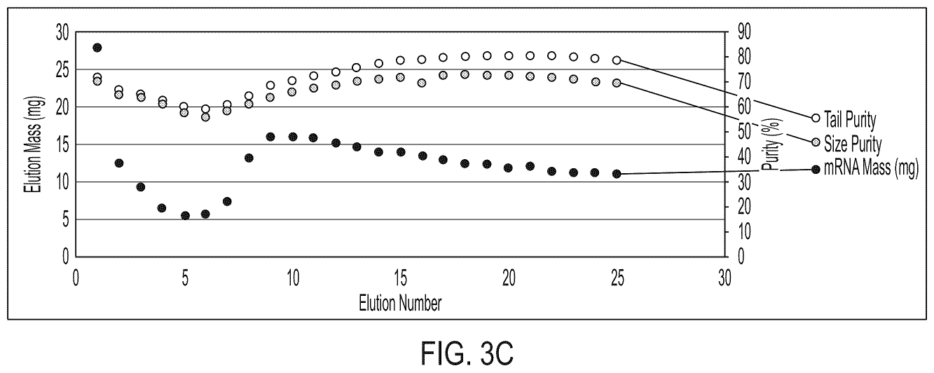

During an in vitro transcription reaction, a sample of the IVT reaction was removed and passed through a hollow fiber membrane coated with oligo-dT to capture mRNA. Flowthrough containing other components of the IVT reaction, including DNA template, IVT enzymes, and nucleotide triphosphates, was returned to the IVT mixture (FIG. 3A). This step of removing mRNA from the IVT mixture was performed continuously, being paused to elute mRNA from oligo-dT-bound, and restarted following elution of mRNA. Each successive elution yielded eluates with consistent main peaks, indicating that mRNA was effectively separated each time (FIG. 3B). With each successive elution, mRNA was consistently pure in terms of percentage of mRNAs that were of the expected size and contained poly(A) tails (FIG. 3C). Feed solution containing NTPs was added during IVT to replenish NTPs consumed in transcription and prevent NTP concentration from becoming limiting. These results indicate that transcribed mRNA can be separated from a continuous IVT reaction, to prevent the mRNA concentration from reaching levels that inhibit transcription, and eluted separately, so that all transcribed mRNA can be collected in a final mRNA composition.

### Example 3: Intensification of dT Chromatography Process

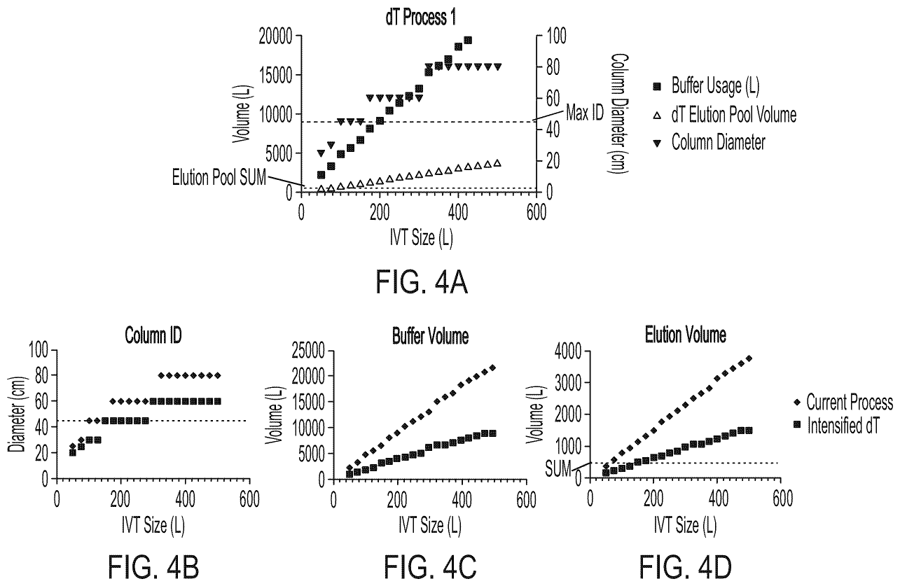

The relationship between the scale of an IVT reaction, and the parameters of an oligo-dT chromatography process for purifying mRNA from the IVT reaction, were analyzed. As an IVT reaction is scaled up, an oligo-dT chromatography process requires increasingly large columns to support binding of additional mRNA and volumes of buffer to support the chromatography (FIG. 4A). Furthermore, mRNA bound to oligo-dT columns must be eluted into larger volumes (FIG. 4A). This need for larger elution volumes quickly becomes rate-limiting, hindering the purification of mRNA from larger scale IVT reactions. However, intensifying the oligo-dT chromatography process by increasing the binding capacity of the oligo-dT columns reduced the required column size (FIG. 4B), buffer volume (FIG. 4C), and elution volume (FIG. 4D). Each of these reductions improved the efficiency of the oligo-dT chromatography process, allowing for IVT reactions to be scaled up without unduly affecting purification of the mRNA.

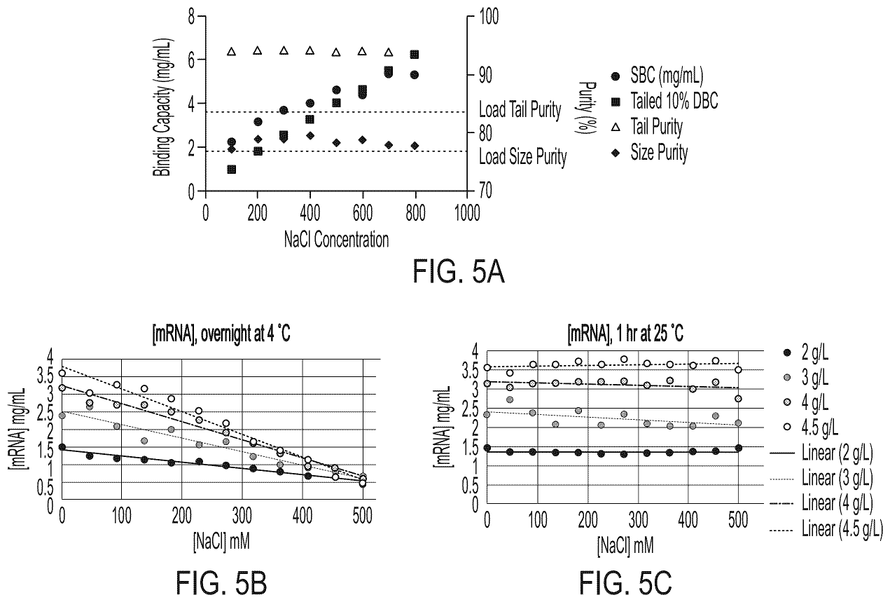

The effects of salt concentration on binding capacities of mRNA to oligo-dT columns and purity of eluted mRNA were analyzed. Increasing salt concentrations improved both the static and dynamic binding capacity of mRNA to oligo-dT columns in a linear manner (FIG. 5A). Surprisingly, this improvement did not affect the percentages of eluted mRNAs that contained poly(A) tails or were of the expected length, indicating that increasing salt concentrations promote binding of mRNA to oligo-dT columns without adversely affecting mRNA purity.

Because increasing the concentration of one solute (e.g., sodium chloride) in a solution may cause other solutes (e.g., nucleic acids) to precipitate, the relationship between salt concentration and mRNA solubility was analyzed. Varying concentrations of sodium chloride were added to solutions containing specified amounts of mRNA, and incubated for 1 hour at 25° C., or overnight at 4° C. Overnight incubation allowed mRNA beyond the threshold limit of 0.5 mg/mL to precipitate from solution (FIG. 5B). However, mRNA precipitation was negligible during brief incubations at room temperature, regardless of salt concentration (FIG. 5C). These results indicate that increased salt concentrations take time to cause the precipitation of mRNA. Therefore, increasing the salt concentration in an mRNA mixture shortly before adding it to an oligo-dT column promotes binding of mRNA to the column without causing unwanted precipitation. Accordingly, increasing salt concentrations are useful for increasing the dynamic binding capacity of a column, thereby improving the efficiency of the oligo-dT chromatography process. As shown in Tables 1-2, the addition of high-salt buffer before adding the mRNA composition to the oligo-dT resin reduces the total processing time or column volume, and consequently oligo-dT resin volume, required to purify mRNA from the composition by 46% to 56%, respectively. In both cases, this improved efficiency increases the productivity of mRNA purification, in terms of (mass)·(volume of stationary phase)⁻¹·(time)⁻¹ (e.g., g·L⁻¹hr⁻¹) Furthermore, salt intensification of oligo-dT chromatography reduced the amount of buffer required to purify the mRNA by 52%-68%.

TABLE 2
Oligo-dT chromatography process 1 with salt intensification.

| | | Process 1 with | Process 1 with |
|---|---|---|---|
| | | 500 mM NaCl | 500 mM NaCl |
| | Process 1 | (same column) | (smaller column) |
| Column diameter | 45 | 45 | 30 |
| (cm) | | | |
| Chromatography | 57.4 | 80.7 | 78.2 |
| cycle duration (min) | | | |
| Number of cycles | 11 | 4 | 10 |
| Chromatography | 11.3 | 6.1 | 13.8 |
| duration (hr) | | | |
| Buffer used (L) | 3125 | 1306 | 1315 |

TABLE 3
Oligo-dT chromatography process 2 with salt intensification.

| | | Process 2 with | Process 2 with |
|---|---|---|---|
| | | 500 mM NaCl | 500 mM NaCl |
| | Process 2 | (same column) | (smaller column) |
| Column diameter | 45 | 45 | 30 |
| (cm) | | | |
| Chromatography | 68.5 | 69.9 | 72.1 |
| cycle duration (min) | | | |
| Number of cycles | 11 | 4 | 10 |
| Chromatography | 12.8 | 6.6 | 12.8 |
| duration (hr) | | | |
| Buffer used (L) | 4119 | 1849 | 1311 |

### Example 4: RNase III Digestion

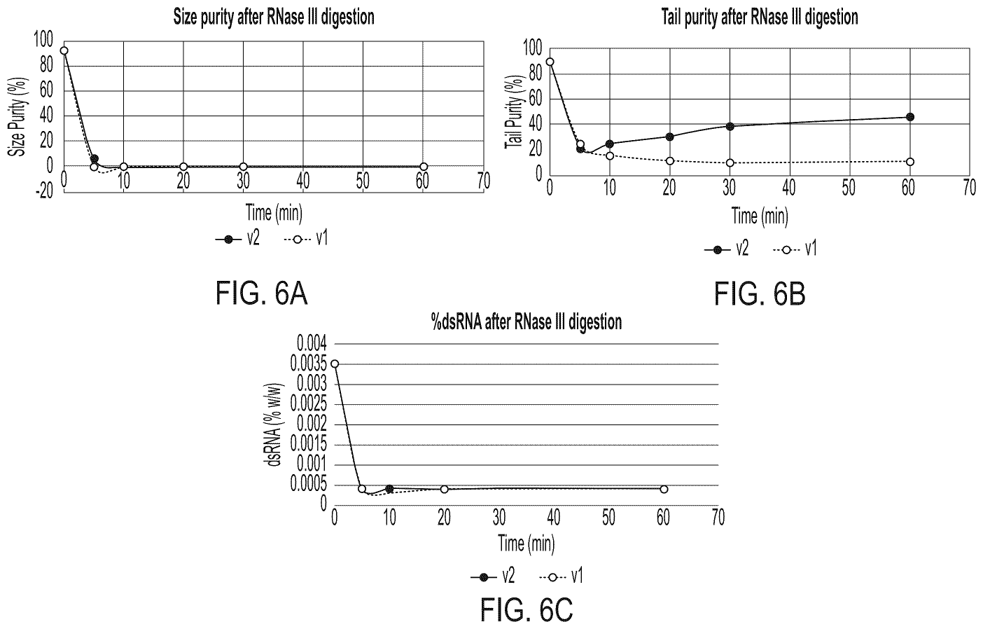

The ability of RNase III to remove dsRNA from an IVT mixture, and the effects of RNase III digestion on mRNA purity, were measured by introducing one of two RNase III variants into mRNA mixtures. mRNA compositions were incubated with either RNase III variant at 37° C., with samples being taken after 5 min, 10 min, 20 min, 30 min, or 60 min to analyze the size purity, tail purity, and dsRNA content of mRNA compositions over the course of RNase III digestion. The contents of each digestion reaction are shown in Table 4, and results are shown in FIGS. 6A-6C.

TABLE 4
RNase III digestion conditions.

| Component | RNase III variant 1 | RNase III variant 2 |
|---|---|---|
| [mRNA] (mg/mL) | 1 | 1 |
| Reaction buffer | 1 | 1 |
| RNase III (U/mL) | 300 | 200 |
| [MnCl₂] (mM) | 0 | 20 |

Both RNase III variants effectively removed dsRNA from mRNA compositions (FIG. 6C), and both digestions degraded mRNA with similar kinetics (FIG. 6A). FIG. 6B shows the degradation of polyA tails of mRNAs following digestion with the RNase III variants. These results indicated that RNase III can remove dsRNA from an mRNA composition with minimal degradation of mRNA.

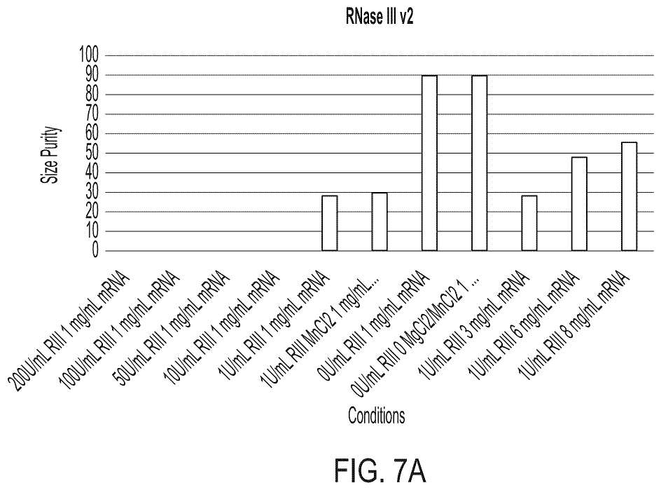

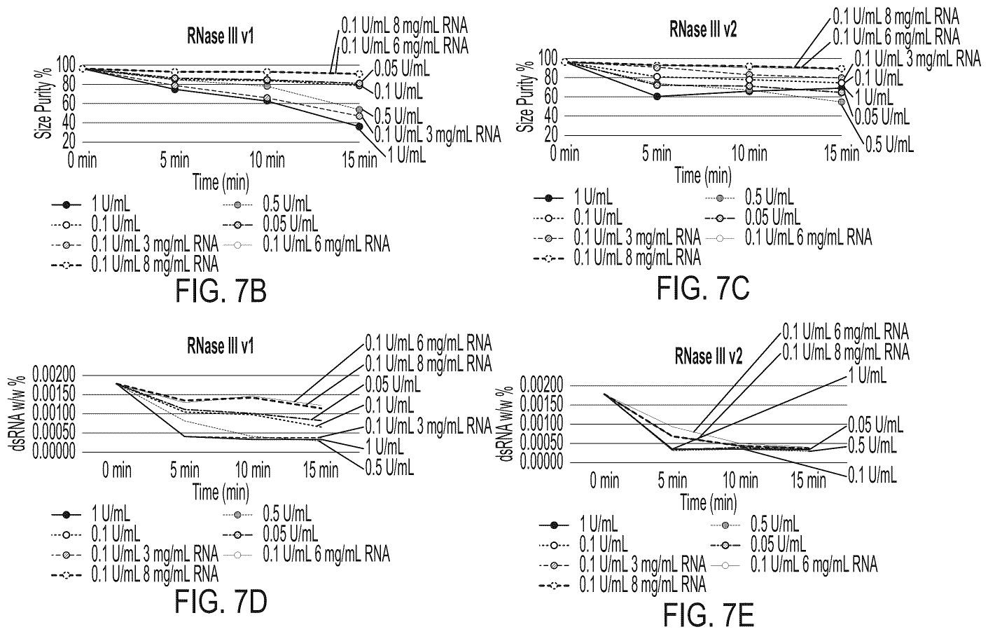

The experiments shown in FIGS. 6A-6C used RNase III at high concentrations sufficient to degrade dsRNA, which had the effect of degrading single-stranded mRNA as well. To determine whether lower concentrations of RNase III could successfully degrade dsRNA without compromising the purity of mRNA, varying concentrations of RNase variant 2 were incubated with mRNAs. Additionally, the effects of increasing mRNA concentration from 1 mg/mL to 8 mg/mL, and of substituting manganese for magnesium, were tested. The results of these experiments are shown in FIG. 7A. At lower concentrations of RNase III, the size purity of mRNAs was maintained, particularly at higher concentrations of mRNA (FIG. 7A). Therefore, the experiments shown in FIGS. 6A-6C were repeated using varying concentrations of each RNase III variant. Compared to RNase III variant 1, RNase III variant 2 more efficiently removed dsRNA from mRNA compositions (FIGS. 7D-7E), without unduly decreasing the size purity of mRNAs (FIGS. 7B-7C).

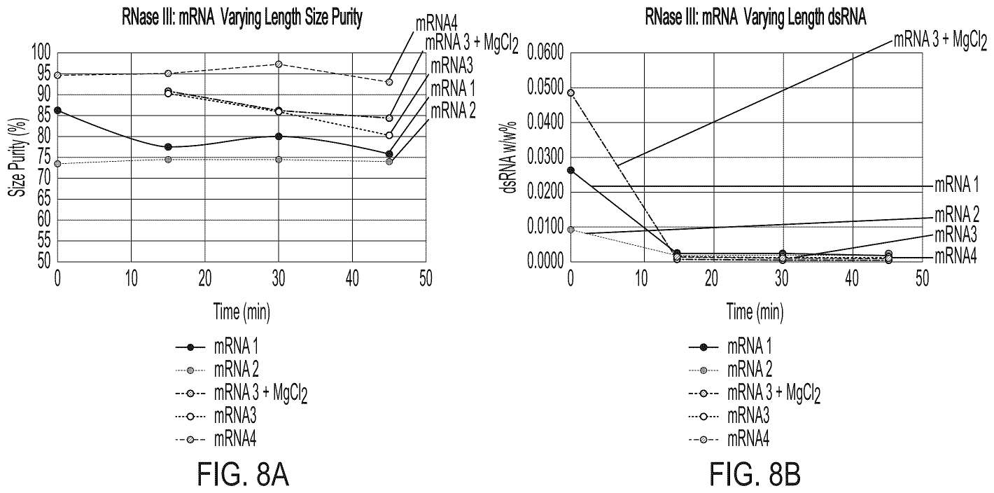

To determine the sensitivity of RNase III digestion efficiency to the molar ratio of RNase III to mRNA, RNase III variant 2 was incubated with mRNAs of varying lengths, but a constant mass concentration of 8 mg/mL, and thus a varying molar concentration, and the kinetics of size purity and dsRNA content were measured. In each digestion, the concentration of RNase III variant 2 concentration was 0.1 U/mL. mRNA size purity was stable over the course of digestion, and was not affected by RNase III presence (FIG. 8A). However, RNase III variant 2 efficiently removed dsRNA from each composition, reducing dsRNA to negligible levels within 15 minutes (FIG. 8B). These results indicate that low concentrations of RNase III variant 2 can effectively remove dsRNA from mRNA compositions without degrading desired mRNA, irrespective of the size of mRNA in the composition.

### EQUIVALENTS AND SCOPE

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in some embodiments, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in some embodiments, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. Each possibility represents a separate embodiment of the present invention.

It should be understood that, unless clearly indicated to the contrary, the disclosure of numerical values and ranges of numerical values in the specification includes both i) the exact value(s) or range specified, and ii) values that are “about” the value(s) or ranges specified (e.g., values or ranges falling within a reasonable range (e.g., about 10% similar)) as would be understood by a person of ordinary skill in the art.

It should also be understood that, unless clearly indicated to the contrary, in any methods disclosed herein that include more than one step or act, the order of the steps or acts of the method is not necessarily limited to the order in which the steps or acts of the method are disclosed.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method of purifying in vitro transcribed mRNA, the method comprising:
(a) adding a high-salt buffer to a composition comprising mRNA to produce a high-salt mRNA composition comprising a salt concentration of at least 100 mM;
(b) contacting a stationary phase with the composition produced in (a); and
(c) eluting mRNA from the stationary phase of (b) to obtain eluted mRNA.

2. The method of claim 1, wherein the stationary phase of (b) comprises fiber, particles, resin, beads, a membrane, and/or monolithic stationary phase.

3. The method of claim 1 or 2, wherein the stationary phase of (b) comprises an oligonucleotide comprising a nucleic acid sequence that is complementary to a nucleotide sequence of the mRNA.

4. The method of any one of claims 1-3, wherein the stationary phase of (b) comprises oligo-dT resin.

5. The method of any one of claims 1-4, wherein the stationary phase of (b) comprises a hydrophobic interaction chromatography (HIC) ligand, optionally wherein the HIC ligand comprises a butyl, phenyl, octyl, t-butyl, methyl, and/or ethyl functional group.

6. The method of any one of claims 1-5, wherein the high-salt mRNA composition has a salt concentration of at least 200 mM, at least 300 mM, at least 400 mM, at least 500 mM, at least 600 mM, at least 700 mM, at least 800 mM, at least 900 mM, at least 1 M, or more.

7. The method of any one of claims 1-6, wherein the high-salt mRNA composition has a salt concentration of about 400 mM to about 600 mM, optionally wherein the high-salt mRNA composition has a salt concentration of about 500 mM.

8. The method of any one of claims 1-7, wherein the salt concentration of the high-salt mRNA composition is the concentration of sodium chloride, potassium chloride, ammonium chloride, ammonium sulfate, monosodium phosphate, disodium phosphate, or trisodium phosphate in the composition.

9. The method of any one of claims 1-8, wherein the high-salt mRNA composition comprises a sodium chloride concentration of about 400 mM to about 600 mM, optionally wherein the composition has a sodium chloride concentration of about 500 mM.

10. The method of any one of claims 1-9, wherein the contacting of (b) occurs within 1 hour or less, 30 minutes or less, 25 minutes or less, 20 minutes or less, 15 minutes or less, 10 minutes or less, 5 minutes or less, 4 minutes or less, 3 minutes or less, 2 minutes or less, or 1 minute or less, of the adding of the high-salt buffer to the composition comprising mRNA of (a).

11. The method of any one of claims 1-10, wherein the high-salt buffer is added by in-line mixing.

12. The method of any one of claims 1-10, wherein the high-salt buffer is added by bolus addition.

13. The method of any one of claims 1-12, further comprising desalting the composition comprising mRNA before adding the high-salt buffer of (a) to produce a desalted mRNA composition with a salt concentration of less than 20 mM.

14. The method of claim 13, wherein the desalting comprises binding the mRNA composition to a hydrophobic interaction chromatography (HIC) resin and eluting the mRNA from the HIC resin to produce a desalted mRNA composition.

15. The method of any one of claims 1-14, wherein the high-salt mRNA composition comprises at least 2.0 g/L, 2.5 g/L, 3.0 g/L, 3.5 g/L, 4.0 g/L, 4.5 g/L, 5.0 g/L, 6.0 g/L, 7.0 g/L, 8.0 g/L, 9.0 g/L, 10.0 g/L, or more dissolved mRNA.

16. The method of any one of claims 1-15, wherein the high-salt mRNA composition of (a) comprises about 4.0 g/L to about 6.0 g/L dissolved mRNA.

17. The method of any one of claims 1-16, wherein at least 80%, at least 85%, at least 90%, at least 95%, at least 97%, at least 98%, at least 99%, or up to 100% of mRNAs in the high-salt mRNA composition are dissolved mRNAs.

18. The method of any one of claims 1-17, wherein the stationary phase of (b) is comprised in a column, wherein the concentration of mRNA in the composition of (b) is 100% or less, 90% or less, or 80% or less of the dynamic binding capacity of the column.

19. The method of any one of claims 1-18, wherein the mRNA is produced by an in vitro transcription step comprising:
in a reaction vessel comprising a mixture comprising a DNA molecule, nucleotide triphosphates (NTPs) including adenosine triphosphate (ATP), cytidine triphosphate (CTP), uridine triphosphate (UTP), and guanosine triphosphate (GTP), and an RNA polymerase, in vitro transcribing a DNA molecule, whereby the RNA polymerase transcribes the DNA molecule to produce an in vitro transcribed mRNA.

20. The method of claim 19, further comprising:
(i) contacting the mixture with a protease;
(ii) incubating the mixture for a period of time sufficient for the protease to cleave one or more proteins in the mixture to produce peptide fragments; and
(iii) isolating the mRNA from the mixture to obtain an isolated mRNA composition.

21. A method of purifying in vitro transcribed mRNA, the method comprising:
(i) contacting a mixture comprising the mRNA with a protease;
(ii) incubating the mixture for a period of time sufficient for the protease to cleave one or more proteins in the mixture to produce peptide fragments; and
(iii) isolating the mRNA from the mixture to obtain an isolated mRNA composition.

22. The method of claim 20 or 21, wherein the protease is selected from the group consisting of proteinase K, Lys-C, trypsin, TPCK-treated trypsin, chymotrypsin, α-lytic protease, and endoproteinase AspN.

23. The method of any one of claims 20-22, wherein the protease is proteinase K.

24. The method of claim 23, wherein the proteinase K comprises an amino acid sequence with at least 90%, at least 91%, at least 92%, at least 93%, at least 94%, at least 95%, at least 96%, at least 97%, at least 98%, at least 99%, or up to 100% sequence identity to the amino acid sequence of SEQ ID NO: 3.

25. The method of claim 23 or 24, wherein the proteinase K comprises the amino acid sequence of SEQ ID NO: 3.

26. The method of any one of claims 20-25, wherein the protease is at a concentration of about 0.1 to about 100 Units/mL, about 0.2 to about 50 Units/mL, about 0.3 to about 25 Units/mL, about 0.4 to about 10 Units/mL, about 0.5 to about 5 Units/mL, about 0.5 to about 3 Units/mL, about 0.5 to about 2 Units/mL, or about 0.5 to about 1 Unit/mL.

27. The method of any one of claims 20-26, wherein the concentration of the protease is about 0.1 to about 2 Units/mL.

28. The method of any one of claims 20-27, wherein the protease:protein concentration in the mixture is about 1:10 to about 1:100, about 1:100 to about 1:1,000, about 1:1,000 to about 1:10,000, about 1:10,000 to about 1:100,000, or about 1:100,000 to about 1:1,000,000.

29. The method of any one of claims 20-28, wherein the protease:protein concentration in the mixture is about 1:1,000 to about 1:50,000.

30. The method of any one of claims 20-29, wherein the mixture of (i) comprises one or more cations.

31. The method of any one of claims 20-30, wherein step (i) and/or step (ii) comprises adding one or more cations to the mixture.

32. The method of claim 30 or 31, wherein the cation is a magnesium ion or a calcium ion.

33. The method of claim 32, wherein the concentration of magnesium ions in the mixture during step (ii) is about 10 mM to about 100 mM.

34. The method of any one of claims 20-33, wherein the incubating of step (ii) is conducted at about 37° C.

35. The method of any one of claims 20-34, wherein the incubating of step (ii) is conducted for about 10 minutes to about 6 hours.

36. The method of any one of claims 20-35, wherein the isolating of step (iii) comprises separating the mRNA from the protease and peptide fragments by tangential flow filtration (TFF).

37. The method of claim 36, wherein the tangential flow filtration comprises using a TFF membrane with a molecular weight cutoff of 500 kDa or less, 200 kDa or less, or 100 kDa or less.

38. The method of claim 36 or 37, wherein the tangential flow filtration comprises using a TFF membrane with a molecular weight cutoff of 100 kDa or less.

39. The method of any one of claims 20-38, wherein the contacting of step (i) further comprises contacting the mixture comprising the mRNA with a DNase, and wherein the incubating of step (ii) further comprises incubating the mixture for a period of time sufficient for the DNase to cleave one or more DNAs in the mixture to produce DNA fragments.

40. The method of any one of claims 20-39, wherein the in vitro transcribed mRNA is produced by a method comprising the steps of:
(i) in a reaction vessel comprising a mixture comprising a DNA molecule, nucleotide triphosphates (NTPs) including adenosine triphosphate (ATP), cytidine triphosphate (CTP), uridine triphosphate (UTP), and guanosine triphosphate (GTP), and an RNA polymerase, in vitro transcribing a DNA molecule, whereby the RNA polymerase transcribes the DNA molecule to produce an mRNA, wherein mRNA is removed from the reaction vessel by the steps of:
(1) transferring a portion of the mixture from the reaction vessel to a column comprising a stationary phase;
(2) passing the portion of the mixture through the column, whereby the stationary phase retains mRNA from the mixture; and
(3) re-introducing a flowthrough from the column into the reaction vessel, wherein the concentration of mRNA in the flowthrough of step (3) is lower than the concentration of mRNA in the portion of the mixture of step (1).

41. A method of removing in vitro transcribed mRNA from an in vitro transcription reaction, the method comprising:
(i) in a reaction vessel comprising a mixture comprising a DNA molecule, nucleotide triphosphates (NTPs) including adenosine triphosphate (ATP), cytidine triphosphate (CTP), uridine triphosphate (UTP), and guanosine triphosphate (GTP), and an RNA polymerase, in vitro transcribing a DNA molecule, whereby the RNA polymerase transcribes the DNA molecule to produce an mRNA, wherein the mRNA is removed from the reaction vessel by the steps of:
(1) transferring a portion of the mixture from the reaction vessel to a column comprising a stationary phase;
(2) passing the portion of the mixture through the column, whereby the stationary phase retains mRNA from the mixture; and
(3) re-introducing a flowthrough from the column into the reaction vessel, wherein the concentration of mRNA in the flowthrough of step (3) is lower than the concentration of mRNA in the portion of the mixture of step (1); and
(ii) isolating the mRNA from the mixture to obtain an isolated mRNA composition.

42. The method of claim 41, wherein the method further comprises, prior to the isolation of step (ii), contacting the mixture with a DNase, and incubating the mixture for a period of time sufficient for the DNase to cleave one or more DNAs in the mixture to produce DNA fragments.

43. The method of any one of claims 40-42, wherein the stationary phase comprises fiber, particles, resin, and/or beads.

44. The method of claim 43, wherein the stationary phase comprises oligo-dT.

45. The method of claim 44, wherein the stationary phase comprises oligo-dT fiber.

46. The method of any one of claims 40-45, wherein the concentration of NTPs in the reaction vessel is between about 30 mM and about 50 mM.

47. The method of any one of claims 40-46, wherein:
(a) the concentration of GTP in the reaction mixture is at least 2× the concentration of each of ATP, CTP, and UTP;
(b) the reaction mixture further comprises guanosine diphosphate (GDP), and wherein the concentration of GDP is at least 2× the concentration of each of ATP, CTP, and UTP; and/or
(c) the reaction mixture further comprises GDP, and wherein the ratio of concentration of GTP plus GDP to the concentration of each of ATP, CTP, and UTP is at least 2:1.

48. The method of claim 47, wherein the ratio of concentrations of GTP:ATP:CTP:UTP is 4:2:1:1, 4:2:2:1, or 6:3:3:1.

49. The method of any one of claims 40-48, wherein the in vitro transcribing step of (i) further comprises adding a feed solution comprising GTP, ATP, CTP, and UTP.

50. The method of claim 49, wherein:
(a) 25-35% of NTPs in the feed solution are GTP;
(b) 20-30% of NTPs in the feed solution are ATP;
(c) 30-40% of NTPs in the feed solution are CTP; and/or
(d) 10-20% of NTPs in the feed solution are UTP.

51. The method of claim 49 or 50, wherein, after addition of the feed solution, the concentration of GTP in the reaction mixture is at least 2× the concentration of each of ATP, CTP, and UTP.

52. The method of any one of claims 49-51, wherein the feed solution further comprises GDP, wherein, after addition of the feed solution:
(a) the concentration of GDP is at least 2× the concentration of each of ATP, CTP, and UTP; and/or
(b) the ratio of concentration of GTP plus GDP to the concentration of each of ATP, CTP, and UTP is at least 2:1.

53. The method of any one of claims 49-52, wherein, after addition of the feed solution:
(a) the ratio of GTP:ATP is in the reaction mixture is 1.5:1 to 2.5:1,
the ratio of GTP:CTP is in the reaction mixture is 3.5:1 to 4.5:1, and
the ratio of GTP:UTP is in the reaction mixture is 3.5:1 to 4.5:1;
(b) the ratio of GTP:ATP is in the reaction mixture is 1.5:1 to 2.5:1,
the ratio of GTP:CTP is in the reaction mixture is 1.5:1 to 2.5:1, and
the ratio of GTP:UTP is in the reaction mixture is 3.5:1 to 4.5:1; or
(c) the ratio of GTP:ATP is in the reaction mixture is 1.5:1 to 2.5:1,
the ratio of GTP:CTP is in the reaction mixture is 1.5:1 to 2.5:1, and
the ratio of GTP:UTP is in the reaction mixture is 5.5:1 to 6.5:1

54. The method of claim 53, wherein, after addition of the feed solution, the ratio of concentrations of GTP:ATP:CTP:UTP in the reaction mixture is 4:2:1:1, 4:2:2:1, or 6:3:3:1.

55. The method of claim 49 or 50, wherein the feed solution further comprises magnesium ions.

56. The method of claim 55, wherein the concentration of magnesium ions in the reaction vessel, after addition of the feed solution, is between 200 mM and 500 mM.

57. The method of any one of claims 49-55, wherein the feed solution is added to the reaction vessel continuously.

58. The method of any one of claims 49-56, wherein the feed solution is added to the reaction vessel as a bolus.

59. The method of any one of claims 40-58, further comprising reducing the volume of the reaction mixture.

60. The method of claim 59, wherein reducing the volume of the reaction mixture comprises tangential flow filtration (TFF).

61. The method of claim 60, wherein the tangential flow filtration comprises using a TFF membrane with a molecular weight cutoff of 50 kDa or less.

62. The method of any one of claims 40-61, wherein the concentration of mRNA in the reaction vessel is maintained at a concentration below 20 mg/mL, below 15 mg/mL, below 12 mg/mL, or below 10 mg/mL.

63. The method of any one of claims 40-61, wherein the concentration of mRNA in the reaction vessel is maintained at a concentration of 8 mg/mL or more, 9 mg/mL or more, 10 mg/mL or more, or 11 mg/mL or more.

64. The method of any one of claims 40-63, further comprising eluting mRNA from the column to collect an eluate comprising mRNA.

65. The method of claim 64, wherein the eluting is performed more than once.

66. The method of claim 64 or 65, wherein the steps of (1), (2), and (3) are repeated after the eluting step.

67. The method of claim 66, wherein the steps of (1), (2), and (3) are performed continuously, paused before elution, restarted after elution, and performed continuously after elution.

68. The method of any one of claims 64-67, wherein the eluate is added to the mixture prior to the isolating of step (ii).

69. The method of any one of claims 1-68, further comprising:
(a) contacting the mixture with an RNase III;
(b) incubating the RNase III to cleave one or more double-stranded RNAs (dsRNAs) in the mixture; and
(c) isolating mRNA from the mixture to obtain an isolated mRNA composition.

70. A method of reducing double-stranded RNA in an mRNA composition, the method comprising:
(i) in a reaction vessel comprising a mixture comprising a DNA molecule and an RNA polymerase, in vitro transcribing a DNA molecule, whereby the RNA polymerase transcribes the DNA molecule to produce an mRNA;
(ii) contacting the mixture with an RNase III;
(iii) incubating the RNase III to cleave one or more double-stranded RNAs (dsRNAs) in the mixture; and
(iv) isolating the mRNA from the mixture to obtain an isolated mRNA composition.

71. The method of claim 70, wherein the RNase III is present in the mixture during the in vitro transcribing step, wherein the step of incubating the RNase III is conducted during the in vitro transcribing step.

72. The method of claim 71, wherein the RNase III is added to the in vitro transcription mixture after 30 minutes, 60 minutes, 90 minutes, 100 minutes, 110 minutes, 120 minutes, 130 minutes, 140 minutes, 150 minutes, 160 minutes, or 170 minutes of in vitro transcription.

73. The method of any one of claims 69-72, wherein the RNase III comprises an amino acid sequence with at least 80% sequence identity to the amino acid sequence of SEQ ID NO: 4, wherein the RNase III comprises an amino acid substitution corresponding to an E38A substitution in SEQ ID NO: 4.

74. The method of claim 73, wherein the RNase III comprises the amino acid sequence of SEQ ID NO: 5.

75. The method of any one of claims 69-74, wherein the mixture comprises magnesium ions during the step of incubating the RNase III.

76. The method of claim 75, wherein the concentration of magnesium ions in the mixture during the step of incubating the RNase III is between about 10 mM to about 100 mM, optionally wherein the concentration of magnesium ions is about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, or about 30 mM.

77. The method of any one of claims 69-76, wherein the concentration of RNase III in the mixture during the step of incubating the RNase III is less than 0.2 U/mL, less than 0.15 U/mL, less than 0.1 U/mL, less than 0.09 U/mL, less than 0.08 U/mL, less than 0.07 U/mL, less than 0.06 U/mL, or less than 0.05 U/mL.

78. The method of any one of claims 69-76, wherein the RNase III is incubated for a period of time sufficient to cleave at least 80%, at least 90%, at least 95%, at least 97%, at least 98%, at least 99%, or up to 100% of dsRNAs in the mixture.

79. The method of any one of claims 69-78, wherein the RNase III is incubated for about 1 minute to about 5 minutes, about 5 minutes to about 10 minutes, about 10 minutes to about 20 minutes, about 20 minutes to about 30 minutes, about 30 minutes to about 40 minutes, about 40 minutes to about 50 minutes, or about 50 minutes to about 60 minutes.

80. An isolated mRNA composition comprising mRNA produced by the method of any one of claims 1-79.

81. The composition of claim 80, wherein the concentration of proteins in the isolated mRNA composition is 0.8% (% w/w) or less, 0.6% or less, 0.4% or less, or 0.2% or less.

82. The composition of claim 80 or 81, wherein the concentration of double-stranded RNA (dsRNA) in the isolated mRNA composition is 0.05% (% w/w) or less, 0.04% or less, 0.03% or less, 0.02% or less, 0.01% or less, or 0.008% or less, 0.006% or less, 0.004% or less, 0.002% or less, or 0.001% or less.

83. The composition of any one of claims 79-82, wherein at least 50%, at least 60%, at least 70%, at least 75%, at least 80%, at least 85%, or at least 90% of the mRNA molecules of the isolated mRNA composition comprise a poly(A) tail.

84. The composition of any one of claims 79-83, wherein the mRNA is formulated in a lipid nanoparticle.

85. The composition of claim 84, wherein the lipid nanoparticle comprises: an ionizable amino lipid.

86. The composition of claim 85, wherein the lipid nanoparticle further comprises: a non-cationic lipid; a sterol; and a polyethylene glycol (PEG)-modified lipid.

87. The composition of claim 86, wherein the lipid nanoparticle comprises: 40-55 mol % ionizable amino lipid; 5-15 mol % non-cationic lipid; 35-45 mol % sterol; and 1-5 mol % PEG-modified lipid.

88. A composition comprising mRNA formulated in a lipid nanoparticle, wherein a concentration of proteins in the mRNA prior to formulation in the lipid nanoparticle is 0.8% (% w/w) or less, 0.6% or less, 0.4% or less, or 0.2% or less, and wherein at least 50%, at least 60%, at least 70%, at least 75%, at least 80%, at least 85%, or at least 90% of the mRNA molecules of the mRNA composition comprise a poly(A) tail.

89. A composition comprising:
(i) an mRNA;
(ii) a DNA;
(iii) one or more nucleotide triphosphates;
(iv) one or more proteins or peptide fragments thereof;
(v) a protease in an amount sufficient to cleave one or more proteins in the mixture into peptide fragments.

90. The composition of claim 89, wherein the composition comprises one or more proteins and one or more peptide fragments thereof.

91. The composition of claim 89 or 90, wherein at least 80%, at least 85%, at least 90%, at least 95%, at least 96%, at least 97%, at least 98%, at least 99%, or up to 100% of the proteins or peptide fragments in the mixture are 100 kDa or less in size.

92. The composition of any one of claims 89-91, wherein the protease is selected from the group consisting of proteinase K, Lys-C, trypsin, TPCK-treated trypsin, chymotrypsin, α-lytic protease, and endoproteinase AspN.

93. The composition of any one of claim 89-92, wherein the protease is proteinase K.

94. The composition of claim 93, wherein the proteinase K comprises an amino acid sequence with at least 90%, at least 91%, at least 92%, at least 93%, at least 94%, at least 95%, at least 96%, at least 97%, at least 98%, at least 99%, or up to 100% sequence identity to the amino acid sequence of SEQ ID NO: 3.

95. The composition of claim 93 or 94, wherein the proteinase K comprises the amino acid sequence of SEQ ID NO: 3.

96. The composition of any one of claims 89-95, wherein the protease is at a concentration of about 0.1 to about 100 Units/mL, about 0.2 to about 50 Units/mL, about 0.3 to about 25 Units/mL, about 0.4 to about 10 Units/mL, about 0.5 to about 5 Units/mL, about 0.5 to about 3 Units/mL, about 0.5 to about 2 Units/mL, or about 0.5 to about 1 Unit/mL.

97. The composition of any one of claims 89-96, wherein the concentration of the protease is about 0.1 to about 2 Units/mL.

98. The composition of any one of claims 89-97, wherein the composition comprises one or more cations.

99. The composition of claim 98, wherein the cation is a magnesium ion or a calcium ion.

100. The composition of claim 99, wherein the concentration of magnesium ions in the mixture is about 10 mM to about 100 mM.


SEQUENCE LISTING (9 sequences)

SEQ ID NO: 1 - protein, 884 aa, Artificial Sequence
  Synthetic
MNTINIAKNDFSDIELAAIPFNTLADHYGERLAREQLALEHESYEMAEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVWVIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFAG

SEQ ID NO: 2 - DNA, 1420 bp, Unknown
  Parengyodontium album (Tritirachium album)
aatcgcagcaatacaaagcatcctctcaaactcaacaaactttcttgattaagcaccgctttctcattcatcaacaacagccaaaatgcgtttgtctgttcttctgagtcttcttcccctcgctctcggcgctcctgccgttgagcagcgctccgaggctgctcctctgatcgaggcccgcggcgagatggttgccaacaagtacattgtcaagttcaaggagggtagcgctctttctgctctcgatgctgccatggagaagatttctggcaagcccgaccacgtctacaagaacgtcttcagtggtttcgctgcgacccttgacgagaacatggttcgggttctccgcgcccatcccgatgttgagtacattgagcaggatgctgttgtcaccatcaacgctgcgcagaccaacgctccctggggccttgctcgcatctccagcaccagccccggtacctctacttactactatgacgaatctgccggccaaggctcctgcgtctacgtgattgacaccggtatcgaggcatcgcaccccgagtttgagggtcgtgcccagatggtcaagacctactactactccagtcgcgacggtaacggtcacggcactcactgcgctggtaccgttggctcccgaacctacggtgtcgccaagaagacccagctctttggtgtcaaggtcctcgatgacaacggcagtggccagtactccaccatcatcgccggtatggactttgttgccagcgacaagaacaaccgcaactgccccaaaggtgtcgttgcctccttgtcccttggcggtggttactcctcctccgtgaacagcgccgctgccaggctccagagctctggtgtcatggtcgccgtcgctgccggtaacaacaacgctgacgcccgcaactactcccctgcttctgagccctcggtctgcactgtcggtgcttctgaccgctacgacagacgctccagcttctccaactacggcagcgttttggacatctttggccctggtaccagcattctctccacctggatcggcggcagcacccgctccatctctggaacttccatggctactccccacgttgccggtctcgctgcctacctcatgactcttggaaagactaccgccgccagcgcttgccgatacattgccgacaccgccaacaagggcgacttgagcaacattcccttcggcactgtcaacctgcttgcctacaacaactaccaggcttaagtccttcagtcaacgccaaaagctcaagatatgaaacaagttatgaatgcatctgtatatagacgagcatacttcatatcagtacataaccatccttagtatatttaaaaaggcaacgaatttgcagatttagctgtcagacagttagatttgcatgaatcagaagatatttgctttcat

SEQ ID NO: 3 - protein, 384 aa, Unknown
  Parengyodontium album (Tritirachium album)
MRLSVLLSLLPLALGAPAVEQRSEAAPLIEARGEMVANKYIVKFKEGSALSALDAAMEKISGKPDHVYKNVFSGFAATLDENMVRVLRAHPDVEYIEQDAVVTINAAQTNAPWGLARISSTSPGTSTYYYDESAGQGSCVYVIDTGIEASHPEFEGRAQMVKTYYYSSRDGNGHGTHCAGTVGSRTYGVAKKTQLFGVKVLDDNGSGQYSTIIAGMDFVASDKNNRNCPKGVVASLSLGGGYSSSVNSAAARLQSSGVMVAVAAGNNNADARNYSPASEPSVCTVGASDRYDRRSSFSNYGSVLDIFGPGTSILSTWIGGSTRSISGTSMATPHVAGLAAYLMTLGKTTAASACRYIADTANKGDLSNIPFGTVNLLAYNNYQA

SEQ ID NO: 4 - protein, 226 aa, Escherichia coli
MNPIVINRLQRKLGYTFNHQELLQQALTHRSASSKHNERLEFLGDSILSYVIANALYHRFPRVDEGDMSRMRATLVRGNTLAELAREFELGECLRLGPGELKSGGFRRESILADTVEALIGGVFLDSDIQTVEKLILNWYQTRLDEISPGDKQKDPKTRLQEYLQGRHLPLPTYLVVQVRGEAHDQEFTIHCQVSGLSEPVVGTGSSRRKAEQAAAEQALKKLELE

SEQ ID NO: 5 - protein, 226 aa, Artificial Sequence
  Synthetic
MNPIVINRLQRKLGYTFNHQELLQQALTHRSASSKHNARLEFLGDSILSYVIANALYHRFPRVDEGDMSRMRATLVRGNTLAELAREFELGECLRLGPGELKSGGFRRESILADTVEALIGGVFLDSDIQTVEKLILNWYQTRLDEISPGDKQKDPKTRLQEYLQGRHLPLPTYLVVQVRGEAHDQEFTIHCQVSGLSEPVVGTGSSRRKAEQAAAEQALKKLELE

SEQ ID NO: 6 - protein, 240 aa, Thermotoga maritima
MNESERKIVEEFQKETGINFKNEELLFRALCHSSYANEQNQAGRKDVESNEKLEFLGDAVLELFVCEILYKKYPEAEVGDLARVKSAAASEEVLAMVSRKMNLGKFLFLGKGEEKTGGRDRDSILADAFEALLAAIYLDQGYEKIKELFEQEFEFYIEKIMKGEMLFDYKTALQEIVQSEHKVPPEYILVRTEKNDGDRIFVVEVRVNGKTIATGKGRTKKEAEKEAARIAYEKLLKERS

SEQ ID NO: 7 - protein, 240 aa, Artificial Sequence
  Synthetic
MNESERKIVEEFQKETGINFKNEELLFRALCHSSYANEQNQAGRKDVESNAKLEFLGDAVLELFVCEILYKKYPEAEVGDLARVKSAAASEEVLAMVSRKMNLGKFLFLGKGEEKTGGRDRDSILADAFEALLAAIYLDQGYEKIKELFEQEFEFYIEKIMKGEMLFDYKTALQEIVQSEHKVPPEYILVRTEKNDGDRIFVVEVRVNGKTIATGKGRTKKEAEKEAARIAYEKLLKERS

SEQ ID NO: 8 - protein, 221 aa, Aquifex aeolicus
MKMLEQLEKKLGYTFKDKSLLEKALTHVSYSKKEHYETLEFLGDALVNFFIVDLLVQYSPNKREGFLSPLKAYLISEEFFNLLAQKLELHKFIRIKRGKINETIIGDVFEALWAAVYIDSGRDANFTRELFYKLFKEDILSAIKEGRVKKDYKTILQEITQKRWKERPEYRLISVEGPHHKKKFIVEAKIKEYRTLGEGKSKKEAEQRAAEELIKLLEESE

SEQ ID NO: 9 - protein, 221 aa, Artificial Sequence
  Synthetic
MKMLEQLEKKLGYTFKDKSLLEKALTHVSYSKKEHYATLEFLGDALVNFFIVDLLVQYSPNKREGFLSPLKAYLISEEFFNLLAQKLELHKFIRIKRGKINETIIGDVFEALWAAVYIDSGRDANFTRELFYKLFKEDILSAIKEGRVKKDYKTILQEITQKRWKERPEYRLISVEGPHHKKKFIVEAKIKEYRTLGEGKSKKEAEQRAAEELIKLLEESE